SEQUENCE LISTING (90 sequences)

SEQ ID NO: 1 - protein, 5 aa, synthetic construct
  REGION 1..5: note = Synthetic_H-CDR1 of anti-4-1BB antibody
SYDMS

SEQ ID NO: 2 - protein, 5 aa, synthetic construct
  REGION 1..5: note = Synthetic_H-CDR1 of anti-4-1BB antibody
GYDMS

SEQ ID NO: 3 - protein, 5 aa, synthetic construct
  REGION 1..5: note = Synthetic_H-CDR1 of anti-4-1BB antibody
SYWMH

SEQ ID NO: 4 - protein, 17 aa, synthetic construct
  REGION 1..17: note = Synthetic_H-CDR2 of anti-4-1BB antibody
WISYSGGSIYYADSVKG

SEQ ID NO: 5 - protein, 17 aa, synthetic construct
  REGION 1..17: note = Synthetic_H-CDR2 of anti-4-1BB antibody
VIYPDDGNTYYADSVKG

SEQ ID NO: 6 - protein, 17 aa, synthetic construct
  REGION 1..17: note = Synthetic_H-CDR2 of anti-4-1BB antibody
EINPGNGHTNYNEKFKS

SEQ ID NO: 7 - protein, 12 aa, synthetic construct
  REGION 1..12: note = Synthetic_H-CDR3 of anti-4-1BB antibody
DGQRNSMREFDY

SEQ ID NO: 8 - protein, 12 aa, synthetic construct
  REGION 1..12: note = Synthetic_H-CDR3 of anti-4-1BB antibody
DAQRNSMREFDY

SEQ ID NO: 9 - protein, 12 aa, synthetic construct
  REGION 1..12: note = Synthetic_H-CDR3 of anti-4-1BB antibody
DAQRQSMREFDY

SEQ ID NO: 10 - protein, 18 aa, synthetic construct
  REGION 1..18: note = Synthetic_H-CDR3 of anti-4-1BB antibody
HGGQKPTTKSSSAYGMDG

SEQ ID NO: 11 - protein, 10 aa, synthetic construct
  REGION 1..10: note = Synthetic_H-CDR3 of anti-4-1BB antibody
SFTTARAFAY

SEQ ID NO: 12 - protein, 13 aa, synthetic construct
  REGION 1..13: note = Synthetic_L-CDR1 of anti-4-1BB antibody
SGSSSNIGNNYVT

SEQ ID NO: 13 - protein, 11 aa, synthetic construct
  REGION 1..11: note = Synthetic_L-CDR1 of anti-4-1BB antibody
RASQTISDYLH

SEQ ID NO: 14 - protein, 7 aa, synthetic construct
  REGION 1..7: note = Synthetic_L-CDR2 of anti-4-1BB antibody
ADSHRPS

SEQ ID NO: 15 - protein, 7 aa, synthetic construct
  REGION 1..7: note = Synthetic_L-CDR2 of anti-4-1BB antibody
YASQSIS

SEQ ID NO: 16 - protein, 11 aa, synthetic construct
  REGION 1..11: note = Synthetic_L-CDR3 of anti-4-1BB antibody
ATWDYSLSGYV

SEQ ID NO: 17 - protein, 9 aa, synthetic construct
  REGION 1..9: note = Synthetic_L-CDR3 of anti-4-1BB antibody
QDGHSFPPT

SEQ ID NO: 18 - protein, 121 aa, synthetic construct
  REGION 1..121: note = Synthetic_heavy chain variable region of anti-4-1BB antibody (1A10)
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDGQRNSMREFDYWGQGTLVTVSS

SEQ ID NO: 19 - protein, 121 aa, synthetic construct
  REGION 1..121: note = Synthetic_heavy chain variable region of anti-4-1BB antibody (1A10 M4, 1A10 M12)
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDAQRNSMREFDYWGQGTLVTVSS

SEQ ID NO: 20 - protein, 121 aa, synthetic construct
  REGION 1..121: note = Synthetic_heavy chain variable region of anti-4-1BB antibody (1A10 M11, 1A10 M13)
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDAQRQSMREFDYWGQGTLVTVSS

SEQ ID NO: 21 - protein, 127 aa, synthetic construct
  REGION 1..127: note = Synthetic_heavy chain variable region of anti-4-1BB antibody (1A12)
EVQLLESGGGLVQPGGSLRLSCAASGFTFSGYDMSWVRQAPGKGLEWVSVIYPDDGNTYYADSVKGRFTISRDNSKNTLYLQMNSLRAEDAAVYYCAKHGGQKPTTKSSSAYGMDGWGQGTLVTVSS

SEQ ID NO: 22 - protein, 127 aa, synthetic construct
  REGION 1..127: note = Synthetic_heavy chain variable region of anti-4-1BB antibody (1A12M1)
EVQLLESGGGLVQPGGSLRLSCAASGFTFSGYDMSWVRQAPGKGLEWVSVIYPDDGNTYYADSVKGRFTISRDNSKNTLYLQMNSLRAEDTAVYYCAKHGGQKPTTKSSSAYGMDGWGQGTLVTVSS

SEQ ID NO: 23 - protein, 119 aa, synthetic construct
  REGION 1..119: note = Synthetic_heavy chain variable region of anti-4-1BB antibody (AB41)
QVQLQQSGAEVIKPGASVKLSCKASGYTFSSYWMHWVRQAPGQGLEWIGEINPGNGHTNYNEKFKSRATLTGDTSTSTVYMELSSLRSEDTAVYYCARSFTTARAFAYWGQGTLVTVSS

SEQ ID NO: 24 - protein, 121 aa, synthetic construct
  REGION 1..121: note = Synthetic_heavy chain variable region of anti-4-1BB antibody (mutated 1A10)
EVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDGQRNSMREFDYWGQGTLVTVSS

SEQ ID NO: 25 - protein, 121 aa, synthetic construct
  REGION 1..121: note = Synthetic_heavy chain variable region of anti-4-1BB antibody (mutated 1A10 M4, 1A10 M12)
EVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 26 - protein, 121 aa, synthetic construct
  REGION 1..121: note = Synthetic_heavy chain variable region of anti-4-1BB antibody (mutated 1A10 M11, 1A10 M13)
EVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRQSMREFDYWGQGTLVTVSS

SEQ ID NO: 27 - protein, 127 aa, synthetic construct
  REGION 1..127: note = Synthetic_heavy chain variable region of anti-4-1BB antibody (mutated 1A12)
EVQLLESGGGLVQPGGSLRLSCAASGFTFSGYDMSWVRQAPGKCLEWVSVIYPDDGNTYYADSVKGRFTISRDNSKNTLYLQMNSLRAEDAAVYYCAKHGGQKPTTKSSSAYGMDGWGQGTLVTVSS

SEQ ID NO: 28 - protein, 127 aa, synthetic construct
  REGION 1..127: note = Synthetic_heavy chain variable region of anti-4-1BB antibody (mutated 1A12M1)
EVQLLESGGGLVQPGGSLRLSCAASGFTFSGYDMSWVRQAPGKCLEWVSVIYPDDGNTYYADSVKGRFTISRDNSKNTLYLQMNSLRAEDTAVYYCAKHGGQKPTTKSSSAYGMDGWGQGTLVTVSS

SEQ ID NO: 29 - protein, 119 aa, synthetic construct
  REGION 1..119: note = Synthetic_heavy chain variable region of anti-4-1BB antibody (mutated AB41)
QVQLQQSGAEVIKPGASVKLSCKASGYTFSSYWMHWVRQAPGQCLEWIGEINPGNGHTNYNEKFKSRATLTGDTSTSTVYMELSSLRSEDTAVYYCARSFTTARAFAYWGQGTLVTVSS

SEQ ID NO: 30 - protein, 110 aa, synthetic construct
  REGION 1..110: note = Synthetic_light chain variable region of anti-4-1BB antibody (1A10, 1A10 M4, 1A10 M11, 1A12)
QSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGGGTKLTVL

SEQ ID NO: 31 - protein, 110 aa, synthetic construct
  REGION 1..110: note = Synthetic_light chain variable region of anti-4-1BB antibody (1A10 M12, 1A10 M13, 1A12M1)
QSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGGGTKLTVL

SEQ ID NO: 32 - protein, 108 aa, synthetic construct
  REGION 1..108: note = Synthetic_light chain variable region of anti-4-1BB antibody (AB41)
DIVMTQSPAFLSVTPGEKVTITCRASQTISDYLHWYQQKPDQAPKLLIKYASQSISGIPSRFSGSGSGTDFTFTISSLEAEDAATYYCQDGHSFPPTFGQGTKLEIKR

SEQ ID NO: 33 - protein, 110 aa, synthetic construct
  REGION 1..110: note = Synthetic_light chain variable region of anti-4-1BB antibody (mutated 1A10, 1A10 M4, 1A10 M11, 1A12)
QSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGCGTKLTVL

SEQ ID NO: 34 - protein, 110 aa, synthetic construct
  REGION 1..110: note = Synthetic_light chain variable region of anti-4-1BB antibody (mutated 1A10 M12, 1A10 M13, 1A12M1)
QSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVL

SEQ ID NO: 35 - protein, 30 aa, synthetic construct
  REGION 1..30: note = Synthetic_H-FR1 of anti-4-1BB antibody
EVQLLESGGGLVQPGGSLRLSCAASGFTFS

SEQ ID NO: 36 - protein, 30 aa, synthetic construct
  REGION 1..30: note = Synthetic_H-FR1 of anti-4-1BB antibody
QVQLQQSGAEVIKPGASVKLSCKASGYTFS

SEQ ID NO: 37 - protein, 14 aa, synthetic construct
  REGION 1..14: note = Synthetic_H-FR2 of anti-4-1BB antibody
WVRQAPGKGLEWVS

SEQ ID NO: 38 - protein, 14 aa, synthetic construct
  REGION 1..14: note = Synthetic_H-FR2 of anti-4-1BB antibody
WVRQAPGQGLEWIG

SEQ ID NO: 39 - protein, 14 aa, synthetic construct
  REGION 1..14: note = Synthetic_H-FR2 of anti-4-1BB antibody
WVRQAPGKCLEWVS

SEQ ID NO: 40 - protein, 14 aa, synthetic construct
  REGION 1..14: note = Synthetic_H-FR2 of anti-4-1BB antibody
WVRQAPGQCLEWIG

SEQ ID NO: 41 - protein, 32 aa, synthetic construct
  REGION 1..32: note = Synthetic_H-FR3 of anti-4-1BB antibody
RFTISRDNSKNTLYLQMNSLRAEDTAVYYCAR

SEQ ID NO: 42 - protein, 32 aa, synthetic construct
  REGION 1..32: note = Synthetic_H-FR3 of anti-4-1BB antibody
RFTISRDNSKNTLYLQMNSLRAEDAAVYYCAK

SEQ ID NO: 43 - protein, 32 aa, synthetic construct
  REGION 1..32: note = Synthetic_H-FR3 of anti-4-1BB antibody
RFTISRDNSKNTLYLQMNSLRAEDTAVYYCAK

SEQ ID NO: 44 - protein, 32 aa, synthetic construct
  REGION 1..32: note = Synthetic_H-FR3 of anti-4-1BB antibody
RATLTGDTSTSTVYMELSSLRSEDTAVYYCAR

SEQ ID NO: 45 - protein, 11 aa, synthetic construct
  REGION 1..11: note = Synthetic_H-FR4 of anti-4-1BB antibody
WGQGTLVTVSS

SEQ ID NO: 46 - protein, 22 aa, synthetic construct
  REGION 1..22: note = Synthetic_L-FR1 of anti-4-1BB antibody
QSVLTQPPSASGTPGRRVTISC

SEQ ID NO: 47 - protein, 22 aa, synthetic construct
  REGION 1..22: note = Synthetic_L-FR1 of anti-4-1BB antibody
QSVLTQPPSASGTPGQRVTISC

SEQ ID NO: 48 - protein, 23 aa, synthetic construct
  REGION 1..23: note = Synthetic_L-FR1 of anti-4-1BB antibody
DIVMTQSPAFLSVTPGEKVTITC

SEQ ID NO: 49 - protein, 15 aa, synthetic construct
  REGION 1..15: note = Synthetic_L-FR2 of anti-4-1BB antibody
WYQQLPGTAPKLLIY

SEQ ID NO: 50 - protein, 15 aa, synthetic construct
  REGION 1..15: note = Synthetic_L-FR2 of anti-4-1BB antibody
WYQQKPDQAPKLLIK

SEQ ID NO: 51 - protein, 32 aa, synthetic construct
  REGION 1..32: note = Synthetic_L-FR3 of anti-4-1BB antibody
GVPDRFSGSKSGTSASLAISGLRSEDEADYYC

SEQ ID NO: 52 - protein, 32 aa, synthetic construct
  REGION 1..32: note = Synthetic_L-FR3 of anti-4-1BB antibody
GIPSRFSGSGSGTDFTFTISSLEAEDAATYYC

SEQ ID NO: 53 - protein, 10 aa, synthetic construct
  REGION 1..10: note = Synthetic_L-FR4 of anti-4-1BB antibody
FGGGTKLTVL

SEQ ID NO: 54 - protein, 11 aa, synthetic construct
  REGION 1..11: note = Synthetic_L-FR4 of anti-4-1BB antibody
FGQGTKLEIKR

SEQ ID NO: 55 - protein, 10 aa, synthetic construct
  REGION 1..10: note = Synthetic_L-FR4 of anti-4-1BB antibody
FGCGTKLTVL

SEQ ID NO: 56 - protein, 448 aa, synthetic construct
  REGION 1..448: note = Synthetic_heavy chain of anti-4-1BB antibody
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDGQRNSMREFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 448 aa, synthetic construct
  REGION 1..448: note = Synthetic_heavy chain of anti-4-1BB antibody
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDAQRNSMREFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 58 - protein, 448 aa, synthetic construct
  REGION 1..448: note = Synthetic_heavy chain of anti-4-1BB antibody
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDAQRQSMREFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 454 aa, synthetic construct
  REGION 1..454: note = Synthetic_heavy chain of anti-4-1BB antibody
EVQLLESGGGLVQPGGSLRLSCAASGFTFSGYDMSWVRQAPGKGLEWVSVIYPDDGNTYYADSVKGRFTISRDNSKNTLYLQMNSLRAEDAAVYYCAKHGGQKPTTKSSSAYGMDGWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 454 aa, synthetic construct
  REGION 1..454: note = Synthetic_heavy chain of anti-4-1BB antibody
EVQLLESGGGLVQPGGSLRLSCAASGFTFSGYDMSWVRQAPGKGLEWVSVIYPDDGNTYYADSVKGRFTISRDNSKNTLYLQMNSLRAEDTAVYYCAKHGGQKPTTKSSSAYGMDGWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - protein, 446 aa, synthetic construct
  REGION 1..446: note = Synthetic_heavy chain of anti-4-1BB antibody
QVQLQQSGAEVIKPGASVKLSCKASGYTFSSYWMHWVRQAPGQGLEWIGEINPGNGHTNYNEKFKSRATLTGDTSTSTVYMELSSLRSEDTAVYYCARSFTTARAFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - protein, 216 aa, synthetic construct
  REGION 1..216: note = Synthetic_light chain of anti-4-1BB antibody
QSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 63 - protein, 216 aa, synthetic construct
  REGION 1..216: note = Synthetic_light chain of anti-4-1BB antibody
QSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 64 - protein, 214 aa, synthetic construct
  REGION 1..214: note = Synthetic_light chain of anti-4-1BB antibody
DIVMTQSPAFLSVTPGEKVTITCRASQTISDYLHWYQQKPDQAPKLLIKYASQSISGIPSRFSGSGSGTDFTFTISSLEAEDAATYYCQDGHSFPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 5 aa, synthetic construct
  REGION 1..5: note = Synthetic_H-CDR1 of anti-HER2 antibody
DTYIH

SEQ ID NO: 66 - protein, 17 aa, synthetic construct
  REGION 1..17: note = Synthetic_H-CDR2 of anti-HER2 antibody
RIYPTNGYTRYADSVKG

SEQ ID NO: 67 - protein, 11 aa, synthetic construct
  REGION 1..11: note = Synthetic_H-CDR3 of anti-HER2 antibody
WGGDGFYAMDY

SEQ ID NO: 68 - protein, 11 aa, synthetic construct
  REGION 1..11: note = Synthetic_L-CDR1 of anti-HER2 antibody
RASQDVNTAVA

SEQ ID NO: 69 - protein, 7 aa, synthetic construct
  REGION 1..7: note = Synthetic_L-CDR2 of anti-HER2 antibody
SASFLYS

SEQ ID NO: 70 - protein, 9 aa, synthetic construct
  REGION 1..9: note = Synthetic_L-CDR3 of anti-HER2 antibody
QQHYTTPPT

SEQ ID NO: 71 - protein, 119 aa, synthetic construct
  REGION 1..119: note = Synthetic_heavy chain variable region of anti-HER2 antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVS

SEQ ID NO: 72 - protein, 108 aa, synthetic construct
  REGION 1..108: note = Synthetic_light chain variable region of anti-HER2 antibody
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKR

SEQ ID NO: 73 - protein, 450 aa, synthetic construct
  REGION 1..450: note = Synthetic_heavy chain of anti-HER2 antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 450 aa, synthetic construct
  REGION 1..450: note = Synthetic_heavy chain of anti-HER2 antibody (N297A)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 214 aa, synthetic construct
  REGION 1..214: note = Synthetic_light chain of anti-HER2 antibody
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 716 aa, synthetic construct
  REGION 1..716: note = Synthetic_heavy chain of bispecific antibody (HER2(NA)x1A10)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDGQRNSMREFDYWGQGTLVTVSS

SEQ ID NO: 77 - protein, 719 aa, synthetic construct
  REGION 1..719: note = Synthetic_heavy chain of bispecific antibody (HER2 (NA)x1A10 GSLH)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSGSGSGSGSGSGSQSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDGQRNSMREFDYWGQGTLVTVSS

SEQ ID NO: 78 - protein, 719 aa, synthetic construct
  REGION 1..719: note = Synthetic_heavy chain of bispecific antibody (HER2 (NA)x1A10 M4)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSGSGSGSGSGSGSQSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 79 - protein, 719 aa, synthetic construct
  REGION 1..719: note = Synthetic_heavy chain of bispecific antibody (HER2 (NA)x1A10 M12)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSGSGSGSGSGSGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 80 - protein, 722 aa, synthetic construct
  REGION 1..722: note = Synthetic_heavy chain of bispecific antibody (HER2 (NA)x1A12)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSGYDMSWVRQAPGKCLEWVSVIYPDDGNTYYADSVKGRFTISRDNSKNTLYLQMNSLRAEDAAVYYCAKHGGQKPTTKSSSAYGMDGWGQGTLVTVSS

SEQ ID NO: 81 - protein, 725 aa, synthetic construct
  REGION 1..725: note = Synthetic_heavy chain of bispecific antibody (HER2 (NA)x1A12 GSLH)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSGSGSGSGSGSGSQSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSGYDMSWVRQAPGKCLEWVSVIYPDDGNTYYADSVKGRFTISRDNSKNTLYLQMNSLRAEDAAVYYCAKHGGQKPTTKSSSAYGMDGWGQGTLVTVSS

SEQ ID NO: 82 - protein, 725 aa, synthetic construct
  REGION 1..725: note = Synthetic_heavy chain of bispecific antibody (HER2 (NA)x1A12 M1)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSGSGSGSGSGSGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSGYDMSWVRQAPGKCLEWVSVIYPDDGNTYYADSVKGRFTISRDNSKNTLYLQMNSLRAEDTAVYYCAKHGGQKPTTKSSSAYGMDGWGQGTLVTVSS

SEQ ID NO: 83 - protein, 719 aa, synthetic construct
  REGION 1..719: note = Synthetic_heavy chain of bispecific antibody (HER2 (WT)x1A10 M12)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSGSGSGSGSGSGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 84 - protein, 725 aa, synthetic construct
  REGION 1..725: note = Synthetic_heavy chain of bispecific antibody (HER2 (WT)x1A12)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSGSGSGSGSGSGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSGYDMSWVRQAPGKCLEWVSVIYPDDGNTYYADSVKGRFTISRDNSKNTLYLQMNSLRAEDTAVYYCAKHGGQKPTTKSSSAYGMDGWGQGTLVTVSS

SEQ ID NO: 85 - protein, 15 aa, synthetic construct
  REGION 1..15: note = Synthetic_peptide linker
GGGGSGGGGSGGGGS

SEQ ID NO: 86 - protein, 18 aa, synthetic construct
  REGION 1..18: note = Synthetic_peptide linker
GSGSGSGSGSGSGSGSGS

SEQ ID NO: 87 - protein, 20 aa, synthetic construct
  REGION 1..20: note = Synthetic_peptide linker
GGGGSGGGGSGGGGSGGGGS

SEQ ID NO: 88 - protein, 108 aa, synthetic construct
  REGION 1..108: note = Synthetic_light chain variable region of anti-4-1BB antibody (mutated AB41)
DIVMTQSPAFLSVTPGEKVTITCRASQTISDYLHWYQQKPDQAPKLLIKYASQSISGIPSRFSGSGSGTDFTFTISSLEAEDAATYYCQDGHSFPPTFGCGTKLEIKR

SEQ ID NO: 89 - protein, 255 aa, synthetic construct
  REGION 1..255: note = Synthetic_human 4-1BB (NP_001552.2)
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 90 - protein, 10 aa, synthetic construct
  REGION 1..10: note = Synthetic_peptide linker
GGGGSGGGGS

(19) United States
(12) Patent Application Publication
(10) Pub. No.: US 2025/0215112 A1
(43) Pub. Date: Jul. 3, 2025

(54) PHARMACEUTICAL COMPOSITION FOR TREATING OR PREVENTING CANCER WITH LOW EXPRESSION LEVEL OF HER2

(71) Applicant: YUHAN CORPORATION, Seoul (KR)

(72) Inventors: Eunjung LEE, Yongin-si (KR); Eun-Jung LEE, Yongin-si (KR); Jun Hwan KIM, Seoul (KR); Minji CHOI, Suwon-si (KR); Jang Woo SHIN, Yongin-si (KR); Hyejin CHUNG, Seongnam-si (KR); Yangsoon LEE, Seongnam-si (KR); Wonjun SON, Seongnam-si (KR)

(21) Appl. No.: 18/853,484

(22) PCT Filed: Apr. 6, 2023
(86) PCT No.: PCT/KR2023/004686

§ 371 Date: Oct. 2, 2024

(30) Foreign Application Priority Data
Apr. 7, 2022 (KR) 10-2022-0043555

(51) Int. Cl. C07K 16/32; A61P 35/00; C07K 16/28

(52) U.S. Cl. CPC C07K 16/32; A61P 35/00; C07K 16/2818; C07K 16/2878; C07K 2317/31; C07K 2317/622

(57) ABSTRACT

Provided are a pharmaceutical composition and a method for the treatment and/or prevention of HER2-low expressing and/or FcγRI-expressing cancers using anti-4-1BB/anti-HER2 bispecific antibodies.

### CROSS-REFERENCE TO RELATED APPLICATION

This application claims the benefit of priority from Korean Patent Application No. 10-2022-0043555, filed on Apr. 7, 2022, the entire disclosure of which is hereby incorporated by reference herein.

### TECHNICAL FIELD

Provided are a pharmaceutical composition and a method for the treatment and/or prevention of HER2 low-expressing and/or FcγRI-expressing cancers using anti-4-1BB/anti-HER2 bispecific antibodies.

### BACKGROUND ART

The 4-1BB protein is a member of the TNF-receptor superfamily (TNFRSF) and is a co-stimulatory molecule expressed after immune cell activation of both innate and adaptive immune cells. 4-1BB plays an important role in regulating the activity of various immune cells. 4-1BB agonists enhance immune cell proliferation and survival, cytokine secretion, and cytolytic-active CD8 T cells. Many other studies have shown that activation of 4-1BB enhances the immune response to eliminate tumors in mice, suggesting that 4-1BB is a promising target molecule in cancer immunology. Despite its antitumor efficacy, anti-4-1BB antibodies have caused severe liver toxicity in clinical applications.

On the other hand, the HER2 protein is a member of the epidermal growth factor receptor (EGFR) family and is involved in various mechanisms related to tumors. HER2 is a classical receptor tyrosine kinase (RTK) on the cell surface that induces cancer cell proliferation, invasion, and angiogenesis. Most of the HER2-targeted therapeutics developed to date show anti-cancer effects against cancers that express high levels of HER2, and do not show significant therapeutic effects against cancers that express low levels of HER2.

Accordingly, it is necessary to develop drugs with low liver toxicity and anti-cancer effects against a wide range of cancers that do not depend on HER2 expression levels for more efficient cancer treatment.

### DISCLOSURE

### Technical Problem

In one embodiment, provided is a pharmaceutical composition for the prevention and/or treatment of cancer comprising an anti-HER2/anti-4-1BB bispecific antibody comprising:
(1) an anti-HER2 antibody or antigen-binding fragment thereof as a HER2 targeting moiety capable of specifically recognizing and/or binding to the HER2 protein; and
(2) an anti-4-1BB antibody or antigen-binding fragment thereof as a 4-1BB targeting moiety capable of specifically recognizing and/or binding to 4-1BB protein.

The cancer may be a cancer characterized by low expression of HER2(HER2-low expression), expression of FcγRI, or both.

In another embodiment, provided is a method of preventing or treating cancer comprising the step of administering a pharmaceutically effective amount of said bispecific antibody or said pharmaceutical composition to a subject in need of prevention or treatment of cancer. The method may further comprise the step of identifying, prior to said step of administering, a subject in need of prevention or treatment of cancer. Said cancer may be a cancer characterized by HER2-low expression, FcγRI expression, or both.

In one embodiment, provide is said bispecific antibody or said pharmaceutical composition for use in the prevention or treatment of cancer.

Another embodiment provides a use of the bispecific antibody in preparing a pharmaceutical composition for the prevention or treatment of cancer. The cancer may be a cancer characterized by HER2-low expression, FcγRI expression, or both.

### Technical Solution

The present disclosure relates to bispecific antibodies comprising an antibody specific for tumor associated antigen (TAA; HER2) and an antibody specific for 4-1BB, and uses thereof. Said bispecific antibodies can activate 4-1BB signaling and enhance the immune response against HER2 high expression tumors (cancers), as well as cancer cells with HER2-low expression and/or FcγRI expression characteristics. Thus, the bispecific antibodies provided herein can be used as cancer immunotherapy agents that exhibit anticancer activity against tumors (cancers) with high HER2 expression as well as cancers characterized by HER2-low expression and/or FcγRI expression.

Provided herein are anti-cancer (including preventing, treating, ameliorating, alleviating, and/or curing) uses of an anti-HER2/anti-4-1BB bispecific antibody in a cancer having HER2-low expression and/or FcγRI expression characteristics, wherein the anti-HER2/anti-4-1BB bispecific antibody may comprise:
(1) an anti-HER2 antibody or antigen-binding fragment thereof as a HER2 targeting moiety capable of specifically recognizing and/or binding to HER2 protein; and
(2) an anti-4-1BB antibody or antigen-binding fragment thereof as a 4-1BB targeting moiety capable of specifically recognizing and/or binding to 4-1BB protein.

Hereinafter, the present invention will be described in more detail.

### DEFINITION OF TERMS

As used herein, “consisting of a sequence”, “consisting essentially of a sequence”, or “comprising a sequence” may refer to any case comprising the sequence, but is not intended to exclude a case comprising further sequence other than the sequence.

As used herein, the terms “a protein or polypeptide comprising or consisting of an amino acid sequence identified by SEQ ID NO” and “a gene or polynucleotide comprising or consisting of a nucleic acid sequence identified by SEQ ID NO” may refer to a protein (or a polypeptide) or a gene (or a polynucleotide), which consists essentially of the amino acid sequence or nucleic acid sequence, or which has at least 90%, at least 91%, at least 92%, at least 93%, at least 94%, at least 95%, at least 96%, at least 97%, at least 98%, or at least 99% sequence identity or similarity to said amino acid sequence or nucleic acid sequence, while retaining its inherent and/or intended activity and/or function.

As used herein, the term “antibody” can encompass a variety of broad classes of polypeptides that can be biochemically distinct. Those skilled in the art will understand that heavy chains are categorized as gamma, mu, alpha, delta, or epsilon (γ, μ, α, δ, ε), with some subclasses thereof (e.g., γ1-γ4), and light chains are categorized as kappa or lambda (K, λ). It is the nature of this chain that determines the “class” of antibody as IgG, IgM, IgA, IgD, or IgE, respectively. Immunoglobulin subclasses (isotypes), such as IgG1, IgG2, IgG3, IgG4, IgG5, etc. are well characterized and are known to confer functional specialization.

An intact antibody contains two full-length light chains and two full-length heavy chains, where each light chain can be linked to a heavy chain by a disulfide bond. The antibody has a heavy chain constant region and a light chain constant region. The heavy chain constant region is of the gamma (γ), mu (μ), alpha (α), delta (δ), or epsilon (ε) type, which may be further typified as gamma 1 (γ1), gamma 2 (γ2), gamma 3 (γ3), gamma 4 (γ4), alpha 1 (α1), or alpha 2 (α2). Light chain constant regions can be of the kappa (κ) or lambda (λ) type.

The term “heavy chain” may refer to a full-length heavy chain or a fragment thereof, including a variable region V_{H} that includes amino acid sequences sufficient to provide specificity to an antigen, and three constant regions, C_{H1}, C_{H2} and C_{H3}, and a hinge region. The term “light chain” may refer to a full-length light chain or a fragment thereof, including a variable region V_{L} that includes amino acid sequences sufficient to provide specificity to an antigen, and a constant region C_{L}.

The term “complementarity determining region (CDR)” refers to an amino acid sequence found in the hypervariable region of the heavy or light chain of an immunoglobulin. The heavy and light chains may respectively include three CDRs (CDRH1, CDRH2, and CDRH3; and CDRL1, CDRL2, and CDRL3). CDRs can provide residues that play a role in the binding of an antibody to an antigen or epitope. The terms “specifically binds” or “specifically recognized” are well known in the art and indicate that the antibody and antigen interact specifically to induce immunological activity.

In this disclosure, the antibody may include, but not be limited to, polyclonal or monoclonal; and/or human, humanized, animal (e.g., mouse, rabbit, etc.) derived antibody, or chimeric antibodies (e.g., mouse-human chimeric antibody).

Animal-derived antibodies, which are generated by immunizing an animal with a desired antigen, can cause immune rejection when administered to humans, usually for therapeutic purposes, and thus, chimeric antibodies have been developed to suppress such immune rejection. Chimeric antibodies are formed by replacing a constant region of an animal-derived antibody, which causes an anti-isotype response, with a constant region of a human antibody using genetic engineering methods. Although chimeric antibodies have significantly improved anti-isotypic responses compared to animal-derived antibodies, there are still potential side effects from anti-idiotypic responses because animal-derived amino acids are still present in their variable regions. Humanized antibodies have been developed to ameliorate these side effects. It is manufactured by transplanting the CDR (complementarity determining region), which plays an important role in antigen binding, among the variable regions of chimeric antibodies, into a human antibody framework.

As used herein, the term “antigen-binding fragment” refers to a fragment derived from a complete immunoglobulin structure that includes a portion capable of binding to an antigen, such as a CDR. For example, an antigen-binding fragment may be, but is not limited to, scFv, (scFv)2, scFv-Fc, Fab, Fab′, or F(ab′)2. For example, in the present disclosure, said antigen-binding fragment may be at least one antibody-derived fragment including CDR, selected from the group consisting of scFv, (scFv)2, scFv-Fc, Fab, Fab′, and F(ab′)2.

Among the antigen-binding fragments, Fab, which has a structure with variable regions of the light and heavy chains, a constant region of the light chain, and a first constant region (CH1) of the heavy chain, has one antigen-binding site.

Fab′ differs from Fab in that it has a hinge region containing one or more cysteine residues at the C-terminus of the heavy chain CH1 domain. F(ab′)2 antibodies are formed through disulfide bonds of cysteine residues in the hinge region of Fab′.

Fv is a minimal antibody fragment having only a heavy chain variable region and a light chain variable region, and recombinant techniques for producing Fv fragments are well known in the art. A two-chain Fv may have a structure in which the heavy chain variable region and light chain variable region are non-covalently linked, and a single-chain Fv (scFv) may generally have a dimer structure like a two-chain Fv in which the heavy chain variable region and light chain variable region are either covalently linked via a peptide linker, or directly linked to each other at the C-terminus thereof.

Antigen-binding fragments can be obtained using proteases (e.g., digestion of whole antibodies with papain to obtain Fab fragments or with pepsin to obtain F(ab′)2 fragments) and can be prepared by genetic recombination techniques.

An immunoglobulin (e.g., human immunoglobulin) or antibody molecule herein may be any type (e.g., IgG, IgE, IgM, IgD, IgA, IgY, etc.), class (e.g., IgG1, IgG2, IgG3, IgG4, IgG5, IgA1, IgA2, etc.), or subclass of immunoglobulin molecules.

In an antibody or antibody fragment, portions other than the CDR or variable region (e.g., constant regions) may be derived from a human antibody, and in particular, they may be derived from, for example, an IgG, IgA, IgD, IgE, IgM, or IgY, such as IgG1, IgG2, IgG3, or IgG4.

Antibodies or antigen-binding fragments can be synthesized chemically or recombinantly (not naturally occurring).

### 4-1BB Targeting Moiety

An anti-HER2/anti-4-1BB bispecific antibody may comprise an anti-4-1BB antibody or an antigen-binding fragment thereof as the 4-1BB targeting moiety.

The term “4-1BB”, also known as CD137 or TNF receptor superfamily member 9 (TNFRSF9), is a member of the TNF-receptor superfamily (TNFRSF) and is a co-stimulatory molecule expressed after immune cell activation of both innate and adaptive immune cells. 4-1BB plays an important role in regulating the activity of various immune cells. As used herein, 4-1BB can be derived from mammals, such as Homo sapiens (humans) (NCBI Accession No. NP_001552.2). For example, the human 4-1BB protein (NP_001552.2) can be represented by the amino acid sequence (SEQ ID NO: 89) as follows:
1 mgnscyniva tlllvlnfer trslqdpcsn cpagtfcdnn mqicspcpp nsfssaggqr
61 tcdicrqckg vfrtrkecss tsnaecdctp gfhclgagcs mceqdckqgq eltkkgckdc
121 cfgtfndqkr gicrpwtncs ldgksvlvng tkerdvvcgp spadlspgas svtppapare
181 pghspqiisf flaltstall fllffitlrf svvkrgrkkl lyifkqpfmr pvqttgeedg
241 cscrfpeeee ggcel

In one example, an anti-4-1BB antibody or an antigen-binding fragment thereof may comprise:
a complementarity determining region (CDR)-H1 (H-CDR1) comprising the amino acid sequence of SEQ ID NO: 1, 2, or 3;
an H-CDR2 comprising the amino acid sequence of SEQ ID NO: 4, 5, or 6; an H-CDR3 comprising the amino acid sequence of SEQ ID NO: 7, 8, 9, 10, or 11;
an L-CDR1 comprising the amino acid sequence of SEQ ID NO: 12 or 13;
an L-CDR2 comprising the amino acid sequence of SEQ ID NO: 14 or 15; and
an L-CDR3 comprising the amino acid sequence of SEQ ID NO: 16 or 17.

The amino acid sequence of the CDRs of an anti-4-1BB antibody or antigen-binding fragment are exemplified in Table 1:

TABLE 1

| SEQ ID | | SEQ ID | | SEQ ID | |
|---|---|---|---|---|---|
| NO | H-CDR1 | NO | H-CDR2 | NO | H-CDR3 |
| 1 | SYDMS | 4 | wisysggs | 7 | DGQRNSMREFDY |
| | | | iyyadsvk | 8 | DAQRNSMREFDY |
| | | | g | 9 | DAQRQSMREFDY |
| 2 | GYDMS | 5 | viypddgn | 10 | hggqkpttksss |
| | | | tyyadsvk | | saygmdg |
| | | | g | | |
| 3 | SYWMH | 6 | einpgngh | 11 | SFTTARAFAY |
| | | | tnynekfk | | |
| | | | s | | |
| SEQ ID | | SEQ ID | | SEQ ID | |
| NO | L-CDR1 | NO | L-CDR2 | NO | L-CDR3 |
| 12 | SGSSSN | 14 | ADSHRPS | 16 | ATWDYSLSGYV |
| | IGNNYV | | | | |
| | T | | | | |
| 13 | RASQTI | 15 | YASQSIS | 17 | QDGHSFPPT |
| | SDYLH | | | | |

For example, an anti-4-1BB antibody or an antigen-binding fragment thereof may comprise:
an H-CDR1 comprising the amino acid sequence of SEQ ID NO: 1, an H-CDR2 comprising the amino acid sequence of SEQ ID NO: 4, an H-CDR3 comprising the amino acid sequence of SEQ ID NO: 7, an L-CDR1 comprising the amino acid sequence of SEQ ID NO: 12, an L-CDR2 comprising the amino acid sequence of SEQ ID NO: 14, and an L-CDR3 comprising the amino acid sequence of SEQ ID NO: 16;
an H-CDR1 comprising the amino acid sequence of SEQ ID NO: 1, an H-CDR2 comprising the amino acid sequence of SEQ ID NO: 4, an H-CDR3 comprising the amino acid sequence of SEQ ID NO: 8, an L-CDR1 comprising the amino acid sequence of SEQ ID NO: 12, an L-CDR2 comprising the amino acid sequence of SEQ ID NO: 14, and an L-CDR3 comprising the amino acid sequence of SEQ ID NO: 16;
an H-CDR1 comprising the amino acid sequence of SEQ ID NO: 1, an H-CDR2 comprising the amino acid sequence of SEQ ID NO: 4, an H-CDR3 comprising the amino acid sequence of SEQ ID NO: 9, an L-CDR1 comprising the amino acid sequence of SEQ ID NO: 12, an L-CDR2 comprising the amino acid sequence of SEQ ID NO: 14, and an L-CDR3 comprising the amino acid sequence of SEQ ID NO: 16;
an H-CDR1 comprising the amino acid sequence of SEQ ID NO: 1, an H-CDR2 comprising the amino acid sequence of SEQ ID NO: 4, an H-CDR3 comprising the amino acid sequence of SEQ ID NO: 7, an L-CDR1 comprising the amino acid sequence of SEQ ID NO: 13, an L-CDR2 comprising the amino acid sequence of SEQ ID NO: 15, and an L-CDR3 comprising the amino acid sequence of SEQ ID NO: 17;
an H-CDR1 comprising the amino acid sequence of SEQ ID NO: 1, an H-CDR2 comprising the amino acid sequence of SEQ ID NO: 4, an H-CDR3 comprising the amino acid sequence of SEQ ID NO: 8, an L-CDR1 comprising the amino acid sequence of SEQ ID NO: 13, an L-CDR2 comprising the amino acid sequence of SEQ ID NO: 15, and an L-CDR3 comprising the amino acid sequence of SEQ ID NO: 17;
an H-CDR1 comprising the amino acid sequence of SEQ ID NO: 1, an H-CDR2 comprising the amino acid sequence of SEQ ID NO: 4, an H-CDR3 comprising the amino acid sequence of SEQ ID NO: 9, an L-CDR1 comprising the amino acid sequence of SEQ ID NO: 13, an L-CDR2 comprising the amino acid sequence of SEQ ID NO: 15, and an L-CDR3 comprising the amino acid sequence of SEQ ID NO: 17;
an H-CDR1 comprising the amino acid sequence of SEQ ID NO: 2, an H-CDR2 comprising the amino acid sequence of SEQ ID NO: 5, an H-CDR3 comprising the amino acid sequence of SEQ ID NO: 10, an L-CDR1 comprising the amino acid sequence of SEQ ID NO: 12, an L-CDR2 comprising the amino acid sequence of SEQ ID NO: 14, and an L-CDR3 comprising the amino acid sequence of SEQ ID NO: 16;
an H-CDR1 comprising the amino acid sequence of SEQ ID NO: 2, an H-CDR2 comprising the amino acid sequence of SEQ ID NO: 5, an H-CDR3 comprising the amino acid sequence of SEQ ID NO: 10, an L-CDR1 comprising the amino acid sequence of SEQ ID NO: 13, an L-CDR2 comprising the amino acid sequence of SEQ ID NO: 15, and an L-CDR3 comprising the amino acid sequence of SEQ ID NO: 17;
an H-CDR1 comprising the amino acid sequence of SEQ ID NO: 3, an H-CDR2 comprising the amino acid sequence of SEQ ID NO: 6, an H-CDR3 comprising the amino acid sequence of SEQ ID NO: 11, an L-CDR1 comprising the amino acid sequence of SEQ ID NO: 12, an L-CDR2 comprising the amino acid sequence of SEQ ID NO: 14, and an L-CDR3 comprising the amino acid sequence of SEQ ID NO: 16; or
an H-CDR1 comprising the amino acid sequence of SEQ ID NO: 3, an H-CDR2 comprising the amino acid sequence of SEQ ID NO: 6, an H-CDR3 comprising the amino acid sequence of SEQ ID NO: 11, an L-CDR1 comprising the amino acid sequence of SEQ ID NO: 13, an L-CDR2 comprising the amino acid sequence of SEQ ID NO: 15, and an L-CDR3 comprising the amino acid sequence of SEQ ID NO: 17.

In another example, an anti-4-1BB antibody or an antigen-binding fragment thereof may comprise:
a heavy chain variable region comprising an H-CDR1 comprising the amino acid sequence of SEQ ID NO: 1, 2, or 3, an H-CDR2 comprising the amino acid sequence of SEQ ID NO: 4, 5, or 6, and an H-CDR3 comprising the amino acid sequence of SEQ ID NO: 7, 8, 9, 10, or 11; and a light chain variable region comprising an L-CDR1 comprising the amino acid sequence of SEQ ID NO: 12 or 13, an L-CDR2 comprising the amino acid sequence of SEQ ID NO: 14 or 15, and an L-CDR3 comprising the amino acid sequence of SEQ ID NO: 16 or 17.

In another example, an anti-4-1BB antibody or an antigen-binding fragment thereof may comprise:
a heavy chain variable region comprising or consisting essentially of the amino acid sequence of SEQ ID NO: 18, 19, 20, 21, 22, 23, 24, 25, 26, 27, 28, or 29; and a light chain variable region comprising or consisting essentially of the amino acid sequence of SEQ ID NO: 30, 31, 32, 33, 34, or 88.

The amino acid sequences of the variable regions of anti-4-1BB antibodies or antigen-binding fragments are exemplified in Table 2:

TABLE 2

| SEQ ID | |
|---|---|
| NO | Heavy chain variable region targeting 4-1BB |
| 18 | EVQLLESGGGLVQPGGSLRLSCAASGFTFSSYDMSWVRQAPGKGLEWVSWISYS |
| | GGSIYYADSVKGRFTISRDNSKNTLYLQMNSLRAEDTAVYYCARDGQRNSMREFD |
| | YWGQGTLVTVSS |
| 19 | EVQLLESGGGLVQPGGSLRLSCAASGFTFSSYDMSWVRQAPGKGLEWVSWISYS |
| | GGSIYYADSVKGRFTISRDNSKNTLYLQMNSLRAEDTAVYYCARDAQRNSMREF |
| | DYWGQGTLVTVSS |
| 20 | EVQLLESGGGLVQPGGSLRLSCAASGFTFSSYDMSWVRQAPGKGLEWVSWISYS |
| | GGSIYYADSVKGRFTISRDNSKNTLYLQMNSLRAEDTAVYYCARDAQRQSMREF |
| | DYWGQGTLVTVSS |
| 21 | EVQLLESGGGLVQPGGSLRLSCAASGFTFSGYDMSWVRQAPGKGLEWVSVIYPD |
| | DGNTYYADSVKGRFTISRDNSKNTLYLQMNSLRAEDAAVYYCAKHGGQKPTTKSS |
| | SAYGMDGWGQGTLVTVSS |
| 22 | EVQLLESGGGLVQPGGSLRLSCAASGFTFSGYDMSWVRQAPGKGLEWVSVIYPD |
| | DGNTYYADSVKGRFTISRDNSKNTLYLQMNSLRAEDTAVYYCAKHGGQKPTTKSSS |
| | AYGMDGWGQGTLVTVSS |
| 23 | QVQLQQSGAEVIKPGASVKLSCKASGYTFSSYWMHWVRQAPGQGLEWIGEINPGN |
| | GHTNYNEKFKSRATLTGDTSTSTVYMELSSLRSEDTAVYYCARSFTTARAFAYWGQ |
| | GTLVTVSS |
| 24 | EVQLLESGGGLVQPGGSLRLSCAASGFTFSSYDMSWVRQAPGKCLEWVSWISYS |
| | GGSIYYADSVKGRFTISRDNSKNTLYLQMNSLRAEDTAVYYCARDGQRNSMREFD |
| | YWGQGTLVTVSS |
| 25 | EVQLLESGGGLVQPGGSLRLSCAASGFTFSSYDMSWVRQAPGKCLEWVSWISYS |
| | GGSIYYADSVKGRFTISRDNSKNTLYLQMNSLRAEDTAVYYCARDAQRNSMREFDY |
| | WGQGTLVTVSS |
| 26 | EVQLLESGGGLVQPGGSLRLSCAASGFTFSSYDMSWVRQAPGKCLEWVSWISYS |
| | GGSIYYADSVKGRFTISRDNSKNTLYLQMNSLRAEDTAVYYCARDAQRQSMREFDY |
| | WGQGTLVTVSS |
| 27 | EVQLLESGGGLVQPGGSLRLSCAASGFTFSGYDMSWVRQAPGKCLEWVSVIYPDD |
| | GNTYYADSVKGRFTISRDNSKNTLYLQMNSLRAEDAAVYYCAKHGGQKPTTKSSSA |
| | YGMDGWGQGTLVTVSS |
| 28 | EVQLLESGGGLVQPGGSLRLSCAASGFTFSGYDMSWVRQAPGKCLEWVSVIYPDD |
| | GNTYYADSVKGRFTISRDNSKNTLYLQMNSLRAEDTAVYYCAKHGGQKPTTKSSSA |
| | YGMDGWGQGTLVTVSS |
| 29 | QVQLQQSGAEVIKPGASVKLSCKASGYTFSSYWMHWVRQAPGQCLEWIGEINPGN |
| | GHTNYNEKFKSRATLTGDTSTSTVYMELSSLRSEDTAVYYCARSFTTARAFAYWGQ |
| | GTLVTVSS |
| SEQ ID | |
| NO | Light chain variable region targeting 4-1BB |
| 30 | QSVLTQPPSASGTPGRRVTISCSGSSSNIGNNYVTWYQQLPGTAPKLLIYADSHRP |
| | SGVPDRFSGSKSGTSASLAISGLRSEDEADYYCATWDYSLSGYVFGGGTKLTVL |
| 31 | QSVLTQPPSASGTPGQRVTISCSGSSSNIGNNYVTWYQQLPGTAPKLLIYADSHRP |
| | SGVPDRFSGSKSGTSASLAISGLRSEDEADYYCATWDYSLSGYVFGGGTKLTVL |
| 32 | DIVMTQSPAFLSVTPGEKVTITCRASQTISDYLHWYQQKPDQAPKLLIKYASQSISGI |
| | PSRFSGSGSGTDFTFTISSLEAEDAATYYCQDGHSFPPTFGQGTKLEIKR |
| 33 | QSVLTQPPSASGTPGRRVTISCSGSSSNIGNNYVTWYQQLPGTAPKLLIYADSHRP |
| | SGVPDRFSGSKSGTSASLAISGLRSEDEADYYCATWDYSLSGYVFGCGTKLTVL |
| 34 | QSVLTQPPSASGTPGQRVTISCSGSSSNIGNNYVTWYQQLPGTAPKLLIYADSHRP |
| | SGVPDRFSGSKSGTSASLAISGLRSEDEADYYCATWDYSLSGYVFGCGTKLTVL |
| 88 | DIVMTQSPAFLSVTPGEKVTITCRASQTISDYLHWYQQKPDQAPKLLIKYASQSISGI |
| | PSRFSGSGSGTDFTFTISSLEAEDAATYYCQDGHSFPPTFGCGTKLEIKR |

may comprise:
a heavy chain variable region comprising the amino acid sequence of SEQ ID NO: 18, 19, 20, 21, 22, 23, 24, 25, 26, 27, 28, or 29 and a light chain variable region comprising the amino acid sequence of SEQ ID NO: 30;
a heavy chain variable region comprising the amino acid sequence of SEQ ID NO: 18, 19, 20, 21, 22, 23, 24, 25, 26, 27, 28, or 29 and a light chain variable region comprising the amino acid sequence of SEQ ID NO: 31;
a heavy chain variable region comprising the amino acid sequence of SEQ ID NO: 18, 19, 20, 21, 22, 23, 24, 25, 26, 27, 28, or 29 and a light chain variable region comprising the amino acid sequence of SEQ ID NO: 32;
a heavy chain variable region comprising the amino acid sequence of SEQ ID NO: 18, 19, 20, 21, 22, 23, 24, 25, 26, 27, 28, or 29 and a light chain variable region comprising the amino acid sequence of SEQ ID NO: 33;
a heavy chain variable region comprising the amino acid sequence of SEQ ID NO: 18, 19, 20, 21, 22, 23, 24, 25, 26, 27, 28, or 29 and a light chain variable region comprising the amino acid sequence of SEQ ID NO: 34; or
a heavy chain variable region comprising the amino acid sequence of SEQ ID NO: 18, 19, 20, 21, 22, 23, 24, 25, 26, 27, 28, or 29 and a light chain variable region comprising the amino acid sequence of SEQ ID NO: 88.

The amino acid sequences of the frameworks of the variable regions of the anti-4-1BB antibody or antigen-binding fragment are exemplified in Table 3:

TABLE 3

| SEQ | | SEQ | | SEQ | | SEQ | |
|---|---|---|---|---|---|---|---|
| ID | | ID | | ID | | ID | |
| NO | H-FR1 | NO | H-FR2 | NO | H-FR3 | NO | H-FR4 |
| 35 | EVQLLESGGGL | 37 | WVRQAPGK | 41 | RFTISRDNSKN | 45 | WGQGTL |
| | VQPGGSLRLS | | GLEWVS | | TLYLQMNSLRA | | VTVSS |
| | CAASGFTFS | | | | EDTAVYYCAR | | |
| 35 | EVQLLESGGGL | 37 | WVRQAPGK | 42 | RFTISRDNSKN | 45 | WGQGTL |
| | VQPGGSLRLS | | GLEWVS | | TLYLQMNSLRA | | VTVSS |
| | CAASGFTFS | | | | EDAAVYYCAK | | |
| 35 | EVQLLESGGGL | 37 | WVRQAPGK | 43 | RFTISRDNSKN | 45 | WGQGTL |
| | VQPGGSLRLS | | GLEWVS | | TLYLQMNSLRA | | VTVSS |
| | CAASGFTFS | | | | EDTAVYYCAK | | |
| 36 | QVQLQQSGAE | 38 | WVRQAPGQ | 44 | RATLTGDTSTS | 45 | WGQGTL |
| | VIKPGASVKLS | | GLEWIG | | TVYMELSSLRS | | VTVSS |
| | CKASGYTFS | | | | EDTAVYYCAR | | |
| 35 | EVQLLESGGGL | 39 | WVRQAPGK | 41 | RFTISRDNSKN | 45 | WGQGTL |
| | VQPGGSLRLS | | CLEWVS | | TLYLQMNSLRA | | VTVSS |
| | CAASGFTFS | | | | EDTAVYYCAR | | |
| 35 | EVQLLESGGGL | 39 | WVRQAPGK | 42 | RFTISRDNSKN | 45 | WGQGTL |
| | VQPGGSLRLS | | CLEWVS | | TLYLQMNSLRA | | VTVSS |
| | CAASGFTFS | | | | EDAAVYYCAK | | |
| 35 | EVQLLESGGGL | 39 | WVRQAPGK | 43 | RFTISRDNSKN | 45 | WGQGTL |
| | VQPGGSLRLS | | CLEWVS | | TLYLQMNSLRA | | VTVSS |
| | CAASGFTFS | | | | EDTAVYYCAK | | |
| 36 | QVQLQQSGAE | 40 | WVRQAPGQ | 44 | RATLTGDTSTS | 45 | WGQGTL |
| | VIKPGASVKLS | | CLEWIG | | TVYMELSSLRS | | VTVSS |
| | CKASGYTFS | | | | EDTAVYYCAR | | |
| SEQ | | SEQ | | SEQ | | SEQ | |
| ID | | ID | | ID | | ID | |
| NO | L-FR1 | NO | L-FR2 | NO | L-FR3 | NO | LFR4 |
| 46 | QSVLTQPPSAS | 49 | WYQQLPGT | 51 | GVPDRFSGSK | 53 | FGGGTK |
| | GTPGRRVTISC | | APKLLIY | | SGTSASLAISG | | LTVL |
| | | | | | LRSEDEADYYC | | |
| 47 | QSVLTQPPSAS | 49 | WYQQLPGT | 51 | GVPDRFSGSK | 53 | FGGGTK |
| | GTPGQRVTISC | | APKLLIY | | SGTSASLAISG | | LTVL |
| | | | | | LRSEDEADYYC | | |
| 48 | DIVMTQSPAFL | 50 | WYQQKPDQ | 52 | GIPSRFSGSGS | 54 | FGQGTK |
| | SVTPGEKVTIT | | APKLLIK | | GTDFTFTISSLE | | LEIKR |
| | C | | | | AEDAATYYC | | |
| 46 | QSVLTQPPSAS | 49 | WYQQLPGT | 51 | GVPDRFSGSK | 55 | FGCGTK |
| | GTPGRRVTISC | | APKLLIY | | SGTSASLAISG | | LTVL |
| | | | | | LRSEDEADYYC | | |
| 47 | QSVLTQPPSAS | 49 | WYQQLPGT | 51 | GVPDRFSGSK | 55 | FGCGTK |
| | GTPGQRVTISC | | APKLLIY | | SGTSASLAISG | | LTVL |
| | | | | | LRSEDEADYYC | | |

In another example, the anti-4-1BB antibody or antigen-binding fragment thereof may comprise a heavy chain comprising or consisting essentially of the amino acid sequence of SEQ ID NO: 56, 57, 58, 59, 60, or 61; and a light chain comprising or consisting essentially of the amino acid sequence of SEQ ID NO: 62, 63, or 64.

For example, the anti-4-1BB antibody or antigen-binding fragment thereof may comprise:
a heavy chain comprising or consisting essentially of the amino acid sequence of SEQ ID NO: 56, 57, 58, 59, 60, or 61; and a light chain comprising or consisting essentially of the amino acid sequence of SEQ ID NO: 62;
a heavy chain comprising or consisting essentially of the amino acid sequence of SEQ ID NO: 56, 57, 58, 59, 60, or 61; and a light chain comprising or consisting essentially of the amino acid sequence of SEQ ID NO: 63; or
a heavy chain comprising or consisting essentially of an amino acid sequence of SEQ ID NO: 56, 57, 58, 59, 60, or 61; and a light chain comprising or consisting essentially of the amino acid sequence of SEQ ID NO: 64.

In another example, the anti-4-1BB antibody or antigen-binding fragment thereof may be a scFv (single chain variable fragment) comprising: a heavy chain variable region comprising an H-CDR1 comprising the amino acid sequence of SEQ ID NO: 1, 2, or 3, an H-CDR2 comprising the amino acid sequence of SEQ ID NO: 4, 5, or 6, and an H-CDR3 comprising the amino acid sequence of SEQ ID NO: 7, 8, 9, 10, or 11; and
a light chain variable region comprising an L-CDR1 comprising the amino acid sequence of SEQ ID NO: 12 or 13, an L-CDR2 comprising the amino acid sequence of SEQ ID NO: 14 or 15, and an L-CDR3 comprising the amino acid sequence of SEQ ID NO: 16 or 17,
wherein the heavy chain variable region and the light chain variable region may be linked to each other in any order, either directly (e.g., without linkers) or via a peptide linker.

In one example, the scFv of the anti-4-1BB may comprise:
a heavy chain variable region comprising or consisting essentially of the amino acid sequence of SEQ ID NO: 18, 19, 20, 21, 22, 23, 24, 25, 26, 27, 28, or 29; and
a light chain variable region comprising or consisting essentially of the amino acid sequence of SEQ ID NO: 30, 31, 32, 33, 34, or 88,
wherein the heavy chain variable region and the light chain variable region may be linked to each other in any order, either directly or via a peptide linker.

In one example, the scFv of the anti-4-1BB may comprise:
a heavy chain variable region comprising or consisting essentially of the amino acid sequence of SEQ ID NO: 24, 25, 26, 27, 28, or 29 and a light chain variable region comprising or consisting essentially of the amino acid sequence of SEQ ID NO: 33;
a heavy chain variable region comprising or consisting essentially of the amino acid sequence of SEQ ID NO: 24, 25, 26, 27, 28, or 29 and a light chain variable region comprising or consisting essentially of the amino acid sequence of SEQ ID NO: 34; or
a heavy chain variable region comprising or consisting essentially of the amino acid sequence of SEQ ID NO: 24, 25, 26, 27, 28, or 29 and a light chain variable region comprising or consisting essentially of the amino acid sequence of SEQ ID NO: 88,
wherein the heavy chain variable region and the light chain variable region may be linked to each other in any order, either directly or via a peptide linker.

In the present disclosure, the anti-4-1BB scFv comprises a heavy chain variable region and a light chain variable region in any order. For example, an anti-4-1BB scFv may comprise a light chain variable region and a heavy chain variable region in an N-terminal to C-terminal direction. Alternatively, the anti-4-1BB scFv can comprise a heavy chain variable region and a light chain variable region in the N-terminal to C-terminal direction. In one example, an anti-4-1BB scFv may comprise a light chain variable region, a peptide linker, and a heavy chain variable region in an N-terminal to C-terminal direction. In another example, the anti-4-1BB scFv may comprise a heavy chain variable region, a peptide linker, and a light chain variable region in an N-terminal to C-terminal direction.

### HER2 Targeting Moiety

An anti-HER2/anti-4-1BB bispecific antibody may comprise an anti-HER2 antibody or an antigen-binding fragment thereof as the HER2 targeting moiety.

“HER2 (human epidermal growth factor receptor 2)” is encoded by the ERBB2 gene and is a member of the epidermal growth factor receptor (EGFR/ErbB) family. HER2 is known to play an essential role in regulating cell proliferation and differentiation. In particular, upon binding to extracellular growth factors, it has a strong tendency to assemble into homodimers and/or heterodimers with other HER receptors, activating several forms of signal transduction pathways to induce cell death, survival, or cell proliferation. For example, the HER2 protein may be a polypeptide deposited under GenBank Accession Nos. NP_004439.2, NP_001005862.1, etc., which is encoded by a nucleotide sequence (mRNA) deposited under GenBank Accession Nos. NM_004448.4, NM_001005862.3, etc., respectively.

In one embodiment, the anti-HER2 antibody may be selected from the group consisting of trastuzumab, pertuzumab, and trastuzumab emtansine (T-DM1).

The antigen-binding region of an anti-HER2 antibody that recognizes HER2 as an antigen can be scFv, (scFv)2, Fab, Fab′, or F(ab′)2 of an anti-HER2 antibody selected from the group consisting of trastuzumab, pertuzumab, and trastuzumab emtansine.

The anti-HER2 antibody or antigen-binding fragment thereof may be an anti-HER2 antibody or antigen-binding fragment thereof comprising the six CDRs of trastuzumab, pertuzumab, or trastuzumab emtansine.

In another embodiment, the anti-HER2 antibody or antigen-binding fragment thereof may be trastuzumab or an antigen-binding fragment thereof, or a variant thereof.

For example, said anti-HER2 antibody or antigen-binding fragment thereof may comprise:
an H-CDR1 comprising the amino acid sequence of SEQ ID NO: 65;
an H-CDR2 comprising the amino acid sequence of SEQ ID NO: 66;
an H-CDR3 comprising the amino acid sequence of SEQ ID NO: 67;
an L-CDR1 comprising the amino acid sequence of SEQ ID NO: 68;
an L-CDR2 comprising the amino acid sequence of SEQ ID NO: 69; and
an L-CDR3 comprising the amino acid sequence of SEQ ID NO: 70.

The amino acid sequences of the CDRs of anti-HER2 antibodies or antigen-binding fragments are exemplified in Table 4:

TABLE 4

| SEQ | VH_ | SEQ | VH_ | SEQ | VH_ |
|---|---|---|---|---|---|
| ID NO | CDR1 | ID NO | CDR2 | ID NO | CDR3 |
| 65 | DTYIH | 66 | RIYPTNG | 67 | WGGDGF |
| | | | YTRYADS | | YAMDY |
| | | | VKG | | |
| SEQ | | SEQ | | SEQ | |
| ID NO | L-CDR1 | ID NO | L-CDR2 | ID NO | L-CDR3 |
| 68 | RASQDV | 69 | SASFLY | 70 | QQHYTT |
| | NTAVA | | S | | PPT |

In another example, the anti-HER2 antibody or antigen-binding fragment thereof may comprise:
a heavy chain variable region comprising an H-CDR1 comprising the amino acid sequence of SEQ ID NO: 65, an H-CDR2 comprising the amino acid sequence of SEQ ID NO: 66, and an H-CDR3 comprising the amino acid sequence of SEQ ID NO: 67; and a light chain variable region comprising an L-CDR1 comprising the amino acid sequence of SEQ ID NO: 68, an L-CDR2 comprising the amino acid sequence of SEQ ID NO: 69, and an L-CDR3 comprising the amino acid sequence of SEQ ID NO: 70.

In another example, the anti-HER2 antibody or antigen-binding fragment thereof may comprise:
a heavy chain variable region comprising or consisting essentially of the amino acid sequence of SEQ ID NO: 71; and a light chain variable region comprising or consisting essentially of the amino acid sequence of SEQ ID NO: 72.

The amino acid sequences of the variable regions of anti-HER2 antibodies or antigen-binding fragments are exemplified in Table 5:

TABLE 5

| SEQ ID | Heavy Chain Variable |
|---|---|
| NO | Region Targeting HER2 |
| 71 | EVQLVESGGGLVQPGGSLRLSCAASGFNI |
| | KDTYIHWVRQAPGKGLEWVARIYPTNGYT |
| | RYADSVKGRFTISADTSKNTAYLQMNSLR |
| | AEDTAVYYCSRWGGDGFYAMDYWGQGTLV |
| | TVS |
| SEQ ID | Light Chain Variable |
| NO | Region Targeting HER2 |
| 72 | DIQMTQSPSSLSASVGDRVTITCRASQDV |
| | NTAVAWYQQKPGKAPKLLIYSASFLYSGV |
| | PSRFSGSRSGTDFTLTISSLQPEDFATYY |
| | CQQHYTTPPTFGQGTKVEIKR |

In another embodiment, the anti-HER2 antibody or antigen-binding fragment thereof may comprise:
a heavy chain comprising or consisting essentially of the amino acid sequence of SEQ ID NO: 73 or 74; and a light chain comprising or consisting essentially of the amino acid sequence of SEQ ID NO: 75.

In another embodiment, the anti-HER2 antibody or antigen-binding fragment thereof may be a scFv (single chain variable fragment) comprising:
a heavy chain variable region comprising an H-CDR1 comprising the amino acid sequence of SEQ ID NO: 65, an H-CDR2 comprising the amino acid sequence of SEQ ID NO: 66, and an H-CDR3 comprising the amino acid sequence of SEQ ID NO: 67; and
a light chain variable region comprising an L-CDR1 comprising the amino acid sequence of SEQ ID NO: 68, an L-CDR2 comprising the amino acid sequence of SEQ ID NO: 69, and an L-CDR3 comprising the amino acid sequence of SEQ ID NO: 70,
wherein the heavy chain variable regions and the light chain variable regions may be linked to each other in any order, either directly (e.g., without linkers) or via a peptide linker.

In another embodiment, the anti-HER2 antibody or antigen-binding fragment thereof may be a scFv (single chain variable fragment) comprising:
a heavy chain variable region comprising or consisting essentially of the amino acid sequence of SEQ ID NO: 71; and a light chain variable region comprising or consisting essentially of the amino acid sequence of SEQ ID NO: 72,
wherein the heavy chain variable region and the light chain variable region may be linked to each other in any order, either directly or via a peptide linker.

In the present disclosure, an anti-HER2 scFv may comprise a heavy chain variable region and a light chain variable region in any order. For example, an anti-HER2 scFv may comprise a light chain variable region and a heavy chain variable region in an N-terminal to C-terminal direction. Alternatively, an anti-HER2 scFv may comprise a heavy chain variable region and a light chain variable region in the N-terminal to C-terminal direction. In one example, an anti-HER2 scFv may comprise a light chain variable region, a peptide linker, and a heavy chain variable region in an N-terminal to C-terminal direction. In another example, an anti-HER2 scFv may comprise a heavy chain variable region, a peptide linker, and a light chain variable region in an N-terminal to C-terminal direction.

### Bispecific Antibodies

The present disclosure provides anti-HER2/anti-4-1BB bispecific antibodies comprising:
(1) an anti-HER2 antibody or antigen-binding fragment thereof as a HER2 targeting moiety capable of specifically recognizing and/or binding to HER2 protein; and
(2) an anti-4-1BB antibody or antigen-binding fragment thereof as a 4-1BB targeting moiety capable of specifically recognizing and/or binding to 4-1BB protein.

The anti-HER2/anti-4-1BB bispecific antibodies may activate 4-1BB signaling only when cross-linked by HER2-expressing tumor cells. Further, the anti-4-1BB antibody or antigen-binding fragment thereof included in the bispecific antibody may be characterized by localization and/or activation only in the tumor microenvironment (TME), and/or significantly reduced liver toxicity compared to conventional anti-4-1BB antibodies, while maintaining immune response enhancement and/or efficacy of tumor therapy.

In one embodiment, the bispecific antibody may comprise a full-length anti-HER2 antibody and an antigen-binding fragment (e.g., scFv) of an anti-4-1BB antibody, wherein the antigen-binding fragment of anti-4-1BB can be linked to the N-terminus, C-terminus, or both of the full-length anti-HER2 antibody directly or via a peptide linker. In another embodiment, the bispecific antibody may comprise a full-length anti-4-1BB antibody and an antigen-binding fragment (e.g., scFv) of an anti-HER2 antibody, wherein the antigen-binding fragment of the anti-HER2 antibody may be linked to the N-terminus, C-terminus, or both of the full-length anti-4-1BB antibody directly or via a peptide linker.

In one embodiment, the scFv contained in the bispecific antibody may comprise a heavy chain variable region and a light chain variable region in any order. For example, the scFv contained in the bispecific antibody may comprise a light chain variable region and a heavy chain variable region in an N-terminal to C-terminal direction, and optionally include a peptide linker between them, or the scFv contained in the bispecific antibody may comprise a heavy chain variable region and a light chain variable region in an N-terminal to C-terminal direction, and optionally include a peptide linker between them.

When the bispecific antibody comprises a full-length anti-HER2 antibody and an anti-4-1BB scFv, the bispecific antibody may comprise:
(i) a first polypeptide comprising, in an N-terminal to C-terminal direction:
a heavy chain of an anti-HER2 antibody,
optionally, a peptide linker (a first peptide linker), and
an anti-4-1BB scFv; and
(ii) a second polypeptide comprising a light chain of an anti-HER2 antibody,
wherein the anti-4-1BB scFv may comprise, in an N-terminal to C-terminal direction:
a light chain variable region of an anti-4-1BB antibody,
optionally, a peptide linker (a second peptide linker), and
a heavy chain variable region of an anti-4-1BB antibody.

Alternatively, the bispecific antibody may comprise:
(i) a first polypeptide comprising, in an N-terminal to C-terminal direction:
an anti-4-1BB scFv;
optionally, a peptide linker (a first peptide linker), and
a heavy chain of an anti-HER2 antibody; and
(ii) a second polypeptide comprising a light chain of an anti-HER2 antibody,
wherein the anti-4-1BB scFv may comprise, in an N-terminal to C-terminal direction:
a light chain variable region of an anti-4-1BB antibody,
optionally, a peptide linker (a second peptide linker), and
a heavy chain variable region of an anti-4-1BB antibody.

Alternatively, the bispecific antibody may comprise:
(i) a first polypeptide comprising, in an N-terminal to C-terminal direction:
a heavy chain of an anti-HER2 antibody;
optionally, a peptide linker (a first peptide linker), and
an anti-4-1BB scFv; and
(ii) a second polypeptide comprising a light chain of an anti-HER2 antibody,
wherein the anti-4-1BB scFv may comprise, in an N-terminal to C-terminal direction:
a heavy chain variable region of an anti-4-1BB antibody,
optionally, a peptide linker (a second peptide linker), and
a light chain variable region of an anti-4-1BB antibody.

Alternatively, the bispecific antibody may comprise:
(i) a first polypeptide comprising, in an N-terminal to C-terminal direction:
an anti-4-1BB scFv;
optionally, a peptide linker (a first peptide linker), and
a heavy chain of an anti-HER2 antibody; and
(ii) a second polypeptide comprising a light chain of an anti-HER2 antibody,
wherein the anti-4-1BB scFv may comprise, in an N-terminal to C-terminal direction:
a heavy chain variable region of an anti-4-1BB antibody, optionally, a peptide linker (a second peptide linker), and
a light chain variable region of an anti-4-1BB antibody.

When the bispecific antibody comprises a full-length anti-4-1BB antibody and an anti-HER2 scFv, the bispecific antibody may comprise:
(i) a first polypeptide comprising, in an N-terminal to C-terminal direction:
a heavy chain of an anti-4-1BB antibody,
optionally, a peptide linker (a first peptide linker), and
an anti-HER2 scFv; and
(ii) a second polypeptide comprising a light chain of an anti-4-1BB antibody,
wherein the anti-HER2 scFv may comprise, in an N-terminal to C-terminal direction:
a light chain variable region of an anti-HER2 antibody,
optionally, a peptide linker (a second peptide linker), and
a heavy chain variable region of an anti-HER2 antibody.

Alternatively, the bispecific antibody may comprise:
(i) a first polypeptide comprising, in an N-terminal to C-terminal direction:
an anti-HER2 scFv;
optionally, a peptide linker (a first peptide linker), and
a heavy chain of an anti-4-1BB antibody; and
(ii) a second polypeptide comprising a light chain of an anti-4-1BB antibody,
wherein the anti-HER2 scFv may comprise, in an N-terminal to C-terminal direction:
a light chain variable region of an anti-HER2 antibody, optionally, a peptide linker (a second peptide linker), and
a heavy chain variable region of an anti-HER2 antibody.

Alternatively, the bispecific antibody may comprise:
(i) a first polypeptide comprising, in an N-terminal to C-terminal direction:
a heavy chain of an anti-4-1BB antibody,
optionally, a peptide linker (a first peptide linker), and
an anti-HER2 scFv; and
(ii) a second polypeptide comprising a light chain of an anti-4-1BB antibody,
wherein the anti-HER2 scFv may comprise, in an N-terminal to C-terminal direction:
a heavy chain variable region of an anti-HER2 antibody,
optionally, a peptide linker (a second peptide linker), and
a light chain variable region of an anti-HER2 antibody.

Alternatively, the bispecific antibody may comprise:
(i) a first polypeptide comprising, in an N-terminal to C-terminal direction:
an anti-HER2 scFv,
optionally, a peptide linker (a first peptide linker), and
a heavy chain of an anti-4-1BB antibody; and
(ii) a second polypeptide comprising a light chain of an anti-4-1BB antibody,
wherein the anti-HER2 scFv may comprise, in an N-terminal to C-terminal direction:
a heavy chain variable region of an anti-HER2 antibody,
optionally, a peptide linker (a second peptide linker), and
a light chain variable region of an anti-HER2 antibody.

The first peptide linker and the second peptide linker may or may not be independently present in the bispecific antibody, and may be the same or different from each other.

In one embodiment, the anti-HER2/anti-4-1BB bispecific antibody may comprise:
(i) a first polypeptide comprising the amino acid sequence of SEQ ID NO: 76, 77, 78, 79, 80, 81, 82, 83, or 84; and
(ii) a second polypeptide comprising the amino acid sequence of SEQ ID NO: 75.

For example, the anti-HER2/anti-4-1BB bispecific antibody may include:
(i) a first polypeptide comprising the amino acid sequence of SEQ ID NO: 83; and
(ii) a second polypeptide comprising the amino acid sequence of SEQ ID NO: 75.

In another embodiment, both the HER2 targeting moiety and the 4-1BB targeting moiety included in the bispecific antibody may be a full-length antibody or an antigen-binding fragment comprising heavy chain CDRs, light chain CDRs, or a combination thereof, and may be linked to each other via a peptide linker or directly.

Given that each antibody can bind to both 4-1BB (e.g., human 4-1BB) and HER2 (e.g., human HER2), CDR sequences, or VH (heavy chain variable region) and VL (light chain variable region) sequences as disclosed herein can be “mixed and matched” to create different anti-HER2/anti-4-1BB bispecific molecules.

### Peptide Linkers

For high purity of the antibody, the bispecific antibody may comprise a peptide linker between the heavy chain of the first polypeptide and scFv (a first peptide linker) and/or between the heavy chain and light chain variable regions of scFv (a second peptide linker).

As used herein, the term “peptide linker” may refer to an oligopeptide comprising from 1 to 100 amino acids, in particular from 2 to 50 amino acids, each of which may be any kind of amino acid without limitation. Any conventional peptide linker may be used, with or without appropriate modifications, to accomplish a particular purpose. In certain embodiments, the peptide linker may comprise, for example, Gly, Asn, and/or Ser residues, and/or may comprise a neutral amino acid such as Thr and/or Ala. Suitable amino acid sequences for peptide linkers may be known in the related art. The length of the peptide linker may be suitably determined to the extent that the function of the polypeptide and/or scFv is not affected. For example, the peptide linker may be about 1 to about 100 amino acids, about 2 to about 50 amino acids, or about 5 to about 25 amino acids (e.g., 1, 2, 3, 4, 5, 6, 7, 8, 9, 10, 11, 12, 13, 14, 15, 16, 17, 18, 19, 20, 21, 22, 23, 24, or 25) amino acids, each of which may be independently selected from the group consisting of Gly, Asn, Ser, Thr, and Ala. In one embodiment, the peptide linker may be expressed as (Gₘ Sₗ)ₙ (where m, l, and n are the number of “G”, “S”, and “(Gₘ Sₗ)”, respectively, and each may be independently selected from an integer from about 1 to about 10, particularly 1, 2, 3, 4, 5, 6, 7, 8, 9, or 10). In one embodiment, the peptide linker may be a peptide of (GGGGS)2 (SEQ ID NO: 90), (GGGGS)3 (SEQ ID NO: 85), (GGGGS)4 (SEQ ID NO: 87), or (GS)9 (SEQ ID NO: 87), but is not limited thereto.

### Medicinal Uses

A medicinal use of the bispecific antibody for the prevention and/or treatment of cancer is provided.

More specifically, the present disclosure provides a pharmaceutical composition comprising the bispecific antibody as an active ingredient. The pharmaceutical composition may further comprise a pharmaceutically acceptable carrier. The pharmaceutical composition may further comprise a cancer immunotherapy agent. The bispecific antibody and the cancer immunotherapy agent may be included together in the form of a fusion protein or a multi-specific antibody (e.g., a trispecific antibody or a tetraspecific antibody, etc.), or formulated for co-administration. In one embodiment, the bispecific antibody and the cancer immunotherapy agent may be formulated in a single preparation, or the bispecific antibody and the cancer immunotherapy agent may be formulated separately and then mixed, but are not limited thereto. The pharmaceutical composition may be used for the prevention and/or treatment of cancer.

Another embodiment provides a pharmaceutical composition for the prevention and/or treatment of cancer comprising the bispecific antibody as an active ingredient. The pharmaceutical composition may further comprise a cancer immunotherapy agent. The bispecific antibody and cancer immunotherapy agent may be included together in the form of a fusion protein or a multi-specific antibody (e.g., a trispecific antibody or a tetraspecific antibody, etc.), or formulated for co-administration. In one embodiment, the bispecific antibody and the cancer immunotherapy agent may be formulated in a single preparation, or the bispecific antibody and the cancer immunotherapy agent may be formulated separately and then mixed, but are not limited thereto.

Another embodiment provides a method of preventing and/or treating cancer, comprising administering a pharmaceutically effective amount of the bispecific antibody or the pharmaceutical composition to a subject in need thereof. The method may further comprise identifying a subject in need of prevention and/or treatment of cancer prior to the step of administering. The method may further comprise administering to the subject a pharmaceutically effective amount of a cancer immunotherapy agent. The step of administering the bispecific antibody and the step of administering the cancer immunotherapy agent may be performed sequentially in any order or may be performed simultaneously. The bispecific antibody and the cancer immunotherapy agent may be formulated and administered as a single preparation, or may be formulated separately and then administered simultaneously or sequentially in any order, but is not limited thereto. Furthermore, the cancer immunotherapy agent may be administered with the bispecific antibody in the form of a fusion protein or multi-specific antibody (e.g., a trispecific antibody or a tetraspecific antibody, etc.), but is not limited thereto.

Other embodiment provides a use of the bispecific antibody or the pharmaceutical composition in the prevention and/or treatment of cancer. Other embodiment provides a use of the bispecific antibody in preparing a pharmaceutical composition for the prevention and/or treatment of cancer.

Another embodiment provides a pharmaceutical composition for co-administration for the prevention and/or treatment of cancer comprising the bispecific antibody and the cancer immunotherapy agent as active ingredients. The pharmaceutical composition for co-administration may further comprise a pharmaceutically acceptable carrier. The bispecific antibody and the cancer immunotherapy agent may be included together in the form of a fusion protein or a multispecific antibody (e.g., a trispecific antibody or a tetraspecific antibody, etc.), or formulated for co-administration. In one embodiment, the bispecific antibody and the cancer immunotherapy agent may be formulated in a single preparation, or the bispecific antibody and the cancer immunotherapy agent may be formulated separately and then mixed, but are not limited thereto.

Another embodiment provides a method of preventing and/or treating cancer, comprising administering a pharmaceutically effective amount of the bispecific antibody and a pharmaceutically effective amount of the cancer immunotherapy agent to a subject in need thereof. In the method, the pharmaceutically effective amount of the bispecific antibody and the pharmaceutically effective amount of the cancer immunotherapy agent may be administered simultaneously or sequentially in any order. The bispecific antibody and the cancer immunotherapy agent may be formulated and administered as a single preparation, or may be formulated separately and then administered simultaneously or sequentially in any order, but is not limited thereto. Furthermore, the cancer immunotherapy agent may be administered with the bispecific antibody in the form of a fusion protein or multi-specific antibody (e.g., a trispecific antibody or a tetraspecific antibody, etc.), but is not limited thereto.

Other embodiment provides a use of the bispecific antibodies and the cancer immunotherapy agents in the prevention and/or treatment of cancer. Other embodiment provides a use of the bispecific antibodies and the cancer immunotherapy agents in preparing a pharmaceutical composition for the prevention and/or treatment of cancer. The bispecific antibody and the cancer immunotherapy agent may be included together in the form of a fusion protein or multi-specific antibody (e.g., a trispecific antibody or a tetraspecific antibody, etc.), or formulated for co-administration. In one example, the bispecific antibody and the cancer immunotherapy agent may be formulated in a single preparation, or the bispecific antibody and the cancer immunotherapy agent may be formulated separately and then mixed, but are not limited to.

As used herein, the cancer immunotherapy agent may be selected from any drug that exerts its anti-cancer effect by activating the immune system, e.g., by inhibiting immune checkpoints. For example, the cancer immunotherapy agent may be an agent (e.g., antibody, etc.) that inhibits the activity (e.g., interaction with a respective binding protein (e.g., ligand, etc.)) of one or more selected from the group consisting of PD-1, PD-L1, TIGIT, 4-1BB, OX40, CTLA-4, LAG-3, TIM-3, GITR, GITRL, ICOS, ICOSL, and VISTA, etc. In one embodiment, the cancer immunotherapy agent may be an agent that inhibits the interaction of PD-1 and PD-L1, such as, but not limited to, an agent that targets PD-1 (PD-1 inhibitor), an agent that targets PD-L1 (PD-L1 inhibitor), or a combination thereof. The agent that targets PD-1 may be, but not limited thereto, one or more selected from the group consisting of a protein (e.g., an antibody, an antigen-binding fragment of an antibody, an antibody analog, etc.), a nucleic acid molecule (e.g., an aptamer, siRNA, shRNA, microRNA, etc.), a small molecule compound, or the like, which binds (e.g., specifically binds) to PD-1. Furthermore, the agent targeting PD-L1 may be, but not limited thereto, one or more selected from the group consisting of a protein (e.g., an antibody, an antigen-binding fragment of an antibody, an antibody analog, etc.), a nucleic acid molecule (e.g., an aptamer, siRNA, shRNA, microRNA, etc.), a small molecule compound, or the like, which binds (e.g., specifically binds) to PD-L1.

In one embodiment, the cancer immunotherapy agent may be at least one selected from the group consisting of a PD-1 inhibitor (e.g., an anti-PD-1 antibody, an antigen-binding fragment thereof, etc.) and a PD-L1 inhibitor (e.g., an anti-PD-L1 antibody, an antigen-binding fragment thereof).

For example, the PD-1 inhibitor may be an anti-PD-1 antibody or an antigen-binding fragment thereof, and the anti-PD-1 antibody may be, but not limited to, one or more selected from the group consisting of pembrolizumab, nivolumab, cemiplimab, dostarlimab, and the like. The PD-L1 inhibitor may be an anti-PD-L1 antibody or an antigen-binding fragment thereof, and the anti-PD-L1 antibody may be, but not limited to, one or more selected from the group consisting of atezolizumab, durvalumab, avelumab, and the like.

The cancer may be a cancer characterized by low expression of HER2, expression of FcγRI, or both. For example, the cancer may be a HER2-low expressing cancer.

As used herein, the term “HER2-low expressing cancer” is not particularly limited as long as it is recognized as a HER2-low expressing cancer by a person skilled in the art, but can be broadly interpreted to include any case in which HER2 expression is below a certain level in the tumor as a whole. For example, it may mean expressing HER2 at a low level, having a low percentage of cells expressing HER2 within the tumor, or not expressing HER2 (HER2-naive). In another embodiment, “HER2-low expressing tumor (cancer)”, when interpreted narrowly, may be interpreted to mean, but is not limited to, expressing HER2 at a low level or having a low percentage of cells expressing HER2 within the tumor.

In one embodiment, a HER2-low expressing cancer may mean one or more of the following:
(1) an IHC score of 0, 1+, or 2+ as measured by immunohistochemistry using an anti-HER2 antibody specific for HER2;
(2) the level of HER2 in the tumor, as measured by conventional protein measurement methods, is 60% or less, 50% or less, 40% or less, 30% or less, or 20% or less relative to the HER2 level in the SK-BR-3 cell line (e.g., HTB-30 (ATCC)) (the lower limit may be 0% or a value greater than 0%);
(3) the level of HER2 in the tumor, as measured by conventional protein measurement methods, is lower than that in the HCC1954 cell line (e.g., CRL-2338 (ATCC)) (the lower limit may be zero or a value greater than zero); and
(4) the percentage of tumor cells expressing HER2 out of all tumor cells constituting the cancer, based on the number, the volume, or the weight of cells, is 50% or less, 45% or less, 40% or less, 35% or less, or 30% or less (the lower limit may be 0% or a number greater than 0%).

In another embodiment, a “HER2-low expressing cancer” may be (i) a cancer with HER2 expression determined to be 2+ by immunohistochemistry and negative for HER2 expression by in situ hybridization (ISH), or (ii) a cancer with HER2 expression determined to be 1+ by immunohistochemistry. ISH methods may include fluorescence in situ hybridization (FISH) or dual in situ hybridization (DISH). Any method of determining HER2 expression by immunohistochemistry, or any method of determining positive or negative HER2 expression by ISH, may be used without limitation as long as it is recognized by those skilled in the art. For example, any method of determining HER2 expression based on ASCO/CAP guideline, NCCN guideline, etc. may be used without limitation. In another embodiment, a “HER2-low expressing cancer” may be what would be recognized as a HER2-low expressing cancer by a person skilled in the art by Next Generation Sequencing (NGS), but is not limited thereto.

In one example, a HER2-low expressing cancer may mean that the percentage of tumor cells expressing HER2 out of all tumor cells constituting the cancer is, based on the number, the volume, or the weight of cells, 0.05 to 50%, 0.5 to 50%, 1 to 50%, 5 to 50%, 10 to 50%, 0.05 to 40%, 0.5 to 40%, 1 to 40%, 5 to 40%, 10 to 40%, 0.05 to 30%, 0.5 to 30%, 1 to 30%, 5 to 30%, or 10 to 30%.

The conventional protein measurement methods may be measurement by conventional enzymatic reactions, fluorescence, luminescence, and/or radiometric detection using compounds, antibodies, aptamers, or the like that specifically bind to the protein to be measured (e.g., HER2), and may include, but are not limited to, immunohistochemistry, ISH method, immunochromatography, enzyme-linked immunosorbent assay (ELISA), radioimmunoassay (RIA), enzyme immunoassay (EIA), fluorescence immunoassay (FIA), luminescence immunoassay (LIA), western blotting, microarray, and the like.

FcγRI refers to Fc gamma receptor l, which is also referred to as CD64. FcγRI-expressing cancer may refer to any type of cancer that contains cells expressing FcγRI (e.g., infiltrating immune cells within a tumor).

The anti-HER2/anti-4-1BB bispecific antibodies provided herein are characterized in that they have anti-cancer activity in high HER2 expressing cancers, as well as in HER2-low expressing cancers, and have superior 4-1BB activation and/or immune response induction activity in FcγRI expressing cancers, resulting in superior anti-cancer effects in cancers characterized by HER2-low expression and/or FcγRI expression.

A cancer that may be prevented and/or treated by the bispecific antibody or the pharmaceutical composition may be a cancer having HER2-low expression and/or FcγRI expression characteristics. The cancer may be selected from solid and hematologic cancers having HER2-low expression and/or FcγRI expression characteristics. The cancer may be that having HER2-low expression and/or FcγRI expression characteristics and being one or more selected from the group consisting of breast cancer, colon cancer, gastric cancer, lung cancer (e.g., squamous cell carcinoma of the lung, small cell lung cancer, non-small cell lung cancer, lung adenocarcinoma), peritoneal cancer, skin cancer, squamous cell carcinoma, melanoma of the skin or eye, rectal cancer, perianal cancer, esophageal cancer, small bowel tumor, endocrine gland cancer, parathyroid cancer, adrenal cancer, soft tissue sarcoma, urethral cancer, chronic or acute leukemia, lymphocytic lymphoma, liver cancer, gastrointestinal tract cancer, pancreatic cancer, glioblastoma, cervical cancer, ovarian cancer, bladder cancer, hepatocellular adenoma, endometrial or uterine cancer, salivary gland tumor, kidney cancer, prostate cancer, vulvar cancer, thyroid cancer, head and neck cancer, brain cancer, biliary tract cancer, gallbladder cancer, and the like, but not limited thereto. The cancer may be a primary cancer, a metastatic cancer, or a recurrent cancer.

As used herein, the term “preventing and/or treating cancer” may refer to killing cancer cells, inhibiting the proliferation of cancer cells, relieving symptoms associated with cancer, inhibiting the spread of cancer, or inhibiting the recurrence of cancer.

As used herein, the term “enhancement of an immune response” may mean, but is not limited to, 4-1BB signal activation, an enhancement or intensification of an immune response associated with 4-1BB, such as 4-1BB-induced signal activation (e.g., 4-1BB-induced NF-kB signal activation, increased cytokine release, target cell killing by immune cells such as T cells, etc.). In one embodiment, the enhancement of an immune response by the bispecific antibody provided herein can occur in the presence of HER2.

The pharmaceutical composition may further comprise a pharmaceutically acceptable carrier, diluent, and/or excipient in addition to the bispecific antibody as the active ingredient. The pharmaceutically acceptable carriers, diluents, and/or excipients may be any one selected from those commonly used in the formulation of antibodies. For example, pharmaceutically acceptable carriers include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, and mineral oil.

The pharmaceutical composition may further comprise one or more species selected from the group consisting of lubricants, wetting agents, sweeteners, flavor enhancers, emulsifiers, suspending agents, preservatives, and the like.

The bispecific antibody or pharmaceutical composition may be administered to a subject orally or parenterally. Parenteral administration may be intravenous, subcutaneous, intramuscular, intraperitoneal, endothelial, topical, intranasal, intrapulmonary, or rectal. Since oral administration leads to digestion of the protein or peptide, the active ingredient in the composition for oral administration may be coated or formulated to prevent digestion in the stomach. Additionally, the composition may be administered using any device that allows the active ingredient to be delivered to a target cell (e.g., a cancer cell).

As used herein, the term “pharmaceutically effective amount” may refer to an amount at which the active ingredient, bispecific antibody and/or cancer immunotherapy agent is capable of exerting a pharmaceutically meaningful effect in the prevention or treatment of cancer. The pharmaceutically effective amount of a bispecific antibody and/or cancer immunotherapy agent, or the appropriate dosage of a pharmaceutical composition expressed as an amount of bispecific antibody, may be prescribed in a variety of ways, depending on various factors, such as age, body weight, gender, pathologic conditions, diets, excretion speed, and/or response sensitivity of a patient, formulation types, time of administration, route of administration, methods of administration, and the like. For example, a pharmaceutically effective amount of a bispecific antibody (and/or cancer immunotherapy agent) or a suitable dosage of a pharmaceutical composition in an adult may be from about 0.001 to about 1000 mg (amount of bispecific antibody)/kg (body weight)/day, from about 0.01 to about 100 mg/kg/day, or from about 0.1 to about 50 mg/kg/day.

The subject to whom the bispecific antibody and/or cancer immunotherapy agent, or pharmaceutical composition is administered may be a mammal, such as, but not limited to, a human, monkey, rat, mouse, dog, cat, guinea pig, rabbit, rat, mouse, horse, bovine, cow, or the like, or cells or tissues obtained therefrom, and the subject may be the one suffering from cancer.

The pharmaceutical composition may be formulated with pharmaceutically acceptable carriers and/or excipients into a unit or multiple dosage forms by methods readily practicable by a person skilled in the art. The formulation may be an oil or aqueous medium, suspension, syrup, emulsion, extract, powder, granule, tablet or capsule, and may further comprise a dispersant or stabilizing agent.

### Advantageous Effects

The anti-HER2/anti-4-1BB bispecific antibodies provided herein are capable of activating 4-1BB signaling and enhancing the immune response against high HER2 expression cancers, as well as cancer cells with HER2-low expression and/or FcγRI expression characteristics. Thus, the bispecific antibody may be useful as a cancer immunotherapy agent that exhibits anticancer activity against cancers characterized by HER2-low expression and/or FcγRI expression.

### DESCRIPTION OF DRAWINGS

FIG. 1a is a graph showing the antigen (human 4-1BB) binding activities of anti-4-1BB antibodies as measured by ELISA.

FIG. 1b is a graph showing the binding activities of anti-4-1BB antibodies to 4-1BB expressing cells as measured by FACS.

FIG. 2a is a graph showing the antigen (human HER2) binding activities of anti-HER2/anti-4-1BB bispecific antibodies measured by ELISA.

FIG. 2b is a graph showing the antigen (human HER2) binding activities of anti-HER2/anti-4-1BB bispecific antibodies measured by ELISA.

FIG. 3a is a graph showing the antigen (human 4-1BB) binding activities of anti-HER2/anti-4-1BB bispecific antibodies as measured by ELISA.

FIG. 3b is a graph showing the antigen (human 4-1BB) binding activities of anti-HER2/anti-4-1BB bispecific antibodies as measured by ELISA.

FIG. 4 is a graph showing the efficacy of anti-HER2/anti-4-1BB bispecific antibodies in inducing 4-1BB activation in FcγRI expressing cells.

FIG. 5a is a graph showing the effect of anti-HER2/anti-4-1BB bispecific antibodies on Antibody Dependent Cellular Cytotoxicity (ADCC) in HER2-low expressing tumor cells (JIMT-1).

FIG. 5b is a graph showing the effect of anti-HER2/anti-4-1BB bispecific antibodies on Antibody Dependent Cellular Cytotoxicity (ADCC) in HER2-low expressing tumor cells (MDA-MB-231).

FIG. 6 is a graph showing the anti-cancer effect of anti-HER2/anti-4-1BB bispecific antibodies on HER2-low expressing tumors.

FIG. 7 is a graph showing the anti-cancer effect of co-administration of an anti-HER2/anti-4-1BB bispecific antibody and a PD-1 inhibitor on HER2-low expressing tumors.

### MODE FOR INVENTION

The present invention will now be described in detail with reference to the following examples.

The following embodiments are intended to illustrate the invention only and are not to be construed as limiting the invention.

### Example 1. Anti-4-1BB Antibodies

### 1.1 Preparation of Full Human Monoclonal Antibodies to 4-1BB

Full human monoclonal anti-4-1BB antibodies in full-length IgG form were screened by phage library (obtained from KBio Health) immunotube panning against 4-1BB. For panning of the phage library against the target molecule, a total of four pannings were performed using immunotubes coated with 4-1BB (NCBI Accession No. NP_001552.2).

Bacterial colonies from three pannings were grown in SB-carbenicillin (Biomatik cat #A2311-5g) in 96 deep-well plates until they become cloudy, at which point 10¹¹ pfu of VCSM13 helper phage (K-Bio Health) was added to each well. After infection at 37° C. for 1 hour with gentle shaking (80 rpm), 70 μg/mL of kanamycin was added and cells were incubated overnight at 30° C. with shaking at 200 rpm.

The next day, the plates were centrifuged and the supernatant containing phage was added to 4-1BB antigen-coated ELISA plates blocked with 3% (v/v) BSA (bovine serum albumin) in PBST (phosphate-buffered saline containing Tween 20). After 1 hour incubation at room temperature, the plate was washed three times with PBST and then anti-M13 antibody (Sino Biological cat #11973-MM05) was added. The plate was incubated for 1 hour, washed 3 times with PBST, and the binding activity was measured using tetramethylbenzidine (TMB).

The 4-1BB-specific binders were amplified for plasmid DNA sequencing. Light and heavy chain variable region (VL and VH) sequences were analyzed to identify unique sequences and determine sequence diversity as shown in Table 6 through Table 13 (underlined: CDR1, CDR2, and CDR3, in order).

TABLE 6

| 1A10 | Amino acid sequence (N′→C′) |
|---|---|
| Heavy Chain | EVQLLESGGGLVQPGGSLRLSCAASGFTFSSYDMSWVRQAPGKGLEWVSW |
| | ISYSGGSIYYADSVKGRFTISRDNSKNTLYLQMNSLRAEDTAVYYCARDG |
| | QRNSMREFDYWGQGTLVTVSSASTKGPSVFPLAPCSRSTSESTAALGCLV |
| | KDYFPEPVTVSWNSGALTSGVHTFPAVLQSSGLYSLSSVVTVPSSSLGTK |
| | TYTCNVDHKPSNTKVDKRVESKYGPPCPPCPAPEFLGGPSVFLFPPKPKD |
| | TLMISRTPEVTCVVVDVSQEDPEVQFNWYVDGVEVHNAKTKPREEQFNST |
| | YRVVSVLTVLHQDWLNGKEYKCKVSNKGLPSSIEKTISKAKGQPREPQVY |
| | TLPPSQEEMTKNQVSLTCLVKGFYPSDIAVEWESNGQPENNYKTTPPVLD |
| | SDGSFFLYSRLTVDKSRWQEGNVFSCSVMHEALHNHYTQKSLSLSLGK |
| | (SEQ ID NO: 56) |
| Heavy Chain | EVQLLESGGGLVQPGGSLRLSCAASGFTFSSYDMSWVRQAPGKGLEWVSW |
| Variable Region | ISYSGGSIYYADSVKGRFTISRDNSKNTLYLQMNSLRAEDTAVYYCARDG |
| (VH) | QRNSMREFDYWGQGTLVTVSS (SEQ ID NO: 18) |
| H-CDR1 | SYDMS (SEQ ID NO: 1) |
| H-CDR2 | WISYSGGSIYYADSVKG (SEQ ID NO: 4) |
| H-CDR3 | DGQRNSMREFDY (SEQ ID NO: 7) |
| Light Chain | QSVLTQPPSASGTPGRRVTISCSGSSSNIGNNYVTWYQQLPGTAPKLLIYA |
| | DSHRPSGVPDRFSGSKSGTSASLAISGLRSEDEADYYCATWDYSLSGYVFGG |
| | GTKLTVLGQPKAAPSVTLFPPSSEELQANKATLVCLISDFYPGAVTVAWKAD |
| | SSPVKAGVETTTPSKQSNNKYAASSYLSLTPEQWKSHRSYSCQVTHEGSTV |
| | EKTVAPAECS (SEQ ID NO: 62) |
| Light Chain | QSVLTQPPSASGTPGRRVTISCSGSSSNIGNNYVTWYQQLPGTAPKLLIYAD |
| Variable Region | SHRPSGVPDRFSGSKSGTSASLAISGLRSEDEADYYCATWDYSLSGYVFGG |
| (VL) | GTKLTVL (SEQ ID NO: 30) |
| L-CDR1 | SGSSSNIGNNYVT (SEQ ID NO: 12) |
| L-CDR2 | ADSHRPS (SEQ ID NO: 14) |
| L-CDR3 | ATWDYSLSGYV (SEQ ID NO: 16) |

TABLE 7

| 1A10 M4 | Amino acid sequence (N′→C′) |
|---|---|
| Heavy Chain | EVQLLESGGGLVQPGGSLRLSCAASGFTFSSYDMSWVRQAPGKGLEWVSW |
| | ISYSGGSIYYADSVKGRFTISRDNSKNTLYLQMNSLRAEDTAVYYCARDAQ |
| | RNSMREFDYWGQGTLVTVSSASTKGPSVFPLAPCSRSTSESTAALGCLVKD |
| | YFPEPVTVSWNSGALTSGVHTFPAVLQSSGLYSLSSVVTVPSSSLGTKTYT |
| | CNVDHKPSNTKVDKRVESKYGPPCPPCPAPEFLGGPSVFLFPPKPKDTLMI |
| | SRTPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSDGSFFL |
| | YSRLTVDKSRWQEGNVFSCSVMHEALHNHYTQKSLSLSLGK |
| | (SEQ ID NO: 57) |
| Heavy Chain | EVQLLESGGGLVQPGGSLRLSCAASGFTFSSYDMSWVRQAPGKGLEWVSW |
| Variable Region | ISYSGGSIYYADSVKGRFTISRDNSKNTLYLQMNSLRAEDTAVYYCARDA |
| (VH) | QRNSMREFDYWGQGTLVTVSS (SEQ ID NO: 19) |
| H-CDR1 | SYDMS (SEQ ID NO: 1) |
| H-CDR2 | WISYSGGSIYYADSVKG (SEQ ID NO: 4) |
| H-CDR3 | DAQRNSMREFDY (SEQ ID NO: 8) |
| Light Chain | QSVLTQPPSASGTPGRRVTISCSGSSSNIGNNYVTWYQQLPGTAPKLLIYA |
| | DSHRPSGVPDRFSGSKSGTSASLAISGLRSEDEADYYCATWDYSLSGYVFG |
| | GGTKLTVLGQPKAAPSVTLFPPSSEELQANKATLVCLISDFYPGAVTVAWK |
| | ADSSPVKAGVETTTPSKQSNNKYAASSYLSLTPEQWKSHRSYSCQVTHEGS |
| | TVEKTVAPAECS (SEQ ID NO: 62) |
| Light Chain | QSVLTQPPSASGTPGRRVTISCSGSSSNIGNNYVTWYQQLPGTAPKLLIYAD |
| Variable Region | SHRPSGVPDRFSGSKSGTSASLAISGLRSEDEADYYCATWDYSLSGYVFGG |
| (VL) | GTKLTVL (SEQ ID NO: 30) |
| L-CDR1 | SGSSSNIGNNYVT (SEQ ID NO: 12) |
| L-CDR2 | ADSHRPS (SEQ ID NO: 14) |
| L-CDR3 | ATWDYSLSGYV (SEQ ID NO: 16) |

TABLE 8

| 1A10 M11 | Amino acid sequence (N′→C′) |
|---|---|
| Heavy Chain | EVQLLESGGGLVQPGGSLRLSCAASGFTFSSYDMSWVRQAPGKGLEWVSW |
| | ISYSGGSIYYADSVKGRFTISRDNSKNTLYLQMNSLRAEDTAVYYCARDAQ |
| | RQSMREFDYWGQGTLVTVSSASTKGPSVFPLAPCSRSTSESTAALGCLVKD |
| | YFPEPVTVSWNSGALTSGVHTFPAVLQSSGLYSLSSVVTVPSSSLGTKTYT |
| | CNVDHKPSNTKVDKRVESKYGPPCPPCPAPEFLGGPSVFLFPPKPKDTLMI |
| | SRTPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSDGSFFL |
| | YSRLTVDKSRWQEGNVFSCSVMHEALHNHYTQKSLSLSLGK |
| | (SEQ ID NO: 58) |
| Heavy Chain | EVQLLESGGGLVQPGGSLRLSCAASGFTFSSYDMSWVRQAPGKGLEWVSW |
| Variable Region | ISYSGGSIYYADSVKGRFTISRDNSKNTLYLQMNSLRAEDTAVYYCARDA |
| (VH) | QRQSMREFDYWGQGTLVTVSS (SEQ ID NO: 20) |
| H-CDR1 | SYDMS (SEQ ID NO: 1) |
| H-CDR2 | WISYSGGSIYYADSVKG (SEQ ID NO: 4) |
| H-CDR3 | DAQRQSMREFDY (SEQ ID NO: 9) |
| Light Chain | QSVLTQPPSASGTPGRRVTISCSGSSSNIGNNYVTWYQQLPGTAPKLLIYAD |
| | SHRPSGVPDRFSGSKSGTSASLAISGLRSEDEADYYCATWDYSLSGYVFGGG |
| | TKLTVLGQPKAAPSVTLFPPSSEELQANKATLVCLISDFYPGAVTVAWKADS |
| | SPVKAGVETTTPSKQSNNKYAASSYLSLTPEQWKSHRSYSCQVTHEGSTVEK |
| | TVAPAECS (SEQ ID NO: 62) |
| Light Chain | QSVLTQPPSASGTPGRRVTISCSGSSSNIGNNYVTWYQQLPGTAPKLLIYAD |
| Variable Region | SHRPSGVPDRFSGSKSGTSASLAISGLRSEDEADYYCATWDYSLSGYVFGG |
| (VL) | GTKLTVL (SEQ ID NO: 30) |
| L-CDR1 | SGSSSNIGNNYVT (SEQ ID NO: 12) |
| L-CDR2 | ADSHRPS (SEQ ID NO: 14) |
| L-CDR3 | ATWDYSLSGYV (SEQ ID NO: 16) |

TABLE 9

| 1A10 M12 | Amino acid sequence (N′→C′) |
|---|---|
| Heavy Chain | EVQLLESGGGLVQPGGSLRLSCAASGFTFSSYDMSWVRQAPGKGLEWVS |
| | WISYSGGSIYYADSVKGRFTISRDNSKNTLYLQMNSLRAEDTAVYYCARDAQ |
| | RNSMREFDYWGQGTLVTVSSASTKGPSVFPLAPCSRSTSESTAALGCLVKD |
| | YFPEPVTVSWNSGALTSGVHTFPAVLQSSGLYSLSSVVTVPSSSLGTKTYTC |
| | NVDHKPSNTKVDKRVESKYGPPCPPCPAPEFLGGPSVFLFPPKPKDTLMIS |
| | RTPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQE |
| | EMTKNQVSLTCLVKGFYPSDIAVEWESNGQPENNYKTTPPVLDSDGSFFLY |
| | SRLTVDKSRWQEGNVFSCSVMHEALHNHYTQKSLSLSLGK (SEQ ID NO: |
| | 57) |
| Heavy Chain | EVQLLESGGGLVQPGGSLRLSCAASGFTFSSYDMSWVRQAPGKGLEWVS |
| Variable Region | WISYSGGSIYYADSVKGRFTISRDNSKNTLYLQMNSLRAEDTAVYYCARDAQ |
| (VH) | RNSMREFDYWGQGTLVTVSS (SEQ ID NO: 19) |
| H-CDR1 | SYDMS (SEQ ID NO: 1) |
| H-CDR2 | WISYSGGSIYYADSVKG (SEQ ID NO: 4) |
| H-CDR3 | DAQRNSMREFDY (SEQ ID NO: 8) |
| Light Chain | QSVLTQPPSASGTPGQRVTISCSGSSSNIGNNYVTWYQQLPGTAPKLLIYAD |
| | SHRPSGVPDRFSGSKSGTSASLAISGLRSEDEADYYCATWDYSLSGYVFGG |
| | GTKLTVLGQPKAAPSVTLFPPSSEELQANKATLVCLISDFYPGAVTVAWKAD |
| | SSPVKAGVETTTPSKQSNNKYAASSYLSLTPEQWKSHRSYSCQVTHEGSTV |
| | EKTVAPAECS (SEQ ID NO: 63) |
| Light Chain | QSVLTQPPSASGTPGQRVTISCSGSSSNIGNNYVTWYQQLPGTAPKLLIYAD |
| Variable Region | SHRPSGVPDRFSGSKSGTSASLAISGLRSEDEADYYCATWDYSLSGYVFGG |
| (VL) | GTKLTVL (SEQ ID NO: 31) |
| L-CDR1 | SGSSSNIGNNYVT (SEQ ID NO: 12) |
| L-CDR2 | ADSHRPS (SEQ ID NO: 14) |
| L-CDR3 | ATWDYSLSGYV (SEQ ID NO: 16) |

TABLE 10

| 1A10 M13 | Amino acid sequence (N′→C′) |
|---|---|
| Heavy Chain | EVQLLESGGGLVQPGGSLRLSCAASGFTFSSYDMSWVRQAPGKGLEWVS |
| | WISYSGGSIYYADSVKGRFTISRDNSKNTLYLQMNSLRAEDTAVYYCARDAQ |
| | RQSMREFDYWGQGTLVTVSSASTKGPSVFPLAPCSRSTSESTAALGCLVKD |
| | YFPEPVTVSWNSGALTSGVHTFPAVLQSSGLYSLSSVVTVPSSSLGTKTYTC |
| | NVDHKPSNTKVDKRVESKYGPPCPPCPAPEFLGGPSVFLFPPKPKDTLMIS |
| | RTPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQE |
| | EMTKNQVSLTCLVKGFYPSDIAVEWESNGQPENNYKTTPPVLDSDGSFFLY |
| | SRLTVDKSRWQEGNVFSCSVMHEALHNHYTQKSLSLSLGK (SEQ ID NO: |
| | 58) |
| Heavy Chain | EVQLLESGGGLVQPGGSLRLSCAASGFTFSSYDMSWVRQAPGKGLEWVS |
| Variable Region | WISYSGGSIYYADSVKGRFTISRDNSKNTLYLQMNSLRAEDTAVYYCARDAQ |
| (VH) | RQSMREFDYWGQGTLVTVSS (SEQ ID NO: 20) |
| H-CDR1 | SYDMS (SEQ ID NO: 1) |
| H-CDR2 | WISYSGGSIYYADSVKG (SEQ ID NO: 4) |
| H-CDR3 | DAQRQSMREFDY (SEQ ID NO: 9) |
| Light Chain | QSVLTQPPSASGTPGQRVTISCSGSSSNIGNNYVTWYQQLPGTAPKLLIYAD |
| | SHRPSGVPDRFSGSKSGTSASLAISGLRSEDEADYYCATWDYSLSGYVFGG |
| | GTKLTVLGQPKAAPSVTLFPPSSEELQANKATLVCLISDFYPGAVTVAWKAD |
| | SSPVKAGVETTTPSKQSNNKYAASSYLSLTPEQWKSHRSYSCQVTHEGSTV |
| | EKTVAPAECS (SEQ ID NO: 63) |
| Light Chain | QSVLTQPPSASGTPGQRVTISCSGSSSNIGNNYVTWYQQLPGTAPKLLIYAD |
| Variable Region | SHRPSGVPDRFSGSKSGTSASLAISGLRSEDEADYYCATWDYSLSGYVFGG |
| (VL) | GTKLTVL (SEQ ID NO: 31) |
| L-CDR1 | SGSSSNIGNNYVT (SEQ ID NO: 12) |
| L-CDR2 | ADSHRPS (SEQ ID NO: 14) |
| L-CDR3 | ATWDYSLSGYV (SEQ ID NO: 16) |

TABLE 11

| 1A12 | Amino acid sequence (N′→C′) |
|---|---|
| Heavy Chain | EVQLLESGGGLVQPGGSLRLSCAASGFTFSGYDMSWVRQAPGKGLEWVS |
| | VIYPDDGNTYYADSVKGRFTISRDNSKNTLYLQMNSLRAEDAAVYYCAKHG |
| | GQKPTTKSSSAYGMDGWGQGTLVTVSSASTKGPSVFPLAPCSRSTSESTA |
| | ALGCLVKDYFPEPVTVSWNSGALTSGVHTFPAVLQSSGLYSLSSVVTVPSS |
| | SLGTKTYTCNVDHKPSNTKVDKRVESKYGPPCPPCPAPEFLGGPSVFLFPP |
| | KPKDTLMISRTPEVTCVVVDVSQEDPEVQFNWYVDGVEVHNAKTKPREEQF |
| | NSTYRVVSVLTVLHQDWLNGKEYKCKVSNKGLPSSIEKTISKAKGQPREPQV |
| | YTLPPSQEEMTKNQVSLTCLVKGFYPSDIAVEWESNGQPENNYKTTPPVLD |
| | SDGSFFLYSRLTVDKSRWQEGNVFSCSVMHEALHNHYTQKSLSLSLGK |
| | (SEQ ID NO: 59) |
| Heavy Chain | EVQLLESGGGLVQPGGSLRLSCAASGFTFSGYDMSWVRQAPGKGLEWVS |
| Variable Region | VIYPDDGNTYYADSVKGRFTISRDNSKNTLYLQMNSLRAEDAAVYYCAKHG |
| (VH) | GQKPTTKSSSAYGMDGWGQGTLVTVSS (SEQ ID NO: 21) |
| H-CDR1 | GYDMS (SEQ ID NO: 2) |
| H-CDR2 | VIYPDDGNTYYADSVKG (SEQ ID NO: 5) |
| H-CDR3 | HGGQKPTTKSSSAYGMDG (SEQ ID NO: 10) |
| Light Chain | QSVLTQPPSASGTPGRRVTISCSGSSSNIGNNYVTWYQQLPGTAPKLLIYAD |
| | SHRPSGVPDRFSGSKSGTSASLAISGLRSEDEADYYCATWDYSLSGYVFGG |
| | GTKLTVLGQPKAAPSVTLFPPSSEELQANKATLVCLISDFYPGAVTVAWKAD |
| | SSPVKAGVETTTPSKQSNNKYAASSYLSLTPEQWKSHRSYSCQVTHEGSTV |
| | EKTVAPAECS (SEQ ID NO: 62) |
| Light Chain | QSVLTQPPSASGTPGRRVTISCSGSSSNIGNNYVTWYQQLPGTAPKLLIYAD |
| Variable Region | SHRPSGVPDRFSGSKSGTSASLAISGLRSEDEADYYCATWDYSLSGYVFGG |
| (VL) | GTKLTVL (SEQ ID NO: 30) |
| L-CDR1 | SGSSSNIGNNYVT (SEQ ID NO: 12) |
| L-CDR2 | ADSHRPS (SEQ ID NO: 14) |
| L-CDR3 | ATWDYSLSGYV (SEQ ID NO: 16) |

TABLE 12

| 1A12 M1 | Amino acid sequence (N′→C′) |
|---|---|
| Heavy Chain | EVQLLESGGGLVQPGGSLRLSCAASGFTFSGYDMSWVRQAPGKGLEWVS |
| | VIYPDDGNTYYADSVKGRFTISRDNSKNTLYLQMNSLRAEDTAVYYCAKHG |
| | GQKPTTKSSSAYGMDGWGQGTLVTVSSASTKGPSVFPLAPCSRSTSESTA |
| | ALGCLVKDYFPEPVTVSWNSGALTSGVHTFPAVLQSSGLYSLSSVVTVPSS |
| | SLGTKTYTCNVDHKPSNTKVDKRVESKYGPPCPPCPAPEFLGGPSVFLFPP |
| | KPKDTLMISRTPEVTCVVVDVSQEDPEVQFNWYVDGVEVHNAKTKPREEQF |
| | NSTYRVVSVLTVLHQDWLNGKEYKCKVSNKGLPSSIEKTISKAKGQPREPQV |
| | YTLPPSQEEMTKNQVSLTCLVKGFYPSDIAVEWESNGQPENNYKTTPPVLD |
| | SDGSFFLYSRLTVDKSRWQEGNVFSCSVMHEALHNHYTQKSLSLSLGK |
| | (SEQ ID NO: 60) |
| Heavy Chain | EVQLLESGGGLVQPGGSLRLSCAASGFTFSGYDMSWVRQAPGKGLEWVS |
| Variable Region | VIYPDDGNTYYADSVKGRFTISRDNSKNTLYLQMNSLRAEDTAVYYCAKHG |
| (VH) | GQKPTTKSSSAYGMDGWGQGTLVTVSS (SEQ ID NO: 22) |
| H-CDR1 | GYDMS (SEQ ID NO: 2) |
| H-CDR2 | VIYPDDGNTYYADSVKG (SEQ ID NO: 5) |
| H-CDR3 | HGGQKPTTKSSSAYGMDG (SEQ ID NO: 10) |
| Light Chain | QSVLTQPPSASGTPGQRVTISCSGSSSNIGNNYVTWYQQLPGTAPKLLIYAD |
| | SHRPSGVPDRFSGSKSGTSASLAISGLRSEDEADYYCATWDYSLSGYVFGG |
| | GTKLTVLGQPKAAPSVTLFPPSSEELQANKATLVCLISDFYPGAVTVAWKAD |
| | SSPVKAGVETTTPSKQSNNKYAASSYLSLTPEQWKSHRSYSCQVTHEGSTV |
| | EKTVAPAECS (SEQ ID NO: 63) |
| Light Chain | QSVLTQPPSASGTPGQRVTISCSGSSSNIGNNYVTWYQQLPGTAPKLLIYAD |
| Variable Region | SHRPSGVPDRFSGSKSGTSASLAISGLRSEDEADYYCATWDYSLSGYVFGG |
| (VL) | GTKLTVL |
| | (SEQ ID NO: 31) |
| L-CDR1 | SGSSSNIGNNYVT (SEQ ID NO: 12) |
| L-CDR2 | ADSHRPS (SEQ ID NO: 14) |
| L-CDR3 | ATWDYSLSGYV (SEQ ID NO: 16) |

TABLE 13

| AB41 | Amino acid sequence (N′→C′) |
|---|---|
| Heavy Chain | QVQLQQSGAEVIKPGASVKLSCKASGYTFSSYWMHWVRQAPGQGLEWIGE |
| | INPGNGHTNYNEKFKSRATLTGDTSTSTVYMELSSLRSEDTAVYYCARSFTT |
| | ARAFAYWGQGTLVTVSSASTKGPSVFPLAPCSRSTSESTAALGCLVKDYFP |
| | EPVTVSWNSGALTSGVHTFPAVLQSSGLYSLSSVVTVPSSSLGTKTYTCNV |
| | DHKPSNTKVDKRVESKYGPPCPPCPAPEFLGGPSVFLFPPKPKDTLMISRTP |
| | EVTCVVVDVSQEDPEVQFNWYVDGVEVHNAKTKPREEQFNSTYRVVSVLT |
| | VLHQDWLNGKEYKCKVSNKGLPSSIEKTISKAKGQPREPQVYTLPPSQEEM |
| | TKNQVSLTCLVKGFYPSDIAVEWESNGQPENNYKTTPPVLDSDGSFFLYSRL |
| | TVDKSRWQEGNVFSCSVMHEALHNHYTQKSLSLSLGK (SEQ ID NO: 61) |
| Heavy Chain | QVQLQQSGAEVIKPGASVKLSCKASGYTFSSYWMHWVRQAPGQGLEWIGE |
| Variable Region | INPGNGHTNYNEKFKSRATLTGDTSTSTVYMELSSLRSEDTAVYYCARSFTT |
| (VH) | ARAFAYWGQGTLVTVSS (SEQ ID NO: 23) |
| H-CDR1 | SYWMH (SEQ ID NO: 3) |
| H-CDR2 | EINPGNGHTNYNEKFKS (SEQ ID NO: 6) |
| H-CDR3 | SFTTARAFAY (SEQ ID NO: 11) |
| Light Chain | DIVMTQSPAFLSVTPGEKVTITCRASQTISDYLHWYQQKPDQAPKLLIKYASQ |
| | SISGIPSRFSGSGSGTDFTFTISSLEAEDAATYYCQDGHSFPPTFGQGTKLEI |
| | KRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKSF |
| | NRGEC (SEQ ID NO: 64) |
| Light Chain | DIVMTQSPAFLSVTPGEKVTITCRASQTISDYLHWYQQKPDQAPKLLIKYASQ |
| Variable Region | SISGIPSRFSGSGSGTDFTFTISSLEAEDAATYYCQDGHSFPPTFGQGTKLEI |
| (VL) | KR (SEQ ID NO: 32) |
| L-CDR1 | RASQTISDYLH (SEQ ID NO: 13) |
| L-CDR2 | YASQSIS (SEQ ID NO: 15) |
| L-CDR3 | QDGHSFPPT (SEQ ID NO: 17) |

1.2. Preparation of scFv Antibodies Against 4-1BB

The anti-4-1BB scFv antibodies having the structure of (N′)-VL-linker-VH-(C′) were prepared using the variable regions of the full human monoclonal antibodies to 4-1BB shown in Tables 6 through 13 of Example 1.1. The 44th amino acid residue “G” in the heavy chain variable region is substituted with “C” and the 103rd amino acid residue “G” in the light chain variable region is substituted with “C.” These amino acid substitutions from “G” to “C” in scFv may contribute to the increased stability of bispecific antibodies containing scFv as one target-specific portion. The amino acid sequences of the prepared anti-4-1BB scFv are shown in Tables 14 to 19 below, and those skilled in the art will recognize that changes or modifications in the amino acid sequence can be made to meet specific purposes, including applying various types of peptide linkers such as (GGGGS)2 (SEQ ID NO: 90), (GGGGS)3 (SEQ ID NO: 85), (GGGGS)4 (SEQ ID NO: 87), or (GS)9 (SEQ ID NO: 86) in the embodiments below.

TABLE 14

| 1A10 (scFv) | Amino acid sequence (N′→C′) |
|---|---|
| Light chain | QSVLTQPPSASGTPGRRVTISCSGSSS |
| variable | NIGNNYVTWYQQLPGTAPKLLIYADSH |
| region | RPSGVPDRFSGSKSGTSASLAISGLRS |
| (VL) | EDEADYYCATWDYSLSGYVFGCGTKLT |
| | VL (SEQ ID NO: 33) |
| Linker | GGGGSGGGGSGGGGSGGGGS |
| | (SEQ ID NO: 87) |
| Heavy chain | EVQLLESGGGLVQPGGSLRLSCAASGF |
| variable | TFSSYDMSWVRQAPGKCLEWVSWISYS |
| region | GGSIYYADSVKGRFTISRDNSKNTLYL |
| (VH) | QMNSLRAEDTAVYYCARDGQRNSMREF |
| | DYWGQGTLVTVSS (SEQ ID NO: 24) |

TABLE 15

| 1A10 M4 (scFv) | Amino acid sequence (N′→C′) |
|---|---|
| Light chain | QSVLTQPPSASGTPGRRVTISCSGSSSNIGNNYVTWYQQLPGTAPKLLIYA |
| variable region | DSHRPSGVPDRFSGSKSGTSASLAISGLRSEDEADYYCATWDYSLSGYVF |
| (VL) | GCGTKLTVL (SEQ ID NO: 33) |
| Linker | GGGGSGGGGSGGGGSGGGGS (SEQ ID NO: 87) |
| Heavy chain | EVQLLESGGGLVQPGGSLRLSCAASGFTFSSYDMSWVRQAPGKCLEWVS |
| variable region | WISYSGGSIYYADSVKGRFTISRDNSKNTLYLQMNSLRAEDTAVYYCARDA |
| (VH) | QRNSMREFDYWGQGTLVTVSS (SEQ ID NO: 25) |

TABLE 16

| 1A10 M12 (scFv) | Amino acid sequence (N′→C′) |
|---|---|
| Light chain | QSVLTQPPSASGTPGQRVTISCSGSSSNIGNNYVTWYQQLPGTAPKLLIYA |
| variable region | DSHRPSGVPDRFSGSKSGTSASLAISGLRSEDEADYYCATWDYSLSGYVF |
| (VL) | GCGTKLTVL (SEQ ID NO: 34) |
| Linker | GGGGSGGGGSGGGGSGGGGS (SEQ ID NO: 87) |
| Heavy chain | EVQLLESGGGLVQPGGSLRLSCAASGFTFSSYDMSWVRQAPGKCLEWVS |
| variable region | WISYSGGSIYYADSVKGRFTISRDNSKNTLYLQMNSLRAEDTAVYYCARDA |
| (VH) | QRNSMREFDYWGQGTLVTVSS (SEQ ID NO: 25) |

TABLE 17

| 1A12 (scFv) | Amino acid sequence (N′>C′) |
|---|---|
| Light chain | QSVLTQPPSASGTPGRRVTISCSGSSSNIGNNYVTWYQQLPGTAPKLLIYA |
| variable region | DSHRPSGVPDRFSGSKSGTSASLAISGLRSEDEADYYCATWDYSLSGYVF |
| (VL) | GCGTKLTVL (SEQ ID NO: 33) |
| Linker | GGGGSGGGGSGGGGSGGGGS (SEQ ID NO: 87) |
| Heavy chain | EVQLLESGGGLVQPGGSLRLSCAASGFTFSGYDMSWVRQAPGKCLEWVS |
| variable region | VIYPDDGNTYYADSVKGRFTISRDNSKNTLYLQMNSLRAEDAAVYYCAKHG |
| (VH) | GQKPTTKSSSAYGMDGWGQGTLVTVSS (SEQ ID NO: 27) |

TABLE 18

| 1A12 M1 (scFv) | Amino acid sequence (N′→C′) |
|---|---|
| Light chain | QSVLTQPPSASGTPGQRVTISCSGSSSNIGNNYVTWYQQLPGTAPKLLIYA |
| variable region | DSHRPSGVPDRFSGSKSGTSASLAISGLRSEDEADYYCATWDYSLSGYVF |
| (VL) | GCGTKLTVL (SEQ ID NO: 34) |
| Linker | GGGGSGGGGSGGGGSGGGGS (SEQ ID NO: 87) |
| Heavy chain | EVQLLESGGGLVQPGGSLRLSCAASGFTFSGYDMSWVRQAPGKCLEWVS |
| variable region | VIYPDDGNTYYADSVKGRFTISRDNSKNTLYLQMNSLRAEDTAVYYCAKHG |
| (VH) | GQKPTTKSSSAYGMDGWGQGTLVTVSS (SEQ ID NO: 28) |

TABLE 19

| AB41 (scFv) | Amino acid sequence (N′→C′) |
|---|---|
| Light chain | DIVMTQSPAFLSVTPGEKVTITCRASQTISDYLHWYQQKPDQAPKLLIKYAS |
| variable region | QSISGIPSRFSGSGSGTDFTFTISSLEAEDAATYYCQDGHSFPPTFGCGTKL |
| (VL) | EIKR (SEQ ID NO: 88) |
| Linker | GGGGSGGGGSGGGGSGGGGS (SEQ ID NO: 87) |
| Heavy chain | QVQLQQSGAEVIKPGASVKLSCKASGYTFSSYWMHWVRQAPGQCLEWIG |
| variable region | EINPGNGHTNYNEKFKSRATLTGDTSTSTVYMELSSLRSEDTAVYYCARSF |
| (VH) | TTARAFAYWGQGTLVTVSS (SEQ ID NO: 29) |

### 1.3. Antigen-Binding Capacity of Anti-4-1BB Antibodies (Full-Length IgG Form) Against Human 4-1BB

### (1) Antigen Binding Activity Measured by ELISA

To evaluate the antigen binding activity, the antibody candidates prepared in Example 1.1 were tested by ELISA. Briefly, microtiter plates were coated with 0.1 μg/ml of human 4-1BB-Fc protein (Sino Biological) in PBS, incubated at 100 μl/well overnight at 4° C., and blocked with 100 μl/well of 5% (v/v) BSA.

A 5-fold dilution of humanized antibodies (1A10, 1A12, and AB41) starting at 10 μg/ml was added to each well and incubated for 1 to 2 hours at room temperature (RT). Plates were washed with PBS/Tween and then incubated with goat anti-human IgG antibody conjugated with Horse Radish Peroxidase (HRP) (Thermo) for 1 hour at room temperature. After washing, the plates were developed with TMB substrate and analyzed spectrophotometrically at OD 450-630 nm.

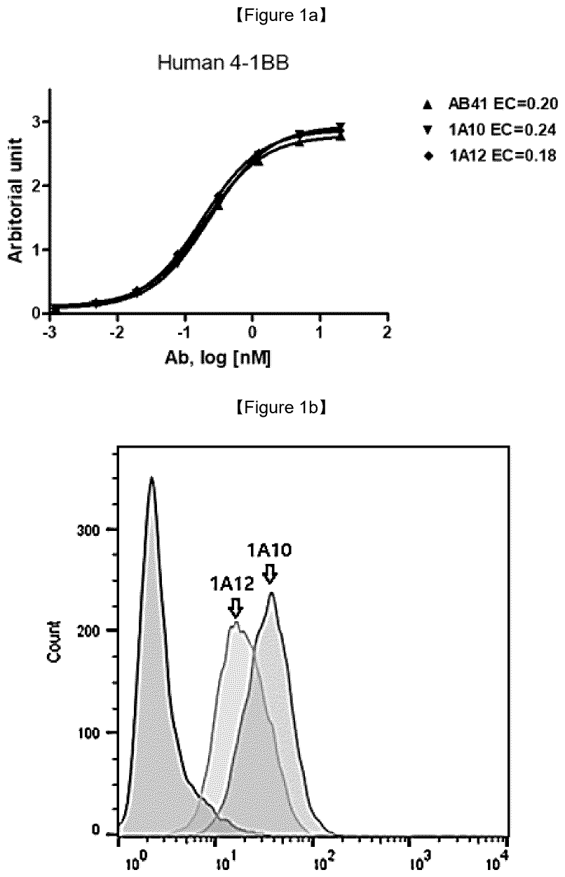

As a result, as shown in FIG. 1a, all tested anti-4-1BB antibodies exhibited 4-1BB binding activity.

### (2) Cell Binding Activity Measured by FACS

To assess cell-binding activity, the binding of the antibody candidates to human 4-1BB was analyzed by fluorescence-activated cell sorting (FACS). Briefly, GloResponse™ NFκB-luc2/4-1BB Jurkat cell line (Promega; 3×10⁵ of cells) expressing human 4-1BB on the surface was incubated with antibodies (1A10 and 1A12, 10 μg/mL each). After washing with FACS buffer (1% (v/v) BSA in PBS), FITC-anti-human IgG antibody (Sigma, F9512, concentration: 2.0 mg/ml) was added to each well and incubated for 1 hr at 4° C. The mean fluorescence intensity (MFI) of FITC was measured by FACSCalibur (BD Biosciences).

As a result, as shown in FIG. 1b, all tested anti-4-1BB antibodies exhibited efficient binding activity to 4-1BB expressed on the human cell surface.

### Example 2. Preparation of Anti-HER2 Antibodies

As HER2 targeting moieties for anti-HER2/anti-4-1BB bispecific antibodies, trastuzumab (Genentech; hereafter “HER2(WT)”, DrugBank Accession No. DB00072; human IgG1 Kappa monoclonal antibody) or its antigen-binding fragments such as scFv were used.

The sequences of HER2(WT) are summarized in Table 20 below.

The constant region of the anti-HER2 antibody contained in the bispecific antibody can be modified by introducing one or more mutations or changes in the human IgG1. One exemplary embodiment, HER2 (NA or N297A), is shown in Table 20 below.

TABLE 20

| HER2(WT) | Amino acid sequence (N′→C′) |
|---|---|
| Heavy Chain | EVQLVESGGGLVQPGGSLRLSCAASGFNIKDTYIHWVRQAPGKGLEWVARIY |
| | PTNGYTRYADSVKGRFTISADTSKNTAYLQMNSLRAEDTAVYYCSRWGGDGF |
| | YAMDYWGQGTLVTVSSASTKGPSVFPLAPSSKSTSGGTAALGCLVKDYFPEP |
| | VTVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSREEMTKNQ |
| | VSLTCLVKGFYPSDIAVEWESNGQPENNYKTTPPVLDSDGSFFLYSKLTVDKS |
| | RWQQGNVFSCSVMHEALHNHYTQKSLSLSPGK (SEQ ID NO: 73) |
| Light Chain | DIQMTQSPSSLSASVGDRVTITCRASQDVNTAVAWYQQKPGKAPKLLIYSASF |
| | LYSGVPSRFSGSRSGTDFTLTISSLQPEDFATYYCQQHYTTPPTFGQGTKVEI |
| | KRTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSFNR |
| | GEC (SEQ ID NO: 75) |
| HER2(NA) | |
| (N297A) | Amino acid sequence (N′→C′) |
| Heavy Chain | EVQLVESGGGLVQPGGSLRLSCAASGFNIKDTYIHWVRQAPGKGLEWVARIY |
| | PTNGYTRYADSVKGRFTISADTSKNTAYLQMNSLRAEDTAVYYCSRWGGDGF |
| | YAMDYWGQGTLVTVSSASTKGPSVFPLAPSSKSTSGGTAALGCLVKDYFPEP |
| | VTVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPEV |
| | TCVVVDVSHEDPEVKFNWYVDGVEVHNAKTKPREEQYASTYRVVSVLTVLH |
| | QDWLNGKEYKCKVSNKALPAPIEKTISKAKGQPREPQVYTLPPSREEMTKNQ |
| | VSLTCLVKGFYPSDIAVEWESNGQPENNYKTTPPVLDSDGSFFLYSKLTVDKS |
| | RWQQGNVFSCSVMHEALHNHYTQKSLSLSPGK (SEQ ID NO: 74) |
| Light Chain | DIQMTQSPSSLSASVGDRVTITCRASQDVNTAVAWYQQKPGKAPKLLIYSASF |
| | LYSGVPSRFSGSRSGTDFTLTISSLQPEDFATYYCQQHYTTPPTFGQGTKVEI |
| | KRTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSFNR |
| | GEC (SEQ ID NO: 75) |

### Example 3. Preparation of Anti-HER2/Anti-4-1BB Bispecific Antibodies

Various anti-HER2/anti-4-1BB bispecific antibody candidates were prepared in either full-length IgG (anti-HER2 antibody)-scFv (anti-4-1BB antibody) format or full-length IgG (anti-4-1BB antibody)-scFv (anti-HER2 antibody) format. In this embodiment, the anti-HER2 IgG and 4-1BB scFv clones prepared in Example 2 and Example 1.2, respectively, were exemplarily selected to prepare an anti-HER2/anti-4-1BB bispecific antibody in the form of an IgG-scFv fusion (an scFv antibody fragment of one antigen is fused to the C-terminus of an IgG of the other antigen). IgG1 with a mutant backbone with reduced ADCC (N297A mutation; Cancer Cell, vol. 19, issue 1, pp. 101-113, etc.) was used when HER2 targeting moiety was located in the whole IgG portion, and IgG4 was used when 4-1BB targeting moiety was located in the whole IgG portion.

In pcDNA 3.4 (Invitrogen, A14697; plasmid 1), DNA segment 1 with the nucleotide sequence encoding the heavy chain of the IgG antibody of anti-HER2/anti-4-1BB bispecific antibody was inserted, and in pcDNA 3.4 (Invitrogen, A14697; plasmid 2), DNA segment 2 with the nucleotide sequence encoding the light chain of the IgG antibody of anti-HER2/anti-4-1BB bispecific antibody was inserted. Then, using DNA segment 4 encoding a 15 amino acid long peptide linker consisting of (GGGGS)3 (SEQ ID NO: 85) or DNA segment 5 encoding an 18 amino acid long peptide linker consisting of (GS)9 (SEQ ID NO: 86), a vector for expression of the bispecific antibody was prepared by fusing DNA segment 3 encoding scFv to a portion of DNA segment 1 corresponding to the c-terminus of the Fc region of the IgG antibody inserted into plasmid 1. Furthermore, to stabilize the scFv, a further modification was applied to generate a disulfide bridge fusing VL103-VH44 (VL103: VL with a G→C mutation at position 103; VH44: VH with a G→C mutation at position 44) at the C-terminus of the light chain and the C-terminus of the heavy chain, respectively, as described in Example 1.2.

The sequences of the heavy chain, light chain, scFv, and DNA fragments used to make some exemplary bispecific antibodies are exemplified in Tables 21 through 29. One or more point mutations in the amino acid sequence may be incorporated into the antibodies presented below for improved stability and potency, reduced immunogenicity, etc.

TABLE 21

| HER2 (NA)x1A10 bispecific | | | |
|---|---|---|---|
| antibody-1 | | | Amino acid sequence (N′→C′) |
| Heavy | [symbol] Heavy chain of | | EVQLVESGGGLVQPGGSLRLSCAASGFNIKDTYIHWV |
| component | anti-HER2 antibody | | RQAPGKGLEWVARIYPTNGYTRYADSVKGRFTISADT |
| | | | SKNTAYLQMNSLRAEDTAVYYCSRWGGDGFYAMDY |
| | | | WGQGTLVTVSSASTKGPSVFPLAPSSKSTSGGTAAL |
| | | | GCLVKDYFPEPVTVSWNSGALTSGVHTFPAVLQSSG |
| | | | LYSLSSVVTVPSSSLGTQTYICNVNHKPSNTKVDKKV |
| | | | EPKSCDKTHTCPPCPAPELLGGPSVFLFPPKPKDTLM |
| | | | ISRTPEVTCVVVDVSHEDPEVKFNWYVDGVEVHNAK |
| | | | TKPREEQYASTYRVVSVLTVLHQDWLNGKEYKCKVS |
| | | | NKALPAPIEKTISKAKGQPREPQVYTLPPSREEMTKN |
| | | | QVSLTCLVKGFYPSDIAVEWESNGQPENNYKTTPPVL |
| | | | DSDGSFFLYSKLTVDKSRWQQGNVFSCSVMHEALH |
| | | | NHYTQKSLSLSPGK (SEQ ID NO: 74) |
| | [symbol] Linker | | GGGGSGGGGSGGGGS (SEQ ID NO: 85) |
| | [symbol] scFv | VL | QSVLTQPPSASGTPGRRVTISCSGSSSNIGNNYVTW |
| | of anti-4- | | YQQLPGTAPKLLIYADSHRPSGVPDRFSGSKSGTSAS |
| | 1BB | | LAISGLRSEDEADYYCATWDYSLSGYVFGCGTKLTVL |
| | antibody | | (SEQ ID NO: 33) |
| | | Linker | GGGGSGGGGSGGGGSGGGGS (SEQ ID NO: 87) |
| | | VH | EVQLLESGGGLVQPGGSLRLSCAASGFTFSSYDMSW |
| | | | VRQAPGKCLEWVSWISYSGGSIYYADSVKGRFTISRD |
| | | | NSKNTLYLQMNSLRAEDTAVYYCARDGQRNSMREF |
| | | | DYWGQGTLVTVSS (SEQ ID NO: 24) |

| | | |
|---|---|---|
| | Heavy component | EVQLVESGGGLVQPGGSLRLSCAASGFNIKDTYIHWV |
| | ( [symbol] + [symbol] + [symbol] ) | RQAPGKGLEWVARIYPTNGYTRYADSVKGRFTISADT |
| | | SKNTAYLQMNSLRAEDTAVYYCSRWGGDGFYAMDY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKKV |
| | | EPKSCDKTHTCPPCPAPELLGGPSVFLFPPKPKDTLM |
| | | ISRTPEVTCVVVDVSHEDPEVKFNWYVDGVEVHNAK |
| | | TKPREEQYASTYRVVSVLTVLHQDWLNGKEYKCKVS |
| | | NKALPAPIEKTISKAKGQPREPQVYTLPPSREEMTKN |
| | | QVSLTCLVKGFYPSDIAVEWESNGQPENNYKTTPPVL |
| | | DSDGSFFLYSKLTVDKSRWQQGNVFSCSVMHEALH |
| | | NHYTQKSLSLSPGKGGGGSGGGGSGGGGSQSVLTQ |
| | | PPSASGTPGRRVTISCSGSSSNIGNNYVTWYQQLPG |
| | | TAPKLLIYADSHRPSGVPDRFSGSKSGTSASLAISGLR |
| | | SEDEADYYCATWDYSLSGYVFGCGTKLTVLGGGGS |
| | | GGGGSGGGGSGGGGSEVQLLESGGGLVQPGGSLR |
| | | LSCAASGFTFSSYDMSWVRQAPGKCLEWVSWISYS |
| | | GGSIYYADSVKGRFTISRDNSKNTLYLQMNSLRAEDT |
| | | AVYYCARDGQRNSMREFDYWGQGTLVTVSS (SEQ |
| | | ID NO: 76) |
| Light | Light chain of anti- | DIQMTQSPSSLSASVGDRVTITCRASQDVNTAVAWY |
| component | HER2 antibody | QQKPGKAPKLLIYSASFLYSGVPSRFSGSRSGTDFTL |
| | | TISSLQPEDFATYYCQQHYTTPPTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC (SEQ |
| | | ID NO: 75) |

TABLE 22

| HER2 (NA)x1A10 bispecific | | | |
|---|---|---|---|
| antibody-2 | | | Amino acid sequence (N′→C′) |
| Heavy | [symbol] Heavy chain of | | EVQLVESGGGLVQPGGSLRLSCAASGFNIKDTYIHWVRQA |
| component | anti-HER2 antibody | | PGKGLEWVARIYPTNGYTRYADSVKGRFTISADTSKNTAYL |
| | | | QMNSLRAEDTAVYYCSRWGGDGFYAMDYWGQGTLVTVS |
| | | | SASTKGPSVFPLAPSSKSTSGGTAALGCLVKDYFPEPVTVS |
| | | | WNSGALTSGVHTFPAVLQSSGLYSLSSVVTVPSSSLGTQT |
| | | | YICNVNHKPSNTKVDKKVEPKSCDKTHTCPPCPAPELLGG |
| | | | PSVFLFPPKPKDTLMISRTPEVTCVVVDVSHEDPEVKFNWY |
| | | | VDGVEVHNAKTKPREEQYASTYRVVSVLTVLHQDWLNGK |
| | | | EYKCKVSNKALPAPIEKTISKAKGQPREPQVYTLPPSREEM |
| | | | TKNQVSLTCLVKGFYPSDIAVEWESNGQPENNYKTTPPVL |
| | | | DSDGSFFLYSKLTVDKSRWQQGNVFSCSVMHEALHNHYT |
| | | | QKSLSLSPGK (SEQ ID NO: 74) |
| | [symbol] Linker | | GSGSGSGSGSGSGSGSGS (SEQ ID NO: 86) |
| | [symbol] scFv of | VL | QSVLTQPPSASGTPGRRVTISCSGSSSNIGNNYVTWYQQL |
| | | | PGTAPKLLIYADSHRPSGVPDRFSGSKSGTSASLAISGLRS |
| | | | EDEADYYCATWDYSLSGYVFGCGTKLTVL (SEQ ID NO: 33) |
| | anti-4-1BB | Linker | GGGGSGGGGSGGGGSGGGGS (SEQ ID NO: 87) |
| | antibody | VH | EVQLLESGGGLVQPGGSLRLSCAASGFTFSSYDMSWVRQ |
| | | | APGKCLEWVSWISYSGGSIYYADSVKGRFTISRDNSKNTLY |
| | | | LQMNSLRAEDTAVYYCARDGQRNSMREFDYWGQGTLVT |
| | | | VSS (SEQ ID NO: 24) |

| | | |
|---|---|---|
| | Heavy component | EVQLVESGGGLVQPGGSLRLSCAASGFNIKDTYIHWVRQA |
| | ( [symbol] + [symbol] + [symbol] ) | PGKGLEWVARIYPTNGYTRYADSVKGRFTISADTSKNTAYL |
| | | QMNSLRAEDTAVYYCSRWGGDGFYAMDYWGQGTLVTVS |
| | | SASTKGPSVFPLAPSSKSTSGGTAALGCLVKDYFPEPVTVS |
| | | WNSGALTSGVHTFPAVLQSSGLYSLSSVVTVPSSSLGTQT |
| | | YICNVNHKPSNTKVDKKVEPKSCDKTHTCPPCPAPELLGG |
| | | PSVFLFPPKPKDTLMISRTPEVTCVVVDVSHEDPEVKFNWY |
| | | VDGVEVHNAKTKPREEQYASTYRVVSVLTVLHQDWLNGK |
| | | EYKCKVSNKALPAPIEKTISKAKGQPREPQVYTLPPSREEM |
| | | TKNQVSLTCLVKGFYPSDIAVEWESNGQPENNYKTTPPVL |
| | | DSDGSFFLYSKLTVDKSRWQQGNVFSCSVMHEALHNHYT |
| | | QKSLSLSPGKGSGSGSGSGSGSGSGSGSQSVLTQPPSAS |
| | | GTPGRRVTISCSGSSSNIGNNYVTWYQQLPGTAPKLLIYAD |
| | | SHRPSGVPDRFSGSKSGTSASLAISGLRSEDEADYYCATW |
| | | DYSLSGYVFGCGTKLTVLGGGGSGGGGSGGGGSGGGGS |
| | | EVQLLESGGGLVQPGGSLRLSCAASGFTFSSYDMSWVRQ |
| | | APGKCLEWVSWISYSGGSIYYADSVKGRFTISRDNSKNTLY |
| | | LQMNSLRAEDTAVYYCARDGQRNSMREFDYWGQGTLVT |
| | | VSS (SEQ ID NO: 77) |
| Light | Light chain of anti- | DIQMTQSPSSLSASVGDRVTITCRASQDVNTAVAWYQQKP |
| component | HER2 antibody | GKAPKLLIYSASFLYSGVPSRFSGSRSGTDFTLTISSLQPED |
| | | FATYYCQQHYTTPPTFGQGTKVEIKRTVAAPSVFIFPPSDE |
| | | QLKSGTASVVCLLNNFYPREAKVQWKVDNALQSGNSQES |
| | | VTEQDSKDSTYSLSSTLTLSKADYEKHKVYACEVTHQGLS |
| | | SPVTKSFNRGEC (SEQ ID NO: 75) |

TABLE 23

| HER2 (NA)x1A10 M4 | | | |
|---|---|---|---|
| bispecific antibody | | | Amino acid sequence (N′→C′) |
| Heavy | [symbol] Heavy chain of | | EVQLVESGGGLVQPGGSLRLSCAASGFNIKDTYIHW |
| component | anti-HER2 antibody | | VRQAPGKGLEWVARIYPTNGYTRYADSVKGRFTISA |
| | | | DTSKNTAYLQMNSLRAEDTAVYYCSRWGGDGFYAM |
| | | | DYWGQGTLVTVSSASTKGPSVFPLAPSSKSTSGGTA |
| | | | ALGCLVKDYFPEPVTVSWNSGALTSGVHTFPAVLQS |
| | | | SGLYSLSSVVTVPSSSLGTQTYICNVNHKPSNTKVDK |
| | | | KVEPKSCDKTHTCPPCPAPELLGGPSVFLFPPKPKDT |
| | | | LMISRTPEVTCVVVDVSHEDPEVKFNWYVDGVEVHN |
| | | | AKTKPREEQYASTYRVVSVLTVLHQDWLNGKEYKCK |
| | | | VSNKALPAPIEKTISKAKGQPREPQVYTLPPSREEMT |
| | | | KNQVSLTCLVKGFYPSDIAVEWESNGQPENNYKTTP |
| | | | PVLDSDGSFFLYSKLTVDKSRWQQGNVFSCSVMHE |
| | | | ALHNHYTQKSLSLSPGK (SEQ ID NO: 74) |
| | [symbol] Linker | | GSGSGSGSGSGSGSGSGS (SEQ ID NO: 86) |
| | [symbol] scFv | VL | QSVLTQPPSASGTPGRRVTISCSGSSSNIGNNYVTW |
| | of anti-4- | | YQQLPGTAPKLLIYADSHRPSGVPDRFSGSKSGTSA |
| | 1BB | | SLAISGLRSEDEADYYCATWDYSLSGYVFGCGTKLT |
| | antibody | | VL (SEQ ID NO: 33) |
| | | Linker | GGGGSGGGGSGGGGSGGGGS (SEQ ID NO: 87) |
| | | VH | EVQLLESGGGLVQPGGSLRLSCAASGFTFSSYDMS |
| | | | WVRQAPGKCLEWVSWISYSGGSIYYADSVKGRFTIS |
| | | | RDNSKNTLYLQMNSLRAEDTAVYYCARDAQRNSMR |
| | | | EFDYWGQGTLVTVSS (SEQ ID NO: 25) |

| | | |
|---|---|---|
| | Heavy component | EVQLVESGGGLVQPGGSLRLSCAASGFNIKDTYIHW |
| | ( [symbol] + [symbol] + [symbol] ) | VRQAPGKGLEWVARIYPTNGYTRYADSVKGRFTISA |
| | | DTSKNTAYLQMNSLRAEDTAVYYCSRWGGDGFYAM |
| | | DYWGQGTLVTVSSASTKGPSVFPLAPSSKSTSGGTA |
| | | ALGCLVKDYFPEPVTVSWNSGALTSGVHTFPAVLQS |
| | | SGLYSLSSVVTVPSSSLGTQTYICNVNHKPSNTKVDK |
| | | KVEPKSCDKTHTCPPCPAPELLGGPSVFLFPPKPKDT |
| | | LMISRTPEVTCVVVDVSHEDPEVKFNWYVDGVEVHN |
| | | AKTKPREEQYASTYRVVSVLTVLHQDWLNGKEYKCK |
| | | VSNKALPAPIEKTISKAKGQPREPQVYTLPPSREEMT |
| | | KNQVSLTCLVKGFYPSDIAVEWESNGQPENNYKTTP |
| | | PVLDSDGSFFLYSKLTVDKSRWQQGNVFSCSVMHE |
| | | ALHNHYTQKSLSLSPGKGSGSGSGSGSGSGSGSGS |
| | | QSVLTQPPSASGTPGRRVTISCSGSSSNIGNNYVTW |
| | | YQQLPGTAPKLLIYADSHRPSGVPDRFSGSKSGTSA |
| | | SLAISGLRSEDEADYYCATWDYSLSGYVFGCGTKLT |
| | | VLGGGGSGGGGSGGGGGGGGSEVQLLESGGGLV |
| | | QPGGSLRLSCAASGFTFSSYDMSWVRQAPGKCLEW |
| | | VSWISYSGGSIYYADSVKGRFTISRDNSKNTLYLQMN |
| | | SLRAEDTAVYYCARDAQRNSMREFDYWGQGTLVTV |
| | | SS (SEQ ID NO: 78) |
| Light | Light chain of anti- | DIQMTQSPSSLSASVGDRVTITCRASQDVNTAVAWY |
| component | HER2 antibody | QQKPGKAPKLLIYSASFLYSGVPSRFSGSRSGTDFTL |
| | | TISSLOPEDFATYYCQQHYTTPPTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC (SEQ |
| | | ID NO: 75) |

TABLE 24

| HER2 (NA)x1A10 M12 | | | |
|---|---|---|---|
| bispecific antibody | | | Amino acid sequence (N′→C′) |
| Heavy | [symbol] Heavy chain of | | EVQLVESGGGLVQPGGSLRLSCAASGFNIKDTYIHW |
| component | anti-HER2 antibody | | VRQAPGKGLEWVARIYPTNGYTRYADSVKGRFTISA |
| | | | DTSKNTAYLQMNSLRAEDTAVYYCSRWGGDGFYAM |
| | | | DYWGQGTLVTVSSASTKGPSVFPLAPSSKSTSGGTA |
| | | | ALGCLVKDYFPEPVTVSWNSGALTSGVHTFPAVLQS |
| | | | SGLYSLSSVVTVPSSSLGTQTYICNVNHKPSNTKVDK |
| | | | KVEPKSCDKTHTCPPCPAPELLGGPSVFLFPPKPKDT |
| | | | LMISRTPEVTCVVVDVSHEDPEVKFNWYVDGVEVHN |
| | | | AKTKPREEQYASTYRVVSVLTVLHQDWLNGKEYKCK |
| | | | VSNKALPAPIEKTISKAKGQPREPQVYTLPPSREEMT |
| | | | KNQVSLTCLVKGFYPSDIAVEWESNGQPENNYKTTP |
| | | | PVLDSDGSFFLYSKLTVDKSRWQQGNVFSCSVMHE |
| | | | ALHNHYTQKSLSLSPGK (SEQ ID NO: 74) |
| | [symbol] Linker | | GSGSGSGSGSGSGSGSGS (SEQ ID NO: 86) |
| | [symbol] scFv | VL | QSVLTQPPSASGTPGQRVTISCSGSSSNIGNNYVTW |
| | of anti-4- | | YQQLPGTAPKLLIYADSHRPSGVPDRFSGSKSGTSA |
| | 1BB | | SLAISGLRSEDEADYYCATWDYSLSGYVFGCGTKLT |
| | antibody | | VL (SEQ ID NO: 34) |
| | | Linker | GGGGSGGGGSGGGGSGGGGS (SEQ ID NO: 87) |
| | | VH | EVQLLESGGGLVQPGGSLRLSCAASGFTFSSYDMS |
| | | | WVRQAPGKCLEWVSWISYSGGSIYYADSVKGRFTIS |
| | | | RDNSKNTLYLQMNSLRAEDTAVYYCARDAQRNSMR |
| | | | EFDYWGQGTLVTVSS (SEQ ID NO: 25) |

| | | |
|---|---|---|
| | Heavy component | EVQLVESGGGLVQPGGSLRLSCAASGFNIKDTYIHW |
| | ( [symbol] + [symbol] + [symbol] ) | VRQAPGKGLEWVARIYPTNGYTRYADSVKGRFTISA |
| | | DTSKNTAYLQMNSLRAEDTAVYYCSRWGGDGFYAM |
| | | DYWGQGTLVTVSSASTKGPSVFPLAPSSKSTSGGTA |
| | | ALGCLVKDYFPEPVTVSWNSGALTSGVHTFPAVLQS |
| | | SGLYSLSSVVTVPSSSLGTQTYICNVNHKPSNTKVDK |
| | | KVEPKSCDKTHTCPPCPAPELLGGPSVFLFPPKPKDT |
| | | LMISRTPEVTCVVVDVSHEDPEVKFNWYVDGVEVHN |
| | | AKTKPREEQYASTYRVVSVLTVLHQDWLNGKEYKCK |
| | | VSNKALPAPIEKTISKAKGQPREPQVYTLPPSREEMT |
| | | KNQVSLTCLVKGFYPSDIAVEWESNGQPENNYKTTP |
| | | PVLDSDGSFFLYSKLTVDKSRWQQGNVFSCSVMHE |
| | | ALHNHYTQKSLSLSPGKGSGSGSGSGSGSGSGSGS |
| | | QSVLTQPPSASGTPGQRVTISCSGSSSNIGNNYVTW |
| | | YQQLPGTAPKLLIYADSHRPSGVPDRFSGSKSGTSA |
| | | SLAISGLRSEDEADYYCATWDYSLSGYVFGCGTKLT |
| | | VLGGGGSGGGGSGGGGSGGGGSEVQLLESGGGLV |
| | | QPGGSLRLSCAASGFTFSSYDMSWVRQAPGKCLEW |
| | | VSWISYSGGSIYYADSVKGRFTISRDNSKNTLYLQMN |
| | | SLRAEDTAVYYCARDAQRNSMREFDYWGQGTLVTV |
| | | SS (SEQ ID NO: 79) |
| Light | Light chain of anti- | DIQMTQSPSSLSASVGDRVTITCRASQDVNTAVAWY |
| component | HER2 antibody | QQKPGKAPKLLIYSASFLYSGVPSRFSGSRSGTDFTL |
| | | TISSLQPEDFATYYCQQHYTTPPTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC (SEQ |
| | | ID NO: 75) |

TABLE 25

| HER2 (NA)x1A 12 bispecific | | | |
|---|---|---|---|
| antibody-1 | | | Amino acid sequence (N′→C′) |
| Heavy | [symbol] Heavy chain of | | EVQLVESGGGLVQPGGSLRLSCAASGFNIKDTYIHW |
| component | anti-HER2 antibody | | VRQAPGKGLEWVARIYPTNGYTRYADSVKGRFTISA |
| | | | DTSKNTAYLQMNSLRAEDTAVYYCSRWGGDGFYAM |
| | | | DYWGQGTLVTVSSASTKGPSVFPLAPSSKSTSGGTA |
| | | | ALGCLVKDYFPEPVTVSWNSGALTSGVHTFPAVLQS |
| | | | SGLYSLSSVVTVPSSSLGTQTYICNVNHKPSNTKVDK |
| | | | KVEPKSCDKTHTCPPCPAPELLGGPSVFLFPPKPKDT |
| | | | LMISRTPEVTCVVVDVSHEDPEVKFNWYVDGVEVHN |
| | | | AKTKPREEQYASTYRVVSVLTVLHQDWLNGKEYKCK |
| | | | VSNKALPAPIEKTISKAKGQPREPQVYTLPPSREEMT |
| | | | KNQVSLTCLVKGFYPSDIAVEWESNGQPENNYKTTP |
| | | | PVLDSDGSFFLYSKLTVDKSRWQQGNVFSCSVMHE |
| | | | ALHNHYTQKSLSLSPGK (SEQ ID NO: 74) |
| | [symbol] Linker | | GGGGSGGGGSGGGGS (SEQ ID NO: 85) |
| | [symbol] scFv | VL | QSVLTQPPSASGTPGRRVTISCSGSSSNIGNNYVTW |
| | of anti-4- | | YQQLPGTAPKLLIYADSHRPSGVPDRFSGSKSGTSA |
| | 1BB | | SLAISGLRSEDEADYYCATWDYSLSGYVFGCGTKLT |
| | antibody | | VL (SEQ ID NO: 33) |
| | | Linker | GGGGSGGGGSGGGGSGGGGS (SEQ ID NO: 87) |
| | | VH | EVQLLESGGGLVQPGGSLRLSCAASGFTFSGYDMS |
| | | | WVRQAPGKCLEWVSVIYPDDGNTYYADSVKGRFTIS |
| | | | RDNSKNTLYLQMNSLRAEDAAVYYCAKHGGQKPTTK |
| | | | SSSAYGMDGWGQGTLVTVSS (SEQ ID NO: 27) |

| | | |
|---|---|---|
| | Heavy component | EVQLVESGGGLVQPGGSLRLSCAASGFNIKDTYIHW |
| | ( [symbol] + [symbol] + [symbol] ) | VRQAPGKGLEWVARIYPTNGYTRYADSVKGRFTISA |
| | | DTSKNTAYLQMNSLRAEDTAVYYCSRWGGDGFYAM |
| | | DYWGQGTLVTVSSASTKGPSVFPLAPSSKSTSGGTA |
| | | ALGCLVKDYFPEPVTVSWNSGALTSGVHTFPAVLQS |
| | | SGLYSLSSVVTVPSSSLGTQTYICNVNHKPSNTKVDK |
| | | KVEPKSCDKTHTCPPCPAPELLGGPSVFLFPPKPKDT |
| | | LMISRTPEVTCVVVDVSHEDPEVKFNWYVDGVEVHN |
| | | AKTKPREEQYASTYRVVSVLTVLHQDWLNGKEYKCK |
| | | VSNKALPAPIEKTISKAKGQPREPQVYTLPPSREEMT |
| | | KNQVSLTCLVKGFYPSDIAVEWESNGQPENNYKTTP |
| | | PVLDSDGSFFLYSKLTVDKSRWQQGNVFSCSVMHE |
| | | ALHNHYTQKSLSLSPGKGGGGSGGGGSGGGGSQS |
| | | VLTQPPSASGTPGRRVTISCSGSSSNIGNNYVTWYQ |
| | | QLPGTAPKLLIYADSHRPSGVPDRFSGSKSGTSASLA |
| | | ISGLRSEDEADYYCATWDYSLSGYVFGCGTKLTVLG |
| | | GGGSGGGGSGGGGSGGGGSEVQLLESGGGLVQP |
| | | GGSLRLSCAASGFTFSGYDMSWVRQAPGKCLEWVS |
| | | VIYPDDGNTYYADSVKGRFTISRDNSKNTLYLQMNSL |
| | | RAEDAAVYYCAKHGGQKPTTKSSSAYGMDGWGQG |
| | | TLVTVSS (SEQ ID NO: 80) |
| Light | Light chain of anti- | DIQMTQSPSSLSASVGDRVTITCRASQDVNTAVAWY |
| component | HER2 antibody | QQKPGKAPKLLIYSASFLYSGVPSRFSGSRSGTDFTL |
| | | TISSLQPEDFATYYCQQHYTTPPTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC (SEQ |
| | | ID NO: 75) |

TABLE 26

| HER2 (NA)x1A12 bispecific | | | |
|---|---|---|---|
| antibody-2 | | | Amino acid sequence (N′→C′) |
| Heavy | [symbol] Heavy chain of | | EVQLVESGGGLVQPGGSLRLSCAASGFNIKDTYIHW |
| component | anti-HER2 antibody | | VRQAPGKGLEWVARIYPTNGYTRYADSVKGRFTISA |
| | | | DTSKNTAYLQMNSLRAEDTAVYYCSRWGGDGFYAM |
| | | | DYWGQGTLVTVSSASTKGPSVFPLAPSSKSTSGGTA |
| | | | ALGCLVKDYFPEPVTVSWNSGALTSGVHTFPAVLQS |
| | | | SGLYSLSSVVTVPSSSLGTQTYICNVNHKPSNTKVDK |
| | | | KVEPKSCDKTHTCPPCPAPELLGGPSVFLFPPKPKDT |
| | | | LMISRTPEVTCVVVDVSHEDPEVKFNWYVDGVEVHN |
| | | | AKTKPREEQYASTYRVVSVLTVLHQDWLNGKEYKCK |
| | | | VSNKALPAPIEKTISKAKGQPREPQVYTLPPSREEMT |
| | | | KNQVSLTCLVKGFYPSDIAVEWESNGQPENNYKTTP |
| | | | PVLDSDGSFFLYSKLTVDKSRWQQGNVFSCSVMHE |
| | | | ALHNHYTQKSLSLSPGK (SEQ ID NO: 74) |
| | [symbol] Linker | | GSGSGSGSGSGSGSGSGS (SEQ ID NO: 86) |
| | [symbol] scFv | VL | QSVLTQPPSASGTPGRRVTISCSGSSSNIGNNYVTW |
| | of anti-4- | | YQQLPGTAPKLLIYADSHRPSGVPDRFSGSKSGTSA |
| | 1BB | | SLAISGLRSEDEADYYCATWDYSLSGYVFGCGTKLT |
| | antibody | | VL (SEQ ID NO: 33) |
| | | Linker | GGGGSGGGGSGGGGSGGGGS (SEQ ID NO: 87) |
| | | VH | EVQLLESGGGLVQPGGSLRLSCAASGFTFSGYDMS |
| | | | WVRQAPGKCLEWVSVIYPDDGNTYYADSVKGRFTIS |
| | | | RDNSKNTLYLQMNSLRAEDAAVYYCAKHGGQKPTTK |
| | | | SSSAYGMDGWGQGTLVTVSS (SEQ ID NO: 27) |

| | | |
|---|---|---|
| | Heavy component | EVQLVESGGGLVQPGGSLRLSCAASGFNIKDTYIHW |
| | ( [symbol] + [symbol] + [symbol] ) | VRQAPGKGLEWVARIYPTNGYTRYADSVKGRFTISA |
| | | DTSKNTAYLQMNSLRAEDTAVYYCSRWGGDGFYAM |
| | | DYWGQGTLVTVSSASTKGPSVFPLAPSSKSTSGGTA |
| | | ALGCLVKDYFPEPVTVSWNSGALTSGVHTFPAVLQS |
| | | SGLYSLSSVVTVPSSSLGTQTYICNVNHKPSNTKVDK |
| | | KVEPKSCDKTHTCPPCPAPELLGGPSVFLFPPKPKDT |
| | | LMISRTPEVTCVVVDVSHEDPEVKFNWYVDGVEVHN |
| | | AKTKPREEQYASTYRVVSVLTVLHQDWLNGKEYKCK |
| | | VSNKALPAPIEKTISKAKGQPREPQVYTLPPSREEMT |
| | | KNQVSLTCLVKGFYPSDIAVEWESNGQPENNYKTTP |
| | | PVLDSDGSFFLYSKLTVDKSRWQQGNVFSCSVMHE |
| | | ALHNHYTQKSLSLSPGKGSGSGSGSGSGSGSGSGS |
| | | QSVLTQPPSASGTPGRRVTISCSGSSSNIGNNYVTW |
| | | YQQLPGTAPKLLIYADSHRPSGVPDRFSGSKSGTSA |
| | | SLAISGLRSEDEADYYCATWDYSLSGYVFGCGTKLT |
| | | VLGGGGSGGGGSGGGGSGGGGSEVQLLESGGGLV |
| | | QPGGSLRLSCAASGFTFSGYDMSWVRQAPGKCLEW |
| | | VSVIYPDDGNTYYADSVKGRFTISRDNSKNTLYLQMN |
| | | SLRAEDAAVYYCAKHGGQKPTTKSSSAYGMDGWGQ |
| | | GTLVTVSS (SEQ ID NO: 81) |
| Light | Light chain of a | DIQMTQSPSSLSASVGDRVTITCRASQDVNTAVAWY |
| component | anti-HER2 antibody | QQKPGKAPKLLIYSASFLYSGVPSRFSGSRSGTDFTL |
| | | TISSLQPEDFATYYCQQHYTTPPTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC (SEQ |
| | | ID NO: 75) |

TABLE 27

| HER2 (NA)x1A12 M1 | | | |
|---|---|---|---|
| bispecific antibody | | | Amino acid sequence (N′→C′) |
| Heavy | [symbol] Heavy chain of | | EVQLVESGGGLVQPGGSLRLSCAASGFNIKDTYIHW |
| component | anti-HER2 antibody | | VRQAPGKGLEWVARIYPTNGYTRYADSVKGRFTISA |
| | | | DTSKNTAYLQMNSLRAEDTAVYYCSRWGGDGFYAM |
| | | | DYWGQGTLVTVSSASTKGPSVFPLAPSSKSTSGGTA |
| | | | ALGCLVKDYFPEPVTVSWNSGALTSGVHTFPAVLQS |
| | | | SGLYSLSSVVTVPSSSLGTQTYICNVNHKPSNTKVDK |
| | | | KVEPKSCDKTHTCPPCPAPELLGGPSVFLFPPKPKDT |
| | | | LMISRTPEVTCVVVDVSHEDPEVKFNWYVDGVEVHN |
| | | | AKTKPREEQYASTYRVVSVLTVLHQDWLNGKEYKCK |
| | | | VSNKALPAPIEKTISKAKGQPREPQVYTLPPSREEMT |
| | | | KNQVSLTCLVKGFYPSDIAVEWESNGQPENNYKTTP |
| | | | PVLDSDGSFFLYSKLTVDKSRWQQGNVFSCSVMHE |
| | | | ALHNHYTQKSLSLSPGK (SEQ ID NO: 74) |
| | [symbol] Linker | | GSGSGSGSGSGSGSGSGS (SEQ ID NO: 86) |
| | [symbol] scFv | VL | QSVLTQPPSASGTPGQRVTISCSGSSSNIGNNYVTW |
| | of anti-4- | | YQQLPGTAPKLLIYADSHRPSGVPDRFSGSKSGTSA |
| | 1BB | | SLAISGLRSEDEADYYCATWDYSLSGYVFGCGTKLT |
| | antibody | | VL (SEQ ID NO: 34) |
| | | Linker | GGGGSGGGGSGGGGSGGGGS (SEQ ID NO: 87) |
| | | VH | EVQLLESGGGLVQPGGSLRLSCAASGFTFSGYDMS |
| | | | WVRQAPGKCLEWVSVIYPDDGNTYYADSVKGRFTIS |
| | | | RDNSKNTLYLQMNSLRAEDTAVYYCAKHGGQKPTTK |
| | | | SSSAYGMDGWGQGTLVTVSS (SEQ ID NO: 28) |

| | | |
|---|---|---|
| | Heavy component | EVQLVESGGGLVQPGGSLRLSCAASGFNIKDTYIHW |
| | ( [symbol] + [symbol] + [symbol] ) | VRQAPGKGLEWVARIYPTNGYTRYADSVKGRFTISA |
| | | DTSKNTAYLQMNSLRAEDTAVYYCSRWGGDGFYAM |
| | | DYWGQGTLVTVSSASTKGPSVFPLAPSSKSTSGGTA |
| | | ALGCLVKDYFPEPVTVSWNSGALTSGVHTFPAVLQS |
| | | SGLYSLSSVVTVPSSSLGTQTYICNVNHKPSNTKVDK |
| | | KVEPKSCDKTHTCPPCPAPELLGGPSVFLFPPKPKDT |
| | | LMISRTPEVTCVVVDVSHEDPEVKFNWYVDGVEVHN |
| | | AKTKPREEQYASTYRVVSVLTVLHQDWLNGKEYKCK |
| | | VSNKALPAPIEKTISKAKGQPREPQVYTLPPSREEMT |
| | | KNQVSLTCLVKGFYPSDIAVEWESNGQPENNYKTTP |
| | | PVLDSDGSFFLYSKLTVDKSRWQQGNVFSCSVMHE |
| | | ALHNHYTQKSLSLSPGKGSGSGSGSGSGSGSGSGS |
| | | QSVLTQPPSASGTPGQRVTISCSGSSSNIGNNYVTW |
| | | YQQLPGTAPKLLIYADSHRPSGVPDRFSGSKSGTSA |
| | | SLAISGLRSEDEADYYCATWDYSLSGYVFGCGTKLT |
| | | VLGGGGSGGGGSGGGGSGGGGSEVQLLESGGGLV |
| | | QPGGSLRLSCAASGFTFSGYDMSWVRQAPGKCLEW |
| | | VSVIYPDDGNTYYADSVKGRFTISRDNSKNTLYLQMN |
| | | SLRAEDTAVYYCAKHGGQKPTTKSSSAYGMDGWGQ |
| | | GTLVTVSS (SEQ ID NO: 82) |
| Light | Light chain of anti- | DIQMTQSPSSLSASVGDRVTITCRASQDVNTAVAWY |
| component | HER2 antibody | QQKPGKAPKLLIYSASFLYSGVPSRFSGSRSGTDFTL |
| | | TISSLQPEDFATYYCQQHYTTPPTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC (SEQ |
| | | ID NO: 75) |

TABLE 28

| HER2 (WT)x1A10 M12 | | | |
|---|---|---|---|
| bispecific antibody | | | Amino acid sequence (N′→C′) |
| Heavy | [symbol] Heavy chain of | | EVQLVESGGGLVQPGGSLRLSCAASGFNIKDTYIHW |
| component | anti-HER2 antibody | | VRQAPGKGLEWVARIYPTNGYTRYADSVKGRFTISA |
| | | | DTSKNTAYLQMNSLRAEDTAVYYCSRWGGDGFYAM |
| | | | DYWGQGTLVTVSSASTKGPSVFPLAPSSKSTSGGTA |
| | | | ALGCLVKDYFPEPVTVSWNSGALTSGVHTFPAVLQS |
| | | | SGLYSLSSVVTVPSSSLGTQTYICNVNHKPSNTKVDK |
| | | | KVEPKSCDKTHTCPPCPAPELLGGPSVFLFPPKPKDT |
| | | | LMISRTPEVTCVVVDVSHEDPEVKFNWYVDGVEVHN |
| | | | AKTKPREEQYNSTYRVVSVLTVLHQDWLNGKEYKCK |
| | | | VSNKALPAPIEKTISKAKGQPREPQVYTLPPSREEMT |
| | | | KNQVSLTCLVKGFYPSDIAVEWESNGQPENNYKTTP |
| | | | PVLDSDGSFFLYSKLTVDKSRWQQGNVFSCSVMHE |
| | | | ALHNHYTQKSLSLSPGK (SEQ ID NO: 73) |
| | [symbol] Linker | | GSGSGSGSGSGSGSGSGS (SEQ ID NO: 86) |
| | [symbol] scFv | VL | QSVLTQPPSASGTPGQRVTISCSGSSSNIGNNYVTW |
| | of anti-4- | | YQQLPGTAPKLLIYADSHRPSGVPDRFSGSKSGTSA |
| | 1BB | | SLAISGLRSEDEADYYCATWDYSLSGYVFGCGTKLT |
| | antibody | | VL (SEQ ID NO: 34) |
| | | Linker | GGGGSGGGGSGGGGSGGGGS (SEQ ID NO: 87) |
| | | VH | EVQLLESGGGLVQPGGSLRLSCAASGFTFSSYDMS |
| | | | WVRQAPGKCLEWVSWISYSGGSIYYADSVKGRFTIS |
| | | | RDNSKNTLYLQMNSLRAEDTAVYYCARDAQRNSMR |
| | | | EFDYWGQGTLVTVSS (SEQ ID NO: 25) |

| | | |
|---|---|---|
| | Heavy component | EVQLVESGGGLVQPGGSLRLSCAASGFNIKDTYIHW |
| | ( [symbol] + [symbol] + [symbol] ) | VRQAPGKGLEWVARIYPTNGYTRYADSVKGRFTISA |
| | | DTSKNTAYLQMNSLRAEDTAVYYCSRWGGDGFYAM |
| | | DYWGQGTLVTVSSASTKGPSVFPLAPSSKSTSGGTA |
| | | ALGCLVKDYFPEPVTVSWNSGALTSGVHTFPAVLQS |
| | | SGLYSLSSVVTVPSSSLGTQTYICNVNHKPSNTKVDK |
| | | KVEPKSCDKTHTCPPCPAPELLGGPSVFLFPPKPKDT |
| | | LMISRTPEVTCVVVDVSHEDPEVKFNWYVDGVEVHN |
| | | AKTKPREEQYNSTYRVVSVLTVLHQDWLNGKEYKCK |
| | | VSNKALPAPIEKTISKAKGQPREPQVYTLPPSREEMT |
| | | KNQVSLTCLVKGFYPSDIAVEWESNGQPENNYKTTP |
| | | PVLDSDGSFFLYSKLTVDKSRWQQGNVFSCSVMHE |
| | | ALHNHYTQKSLSLSPGKGSGSGSGSGSGSGSGSGS |
| | | QSVLTQPPSASGTPGQRVTISCSGSSSNIGNNYVTW |
| | | YQQLPGTAPKLLIYADSHRPSGVPDRFSGSKSGTSA |
| | | SLAISGLRSEDEADYYCATWDYSLSGYVFGCGTKLT |
| | | VLGGGGSGGGGSGGGGSGGGGSEVQLLESGGGLV |
| | | QPGGSLRLSCAASGFTFSSYDMSWVRQAPGKCLEW |
| | | VSWISYSGGSIYYADSVKGRFTISRDNSKNTLYLQMN |
| | | SLRAEDTAVYYCARDAQRNSMREFDYWGQGTLVTV |
| | | SS (SEQ ID NO: 83) |
| Light | Light chain of anti- | DIQMTQSPSSLSASVGDRVTITCRASQDVNTAVAWY |
| component | HER2 antibody | QQKPGKAPKLLIYSASFLYSGVPSRFSGSRSGTDFTL |
| | | TISSLQPEDFATYYCQQHYTTPPTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC (SEQ |
| | | ID NO: 75) |

TABLE 29

| HER2 (WT)x1A12 M1 | | | |
|---|---|---|---|
| bispecific antibody | | | Amino acid sequence (N′→C′) |
| Heavy | [symbol] Heavy chain of | | EVQLVESGGGLVQPGGSLRLSCAASGFNIKDTYIHWV |
| component | anti-HER2 | | RQAPGKGLEWVARIYPTNGYTRYADSVKGRFTISADT |
| | antibody | | SKNTAYLQMNSLRAEDTAVYYCSRWGGDGFYAMDY |
| | | | WGQGTLVTVSSASTKGPSVFPLAPSSKSTSGGTAALG |
| | | | CLVKDYFPEPVTVSWNSGALTSGVHTFPAVLQSSGLY |
| | | | SLSSVVTVPSSSLGTQTYICNVNHKPSNTKVDKKVEPK |
| | | | SCDKTHTCPPCPAPELLGGPSVFLFPPKPKDTLMISRT |
| | | | PEVTCVVVDVSHEDPEVKFNWYVDGVEVHNAKTKPR |
| | | | EEQYNSTYRVVSVLTVLHQDWLNGKEYKCKVSNKALP |
| | | | APIEKTISKAKGQPREPQVYTLPPSREEMTKNQVSLTC |
| | | | LVKGFYPSDIAVEWESNGQPENNYKTTPPVLDSDGSF |
| | | | FLYSKLTVDKSRWQQGNVFSCSVMHEALHNHYTQKS |
| | | | LSLSPGK (SEQ ID NO: 73) |
| | [symbol] Linker | | GSGSGSGSGSGSGSGSGS (SEQ ID NO: 86) |
| | [symbol] scFv | VL | QSVLTQPPSASGTPGQRVTISCSGSSSNIGNNYVTWY |
| | of anti-4- | | QQLPGTAPKLLIYADSHRPSGVPDRFSGSKSGTSASLA |
| | 1BB | | ISGLRSEDEADYYCATWDYSLSGYVFGCGTKLTVL |
| | antibody | | (SEQ ID NO: 34) |
| | | Linker | GGGGSGGGGSGGGGSGGGGS (SEQ ID NO: 87) |
| | | VH | EVQLLESGGGLVQPGGSLRLSCAASGFTFSGYDMSW |
| | | | VRQAPGKCLEWVSVIYPDDGNTYYADSVKGRFTISRD |
| | | | NSKNTLYLQMNSLRAEDTAVYYCAKHGGQKPTTKSSS |
| | | | AYGMDGWGQGTLVTVSS (SEQ ID NO: 28) |

| | | |
|---|---|---|
| | Heavy component | EVQLVESGGGLVQPGGSLRLSCAASGFNIKDTYIHWV |
| | ( [symbol] + [symbol] + [symbol] ) | RQAPGKGLEWVARIYPTNGYTRYADSVKGRFTISADT |
| | | SKNTAYLQMNSLRAEDTAVYYCSRWGGDGFYAMDY |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEPK |
| | | SCDKTHTCPPCPAPELLGGPSVFLFPPKPKDTLMISRT |
| | | PEVTCVVVDVSHEDPEVKFNWYVDGVEVHNAKTKPR |
| | | EEQYNSTYRVVSVLTVLHQDWLNGKEYKCKVSNKALP |
| | | APIEKTISKAKGQPREPQVYTLPPSREEMTKNQVSLTC |
| | | LVKGFYPSDIAVEWESNGQPENNYKTTPPVLDSDGSF |
| | | FLYSKLTVDKSRWQQGNVFSCSVMHEALHNHYTQKS |
| | | LSLSPGKGSGSGSGSGSGSGSGSGSQSVLTQPPSAS |
| | | GTPGQRVTISCSGSSSNIGNNYVTWYQQLPGTAPKLLI |
| | | YADSHRPSGVPDRFSGSKSGTSASLAISGLRSEDEAD |
| | | YYCATWDYSLSGYVFGCGTKLTVLGGGGSGGGGSG |
| | | GGGSGGGGSEVQLLESGGGLVQPGGSLRLSCAASGF |
| | | TFSGYDMSWRQAPGKCLEWVSVIYPDDGNTYYADS |
| | | VKGRFTISRDNSKNTLYLQMNSLRAEDTAVYYCAKHG |
| | | GQKPTTKSSSAYGMDGWGQGTLVTVSS (SEQ ID NO: |
| | | 84) |
| Light | Light chain of | DIQMTQSPSSLSASVGDRVTITCRASQDVNTAVAWYQ |
| component | anti-HER2 | QKPGKAPKLLIYSASFLYSGVPSRFSGSRSGTDFTLTIS |
| | antibody | SLQPEDFATYYCQQHYTTPPTFGQGTKVEIKRTVAAPS |
| | | VFIFPPSDEQLKSGTASVVCLLNNFYPREAKVQWKVD |
| | | NALQSGNSQESVTEQDSKDSTYSLSSTLTLSKADYEK |
| | | HKVYACEVTHQGLSSPVTKSFNRGEC (SEQ ID NO: |
| | | 75) |

### Example 4. Testing the Binding Affinity of Bispecific Antibodies (BsAbs)

### 4.1. Binding to Human HER2

The HER2 binding affinity of the bispecific antibody was performed by ELISA with reference to Example 1.3(1).

Briefly, 96-well microtiter plates (Nunc-Immuno Plates, NUNC) were coated with 100 μl/well of human HER2-His protein (Sino Biological, 10001-H08B) at 1 μg/ml in PBS overnight at 4° C., and then blocked with blocking buffer (200 μl/well of 1% BSA in PBS with bovine serum albumin (Gibco, 30063572)) for 2 hrs at 37° C. Serial dilutions (starting at 0.1 μM) of anti-HER2/anti-4-1BB bispecific antibody prepared in Example 3 and anti-HER2 antibody (HER2(NA)) as a control were added to each well and incubated at 37° C. for 1 hr. The plate was washed with PBS/0.05% Tween20 and incubated with HRP-conjugated Fab antibody (Pierce, 31414) for 1 hr at 37° C. After washing, the plates were developed with Tetramethylbenzidine (TMB, Sigma, T0440) substrate and analyzed spectrophotometrically at OD 450-650 nm.

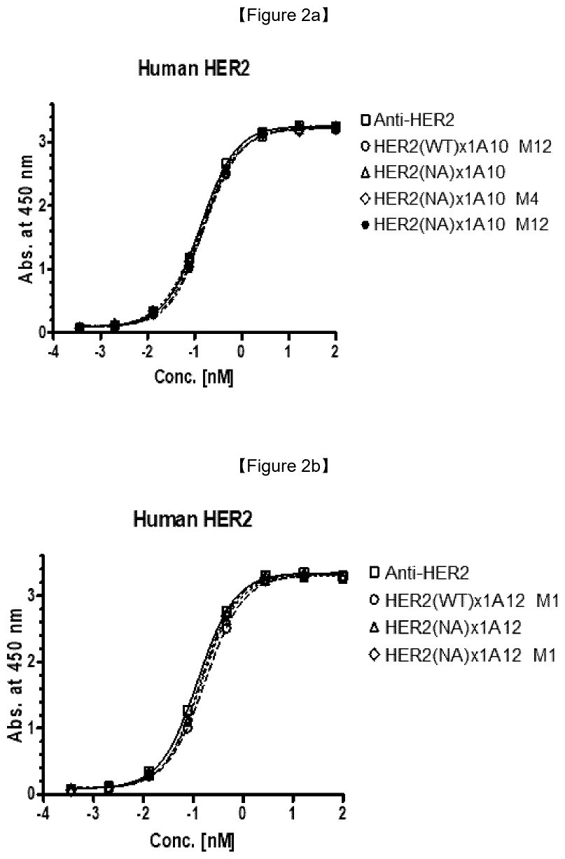

As shown in FIGS. 2a and 2b, the results confirmed that all tested anti-HER2/anti-4-1BB bispecific antibodies were able to bind to human HER2 protein with high affinity similar to that of the control anti-HER2 antibody (NA).

### 4.2. Binding Affinity_ELISA Measurement for Human 4-1BB

The binding affinity of the bispecific antibody to 4-1BB was performed by ELISA with reference to Example 1.3(1). Briefly, 96-well microtiter plates (Nunc-Immuno Plates, NUNC) were coated with human 4-1BB-His protein (Sino Biological, 10041-H08H) at 1 μg/ml in PBS, 100 μl/well overnight at 4° C. The plate was blocked with blocking buffer (200 μl/well of 1% (v/v) bovine serum albumin (BSA) (Gibco, 30063572) in PBS) for 2 hrs at 37° C. Serial dilutions (starting at 0.1 μM) of anti-HER2/anti-4-1BB bispecific antibody prepared in Example 3 and anti-HER2 antibody (HER2(NA)) as a control were added to each well and incubated at 37° C. for 1 hr. The plate was washed with PBS/0.05% Tween20 and incubated with HRP-conjugated Fab antibody (Pierce, 31414) for 1 hr at 37° C. After washing, the plates were developed with TMB substrate and analyzed spectrophotometrically at OD 450-650 nm.

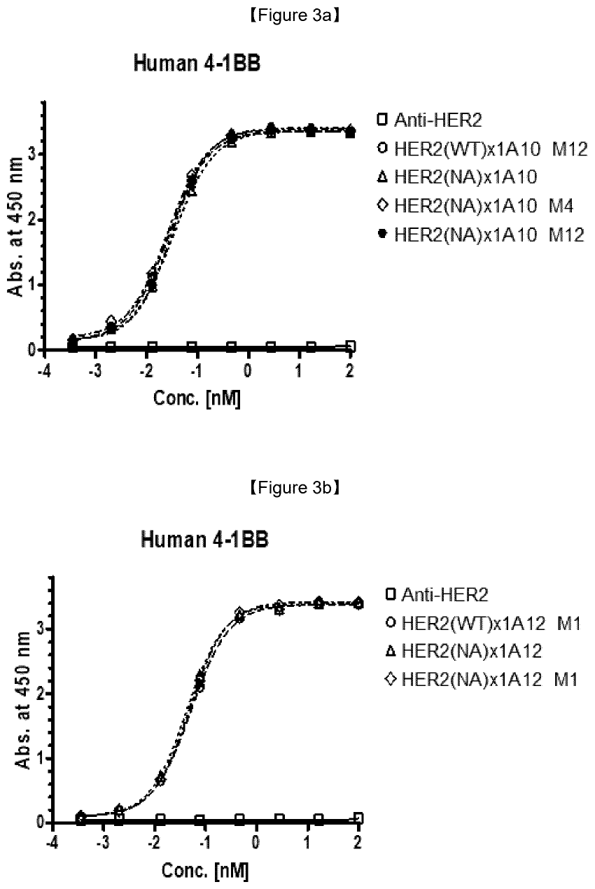

As shown in FIGS. 3a and 3b, all tested anti-HER2/anti-4-1BB bispecific antibodies could bind to human 4-1BB protein with high affinity, while anti-HER2 antibodies did not bind to human 4-1BB protein.

The results of FIGS. 2a, 2b, 3a, and 3b are quantified and summarized in Table 30 below:

TABLE 30

| EC50 (nM) | HER2 | 4-1BB |
|---|---|---|
| HER2(NA) | 0.134 | N/A |
| HER2(WT) × 1A10 M12 | 0.159 | 0.027 |
| HER2(NA) × 1A10 | 0.135 | 0.033 |
| HER2(NA) × 1A10 M4 | 0.148 | 0.025 |
| HER2(NA) × 1A10 M12 | 0.161 | 0.031 |
| HER2(WT) × 1A12 M1 | 0.174 | 0.051 |
| HER2(NA) × 1A12 | 0.148 | 0.043 |
| HER2(NA) × 1A12 M1 | 0.143 | 0.048 |

As shown in Table 30, all tested anti-HER2/anti-4-1BB bispecific antibodies were able to bind to both human HER2 and human 4-1BB proteins with high affinity.

### 4.3. Binding to Various Cell Surface Expressions of Human HER2

Referring to Example 1.3(2) above, the binding affinity of the bispecific antibody to the surface of various cells expressing HER2 was performed by FACS analysis.

The various tumor cell lines listed in Table 31 below were used. After each cell line was isolated and washed with PBS, the cells were counted and set to 2×10⁵ cells/100 μl FACS buffer, then treated with anti-HER2 antibody or anti-HER2/anti-4-1BB bispecific antibody at 10 μg/mL, and reacted for 1 hr at 4° C. After the reaction, cells were washed with FACS buffer and incubated with FITC-labeled constant region (Fc) specific antibody (Goat anti-human IgG FITC conjugate, Fc specific, Sigma, F9512, concentration: 2.0 mg/ml) followed by suspension in 2 μl/2×10⁵ cells/100 μl FACS buffer, and then reacted for 1 hr at 4° C. After the reaction, cells were washed with FACS buffer and analyzed using a FACSCalibur device. Negative controls were treated with FITC-labeled constant region (Fc)-specific antibodies only. To compare the expression degree of HER2 among cancer cell lines, the peak shift result value of the experimental group was divided by the peak shift result value of the negative control group.

The results obtained are shown in Table 31 below.

TABLE 31
MFI (mean fluorescence intensity) ratio

| | | | Anti- | HER2 × | HER2 × |
|---|---|---|---|---|---|
| Cell line | | | HER2 | 1A10 | 1A12 |
| NCl-N87 | Gastric | ATCC, CRL-5822 | 104 | 139 | 145 |
| BT-474 | Breast | ATCC, HTB-20 | 81 | 102 | 90 |
| Calu-3 | Lung | ATCC, HTB-55 | 74 | 82 | 82 |
| HCC1954 | Breast | ATCC, CRL-2338 | 32 | 40 | 42 |
| JIMT1 | Breast | DSMZ, ACC 589 | 26 | 27 | 25 |
| HT29 | Colon | ATCC, HTB-38 | 6.1 | 6.0 | 7.4 |
| MCF-7 | Breast | ATCC, HTB-22 | 5.2 | 4.9 | 5.8 |
| MDA- | Breast | ATCC, HTB-26 | 1.2 | 1.4 | 1.6 |
| MB231 | | | | | |
| H929 | MM | ATCC, CRL-9068 | 0.9 | 1.4 | 1.4 |
| Jurkat | ALL | ATCC, TIB-152 | 1.0 | 1.3 | 1.6 |

(MFI Ratio: MFI of 1^{st} Ab/MFI of 2^{nd} Ab)

As shown in Table 31, all tested anti-HER2/anti-4-1BB bispecific antibodies can bind to cell surface expressing human HER2 proteins.

### 4.4. Binding Affinity for 4-1BB_SPR Measurement

In SPR experiments, anti-HER2/anti-4-1BB bispecific antibodies were captured separately in flow cells 2, 3, and 4, with flow cell 1 kept as a reference. Anti-human Fab antibodies (GE Healthcare, 28958325) were immobilized by amine coupling on a Biocore® Series S sensor chip CM5 (GE Healthcare, BR100530). Recombinant human 4-1BB proteins (ACROBiosystems, 41B-H5227) at concentrations of 400, 200, 100, 50, 25, 12.5, 6.25, 3.13, 1.56, and 0.78 nM, respectively, were flowed across the chip at 30 μl/min for 300 s, followed by a 400 s dissociation step. Regeneration was performed using 10 mM Glycine-HCl (pH 2.0) (GE Healthcare, BR100355).

The results obtained are shown in Table 32 below.

TABLE 32

| Antibody | KD (M) | kon(1/Ms) | kdis(1/s) |
|---|---|---|---|
| HER2(WT) × 1A10 M12 | 2.11E−09 | 2.55E+05 | 5.38E−04 |
| HER2(NA) × 1A10 M12 | 2.35E−09 | 2.69E+05 | 6.33E−04 |
| HER2(WT) × 1A12 M1 | 1.24E−08 | 6.57E+04 | 8.11E−04 |
| HER2(NA) × 1A12 M1 | 1.38E−08 | 6.74E+04 | 9.27E−04 |

As shown in Table 32, the tested anti-HER2/anti-4-1BB bispecific antibodies exhibited high 4-1BB binding affinities.

### Example 5. HER2 Expression Levels in Various Cells

HER2 cell surface expression levels in various cancer cell lines were quantified using the QIFIKIT quantification kit (Dako) according to the manufacturer's recommendations. More specifically, cells were stained with unlabeled anti-HER2 mouse monoclonal antibody (R&D Systems) or purified mouse IgG2b isotype control (R&D Systems) at saturating concentrations. After washing, the stained cells and calibration beads in the kit were simultaneously labeled with the same FITC-conjugated goat anti-mouse IgG secondary antibody in the kit. The labeled cells and calibration beads were analyzed on a flow cytometer. Linear regression was performed using the MFI values of the calibration beads. The antibody binding capacity (ABC) was determined by extrapolating from this regression line, and the specific ABC (sABC) was determined by subtracting the ABC of the isotype control antibody from the ABC of the anti-HER2 antibody. Furthermore, the sABC (HER2 level) of various cell lines obtained above was standardized to the value (100%) of SK-BR-3.

The results obtained are shown in Table 33.

TABLE 33

| Cell lines | | sABC[HER2] | %[SK-BR-3] |
|---|---|---|---|
| NCl-N87 | ATCC, CRL-5822 | 1,624,200 | 225.2 |
| BT-474 | ATCC, HTB-20 | 1,190,606 | 165.1 |
| SK-BR-3 | ATCC, HTB-30 | 721,333 | 100.0 |
| Calu-3 | ATCC, HTB-55 | 706,886 | 98.0 |
| HCC1954 | ATCC, CRL-2338 | 497,805 | 69.0 |
| JIMT-1 | DSMZ, ACC 589 | 93,113 | 12.9 |
| ZR-75-1 | ATCC, CRL-1500 | 25,360 | 3.5 |
| A-431 | ATCC, CRL-1555 | 13,130 | 1.8 |
| MCF7 | ATCC, HTB-22 | 9,916 | 1.4 |
| LoVo | ATCC, CCL-229 | 7,273 | 1.0 |
| MDA-MB-231 | ATCC, HTB-26 | 3,844 | 0.5 |
| BxPC-3 | ATCC, CRL-1687 | 2,013 | 0.3 |

### Example 6. Induction of 4-1BB Signal Activation Dependent on FcγRI Binding of Anti-HER2/Anti-4-1BB Bispecific Antibodies

CHO-K1 cells (GenScript) expressing FcγRI (CD64) were plated (4×10⁴ cells/well) in 96-well assay plates and incubated overnight in a 37° C., 5% CO₂ incubator. On the day of the assay, culture medium was removed from each well and effector cells (NFκB-Luc2/h4-1BB cells; Promega) were plated (5×10⁴ cells/well). Each well was treated with anti-HER2/anti-4-1BB bispecific antibody (HER2(WT)×1A10 M12) or anti-4-1BB antibody (Urelumab, U.S. Pat. No. 7,288,638) and incubated for 6 hrs at 37° C., 5% CO₂ in an incubator. Bio-Glo™ reagent was then added to each well and luminescence was measured using a microplate reader.

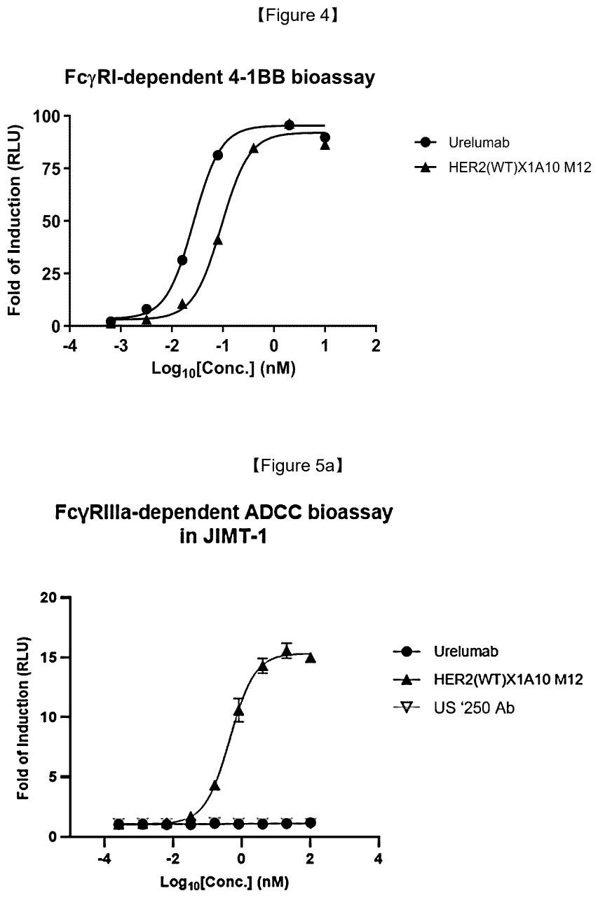

As a result, as shown in FIG. 4, an anti-HER2/anti-4-1BB bispecific antibody (HER2(WT)×1A10 M12) induced strong 4-1BB activation in the presence of FcγRI.

### Example 7. FcγRIIIa-Mediated ADCC Activity of Anti-HER2/Anti-4-1BB Bispecific Antibody in Tumor Cells with HER2-Low Expression

To determine the efficacy of the anti-HER2/anti-4-1BB bispecific antibody in tumor cells with HER2-low expression, the effect on FcγRIIIa-mediated antibody-dependent cellular cytotoxicity (ADCC) was tested. HER2-low expressing cells JIMT-1 and MDA-MB-231 (see Table 33) were plated in 96-well assay plates (1×10⁴ cells/well each) and incubated overnight in a 37° C., 5% CO₂ incubator. On the day of the assay, culture medium was removed from each well, and effector cells (NFκB-Luc2/FcγRIIIa cells; Promega) were plated (5×10⁴ cells/well). Each well was treated with anti-HER2/anti-4-1BB bispecific antibody (HER2(WT)×1A10 M12), US '250 Ab (anti-HER2/anti-4-1BB bispecific antibody, prepared with SEQ ID NOs: 9 and 10 of U.S. Pat. No. 10,865,250), or Urelumab, an anti-4-1BB antibody, respectively, and incubated for 6 hrs at 37° C., 5% CO₂ in an incubator. Bio-Glo™ reagent was then added to each well and luminescence was measured using a microplate reader.

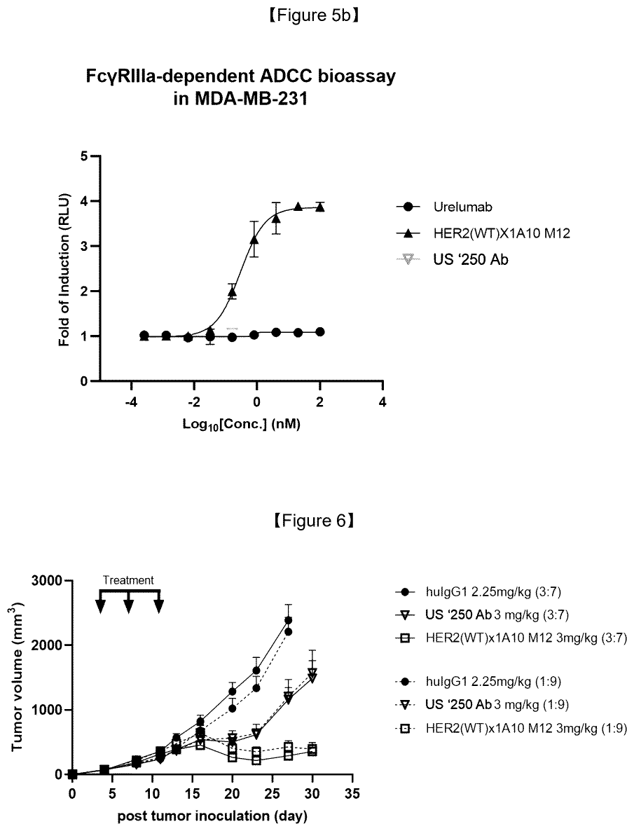

As a result, as shown in FIGS. 5a (JIMT-1 cell line) and 5b (MDA-MB-231 cell line), in both tested HER2-low expressing tumor cells (JIMT-1, MDA-MB-231), the anti-HER2/anti-4-1BB bispecific antibody, US '250 Ab, and the anti-4-1BB antibody, Urelumab, barely induced ADCC activation, whereas the anti-HER2/anti-4-1BB bispecific antibody (HER2(WT)×1A10 M12) remarkably induced ADCC activation.

### Example 8. Antitumor Effect of Anti-HER2/Anti-4-1BB Bispecific Antibody in a Mouse Model with Tumors with HER2-Low Expression

A mouse model was prepared to evaluate the efficacy of an anti-HER2/anti-4-1BB bispecific antibody against tumors with HER2-low expression. Specifically, MC38, a mouse colon cancer cell, was genetically engineered to express human HER2 (MC38/hHER2; Biocytogen) and wild-type MC38 tumor cells were combined, based on the cell number, in a 3:7 ratio (MC38/hHER2 1.5×10⁶ cells+MC38 3.5×10⁶ cells, with 0.1 mL PBS) or a 1:9 ratio (MC38/hHER2 0.5×10⁶ cells+MC38 4.5×10⁶ cells, with 0.1 mL PBS) to prepare tumor cells that mimic the environment of low human HER2 expression. The above prepared tumor cells were implanted into the flanks of mice genetically engineered to express human 4-1BB (h4-1BB) (h4-1BB Knock In mice; Biocytogen), and mice were randomized based on tumor volume on day 4 after implantation.

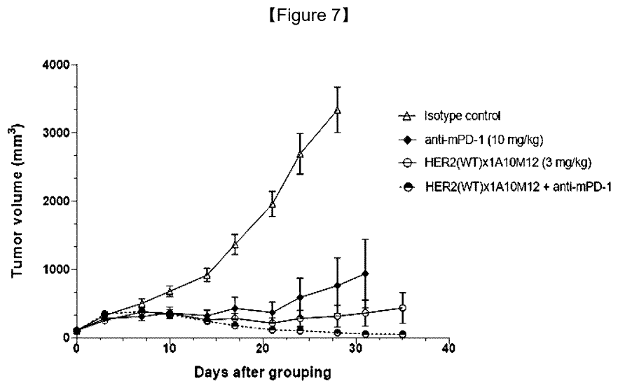

Mice that were grouped to have similar tumor volume were intraperitoneally administered with anti-hIgG1 antibody, US '250 Ab, and anti-HER2/anti-4-1BB bispecific antibody (HER2 (WT)×1A10 M12) at doses of 2.25 mg/kg, 3 mg/kg, and 3 mg/kg, respectively, on the day of grouping, 4 days later, and 7 days later, and then tumor volume was measured with a digital caliper twice a week. As shown in FIG. 6, the anti-HER2/anti-4-1BB bispecific antibody (HER2 (WT)×1A10 M12), compared to another anti-HER2/anti-4-1BB bispecific antibody, US '250 Ab and an anti-hIgG1 antibody, exhibited excellent tumor inhibition against both tumor mouse models (a 3:7 ratio model and a 1:9 ratio model) mimicking HER2-low expressing tumors.

### Example 9. Anti-Tumor Effects of Co-Administration of an Anti-HER2/Anti-4-1BB Bispecific Antibody and a PD-1 Inhibitor in a Mouse Model Bearing Tumors with HER2-Low Expression

A mouse model was constructed to evaluate the anti-tumor effects of co-administration of an anti-HER2/anti-4-1BB bispecific antibody and a PD-1 inhibitor (anti-PD-1 antibody) on tumors with HER2-low expression. Specifically, mouse colon cancer cells, MC38, genetically engineered to express human HER2 (MC38/hHER2; Biocytogen) and wild-type MC38 tumor cells were mixed in a 1:9 ratio (MC38/hHER2 0.5×10⁶ cells+MC38 4.5×10⁶ cells, with 0.1 mL PBS), by cell count, to prepare tumor cells that mimic an environment with low human HER2 expression. The above-prepared tumor cells were implanted into the flanks of mice genetically engineered to express human 4-1BB (h4-1BB) (h4-1BB knock-in mice; Biocytogen), and mice were randomized based on tumor volume on day 6 after implantation.

Mice that were grouped to have similar tumor volume, were intraperitoneally administered with,
anti-hIgG1 (2.25 mg/kg, BIW 2 weeks)+anti-mIgG2 antibody (10 mg/kg, QW 4 weeks) (Isotype control group),
anti-mPD-1 antibody (BioXcell, Cat #BP0146; 10 mg/kg, QW 4 weeks),
anti-HER2/anti-4-1BB bispecific antibody (HER2 (WT)×1A10 M12; 3 mg/kg, BIW 2 weeks), and
anti-HER2/anti-4-1BB bispecific antibody (HER2 (WT)×1A10 M12; 3 mg/kg, BIW 2 weeks)+anti-mPD-1 antibody (10 mg/kg, QW 4 weeks)
respectively in each group, and tumor volume was measured twice a week with a digital caliper.

As shown in FIG. 7, the anti-HER2/anti-4-1BB bispecific antibody (HER2 (WT)×1A10 M12) not only exhibited excellent anti-tumor effects when administered alone, but when co-administered with an anti-mPD-1 antibody, exhibited enhanced anti-tumor effects compared to each drug alone, against tumors mimicking a low HER 2 expressing environment.

All references, including publications, patent applications, and patents, cited in this specification are hereby incorporated by reference to the same extent as if each reference were individually and specifically incorporated by reference and set forth in its entirety herein.

The use of the terms “a” and “an” and “the” and “at least one” and “more than one” and similar references in the context of describing the present invention (particularly in the context of the following claims) shall be construed to include both the singular and the plural, unless otherwise specified herein or clearly contradicted by the context. The use of the term “at least one” (or “one or more”) followed by a list of one or more items (e.g., “at least one of A and B”) shall be construed to mean one selected item. Unless otherwise indicated herein or clearly contradicted by the context, the terms listed items (A or B) or any combination of two or more of the listed items (A and B), “comprising,” “having,” “including,” and “containing” shall be construed as open-ended terms (i.e., meaning “including but not limited to”) unless otherwise indicated. References in this specification to a range of values are intended merely to serve as a shorthand way of referring to each individual value falling within the range separately, unless otherwise indicated in this specification, and each individual value is incorporated into the specification as if it were individually recited herein. All methods described herein may be performed in any suitable order, unless otherwise indicated herein or clearly contradicted by the context. Any use of examples or exemplary language (e.g., “such as”) provided herein is merely to better illustrate the invention and does not limit the scope of the invention unless otherwise claimed. No language in the specification should be construed to indicate elements not claimed as essential to the practice of the invention.

Preferred embodiments of the invention are described herein, including the best mode known to the present inventors for carrying out the invention. Variations of these preferred embodiments may become apparent to those skilled in the art when reading the foregoing description. The inventors expect skilled artisans to use such modifications as appropriate, and the inventors intend the invention to be practiced differently than as specifically described herein. Accordingly, the invention includes all modifications and equivalents of the subject matter recited in the claims appended hereto as permitted by applicable law. Further, unless otherwise indicated herein or clearly contradicted by the context, any combination of the foregoing elements in all possible variations is included in the invention.

1. A method of treating or preventing an HER2 low-expressing cancer comprising administering a pharmaceutically effective amount of an anti-4-1BB/anti-HER2 bispecific antibody to a subject in need of treating or preventing the HER2 low-expressing cancer, wherein the anti-4-1BB/anti-HER2 bispecific antibody comprises:
(a) an anti-4-1BB antibody or an antigen-binding fragment thereof; and
(b) an anti-HER2 antibody or an antigen-binding fragment thereof,
and wherein the anti-4-1BB antibody or antigen-binding fragment thereof comprises:
an H-CDR1 comprising the amino acid sequence of SEQ ID NO: 1, 2, or 3;
an H-CDR2 comprising the amino acid sequence of SEQ ID NO: 4, 5, or 6;
an H-CDR3 comprising the amino acid sequence of SEQ ID NO: 7, 8, 9, 10, or 11;
an L-CDR1 comprising the amino acid sequence of SEQ ID NO: 12 or 13;
an L-CDR2 comprising the amino acid sequence of SEQ ID NO: 14 or 15; and
an L-CDR3 comprising the amino acid sequence of SEQ ID NO: 16 or 17.

2. The method according to claim 1, wherein the anti-4-1BB antibody or antigen-binding fragment thereof comprises:
an H-CDR1 comprising the amino acid sequence of SEQ ID NO: 1; an H-CDR2 comprising the amino acid sequence of SEQ ID NO: 4; an H-CDR3 comprising the amino acid sequence of SEQ ID NO: 8; an L-CDR1 comprising the amino acid sequence of SEQ ID NO: 12; an L-CDR2 comprising the amino acid sequence of SEQ ID NO: 14; and an L-CDR3 comprising the amino acid sequence of SEQ ID NO: 16.

3. The method according to claim 1, wherein the anti-4-1BB antibody or antigen-binding fragment thereof comprises:
a heavy chain variable region comprising the amino acid sequence of SEQ ID NO: 18, 19, 20, 21, 22, 23, 24, 25, 26, 27, 28, or 29; and
a light chain variable region comprising the amino acid sequence of SEQ ID NO: 30, 31, 32, 33, 34, or 88.

4. The method according to claim 1, wherein the anti-4-1BB antibody or antigen-binding fragment thereof of comprises:
(i) a heavy chain variable region comprising the amino acid sequence of SEQ ID NO: 25 and a light chain variable region comprising the amino acid sequence of SEQ ID NO: 34; or
(ii) a heavy chain variable region comprising the amino acid sequence of SEQ ID NO: 19 and a light chain variable region comprising the amino acid sequence of SEQ ID NO: 31.

5. The method according to claim 1, wherein the anti-4-1BB antibody or antigen-binding fragment thereof of comprises:
a heavy chain comprising the amino acid sequence of SEQ ID NO: 56, 57, 58, 59, 60, or 61; and
a light chain comprising the amino acid sequence of SEQ ID NO: 62, 63, or 64.

6. The method according to claim 1, wherein the anti-4-1BB antibody or antigen-binding fragment thereof is an anti-4-1BB scFv of the anti-4-1BB antibody.

7. The method according to claim 1, wherein the anti-HER2 antibody is trastuzumab, pertuzumab, or trastuzumab emtansine (T-DM1).

8. The method according to claim 1, wherein the anti-HER2 antibody or antigen-binding fragment thereof comprises:
an H-CDR1 comprising the amino acid sequence of SEQ ID NO: 65;
an H-CDR2 comprising the amino acid sequence of SEQ ID NO: 66;
an H-CDR3 comprising the amino acid sequence of SEQ ID NO: 67;
an L-CDR1 comprising the amino acid sequence of SEQ ID NO: 68;
an L-CDR2 comprising the amino acid sequence of SEQ ID NO: 69; and
an L-CDR3 comprising the amino acid sequence of SEQ ID NO: 70.

9. The method according to claim 1, wherein the anti-HER2 antibody or antigen-binding fragment thereof comprises:
a heavy chain variable region comprising the amino acid sequence of SEQ ID NO: 71; and
a light chain variable region comprising the amino acid sequence of SEQ ID NO: 72.

10. The method according to claim 1, wherein the anti-HER2 antibody or antigen-binding fragment thereof comprises:
a heavy chain comprising the amino acid sequence of SEQ ID NO: 73 or 74; and
a light chain comprising the amino acid sequence of SEQ ID NO: 75.

11. The method according to claim 1, wherein the anti-HER2 antibody or antigen-binding fragment thereof is an anti-HER2 scFv of the anti-HER2 antibody.

12. The method according to claim 1, wherein the anti-4-1BB/anti-HER2 bispecific antibody comprises:
(a) a full-length form of the anti-HER2 antibody, and
a scFv of the anti-4-1BB antibody; or
(b) (b) a full-length form of the anti-4-1BB antibody, and
a scFv of the anti-HER2 antibody.

13. (canceled)

14. The method according to claim 1, wherein the anti-4-1BB/anti-HER2 bispecific antibody comprises:
a first polypeptide comprising the amino acid sequence of SEQ ID NO: 83; and
a second polypeptide comprising the amino acid sequence of SEQ ID NO: 75.

15. The method according to claim 1, further comprising administering a cancer immunotherapy agent to the subject.

16. The method according to claim 15, wherein the cancer immunotherapy agent is a PD-1 inhibitor, a PD-L1 inhibitor, or a combination thereof.

17. The method according to claim 1, wherein the cancer has an immunohistochemistry (IHC) score of 0, 1+, or 2+ as measured by IHC using an anti-HER2 antibody specific for HER2.

18. The method according to claim 1, wherein the cancer has 50% or less of tumor cells expressing HER2 based on the total tumor cells.

19. The method according to claim 1, wherein the cancer has:
(a) a lower level of HER2 in the tumor than in a HCC1954 cell line as measured by a conventional protein measurement method; or
(b) an intratumoral HER2 level of 60% or less, 50% or less, 40% or less, 30% or less, or 20% or less relative to the HER2 level in a SK-BR-3 cell line, as measured by a conventional protein measurement method.

20. The method according to claim 1, wherein the cancer is characterized by FcγRI expression.

21. A method of treating or preventing an FcγRI-expressing cancer comprising administering a pharmaceutically effective amount of an anti-4-1BB/anti-HER2 bispecific antibody to a subject in need of treating or preventing an FcγRI-expressing cancer, wherein the anti-4-1BB/anti-HER2 bispecific antibody comprises:
(a) an anti-4-1BB antibody or an antigen-binding fragment thereof; and
(b) an anti-HER2 antibody or an antigen-binding fragment thereof,
and wherein the anti-4-1BB antibody or antigen-binding fragment thereof comprises:
an H-CDR1 comprising the amino acid sequence of SEQ ID NO: 1, 2, or 3;
an H-CDR2 comprising the amino acid sequence of SEQ ID NO: 4, 5, or 6;
an H-CDR3 comprising the amino acid sequence of SEQ ID NO: 7, 8, 9, 10, or 11;
an L-CDR1 comprising the amino acid sequence of SEQ ID NO: 12 or 13;
an L-CDR2 comprising the amino acid sequence of SEQ ID NO: 14 or 15; and
an L-CDR3 comprising the amino acid sequence of SEQ ID NO: 16 or 17.

22-34. (canceled)
